(19) United States
(12) Patent Application Publication
(10) Pub. No.: US 2011/0306071 A1
(43) Pub. Date: Dec. 15, 2011

(54) Compositions and Methods for Identifying Substrate Specificity of Inhibitors of Gamma Secretase

(75) Inventors: I. Paul Shapiro, Half Moon Bay, CA (US); Guriqbal S. Basi, Palo Alto, CA (US); Zhao Ren, San Mateo, CA (US)

(73) Assignee: ELAN PHARMACEUTICALS, INC., South San Francisco, CA (US)

(21) Appl. No.: 13/043,278

(22) Filed: Mar. 8, 2011

Related U.S. Application Data
(62) Division of application No. 12/172,978, filed on Jul. 14, 2008, now abandoned.
(60) Provisional application No. 60/949,738, filed on Jul. 13, 2007.

(30) Foreign Application Priority Data
Jul. 14, 2008 (US) PCT/US08/70012

(51) Int. Cl. G01N 33/577; C12Q 1/37; G01N 33/53

(52) U.S. Cl. 435/7.94; 435/7.92; 435/23

(57) ABSTRACT

The invention provides assays and methods for determining the substrate specificity of gamma secretase inhibitors and for identifying substrate-selective (and substrate isoform-selective) inhibitors of gamma secretase. The invention provides assays and methods for determining whether a compound inhibits gamma secretase in a site specific or substrate specific manner. The invention provides isolated polypeptide sequences comprising modified gamma secretase substrates, and polynucleotide sequences encoding the polypeptide sequences. The invention also provides compounds that inhibit gamma secretase, pharmaceutical compositions comprising such compounds, and methods of treating Alzheimer's disease using such compounds.

This application is a divisional application of U.S. patent application Ser. No. 12/172,978, filed Jul. 14, 2008, which claims the benefit of priority to U.S. Provisional Patent Application Ser. No. 60/949,738, filed Jul. 13, 2007, each of which is incorporated by reference herein in its entirety.

### FIELD OF THE INVENTION

The invention is related to the treatment of Alzheimer's disease. More particularly, the invention relates to assays, reagents and methods for identifying compounds that preferentially inhibit gamma (γ)-secretase cleavage of APP-like substrates relative to other substrates for gamma secretase.

### BACKGROUND OF THE INVENTION

Accumulation of brain β-amyloid is the major pathological feature of Alzheimer's disease. The generation of A-beta (Aβ) from amyloid precursor protein (APP) is a complex process requiring successive cleavages by two proteases, beta-(β-) and gamma-(γ-) secretase (Selkoe, D. J., Physiol. Rev. (2001) 81:741-766). β-secretase is a membrane-bound aspartyl protease that cleaves APP on its luminal portion (Sinha, S., et al., Nature (1999) 402:537-540; Vassar, R., et al., Science (1999) 286:735-741; Yan, R., et al., Nature (1999) 402:533-537; Lin, X., et al., Proc Natl Acad Sci USA., (2000) 97:1456-1460), producing a carboxyl-terminal (C-terminal) fragment consisting of 99 amino acids (C99/β-CTF). The β-CTF/C99 can be subsequently cleaved by gamma secretase at two major sites within the transmembrane domain (TMD), γ and ε, generating Aβ and an intracellular fragment known as APP intracellular domain (AICD) (Sastre, M., et al., EMBO Rep. (2001) 2:835-841; Weidemann, A., et al., Biochemistry (2002) 41:2825-2835). These γ and ε cleavages occur near the middle and near the cytoplasmic face of the transmembrane domain (TMD), respectively. Some experimental evidence shows that gamma secretase cleavage of gamma secretase substrates, in particular of APP and Notch, occurs sequentially with the cleavage at epsilon preceding cleavage at gamma. Furthermore, it has been established that epsilon-site cleavage is independent of gamma cleavage, while gamma-site cleavage occurs after and depends on prior epsilon cleavage (Zhao, G., et al., J. Biol. Chem., (2004); 279:50647-50; Qi-Takahara, Y., et al., J. Neurosci., (2005); 25:436-45). Alternatively, an α-secretase-dependent processing of APP results in a shorter α-CTF/C83 fragment that can undergo similar cleavages (Selkoe, D. J., Physiol. Rev. (2001) 81:741-766). Gamma secretase is also known to cleave Notch, CD44 and numerous other type I transmembrane proteins (De Strooper B., Neuron (2003) 38:9-12). The amino acid sequence requirement for gamma secretase-dependent cleavage around the cleavage site(s) within the transmembrane domain seems relatively relaxed, depending more on the size of the extracellular domain of a substrate than the recognition of specific sequences (Struhl, G., and Adachi, A., Molecular Cell (2000) 6:625-636). The Notch processing resembles that of APP, with two homologous gamma secretase cleavage sites S4 and S3 positioned in the middle of the TMD and near the cytoplasmic leaflet, respectively (Hartmann, D., et al., J. Mol. Neurosci. (2001) 17:171-181; Okochi, M., et al., EMBO J. (2002) 21:5408-5416). Notchβ and Notch intracellular domain (NICD) are the two cleavage products, with the latter being an important transcriptional activator (Mumm, J. S., and Kopan, R., Dev. Biol. (2000) 228:151-165). Four distinct Notch transmembrane receptor isoforms (Notch1-4), two Notch transmembrane ligands (Delta and Jagged) and gamma secretase are among the key elements in Notch signaling and related processes. Many other substrates for gamma secretase are known to possess two or more intra-membrane cleavage sites (i.e., in the TMD) analogous to the γ and ε cleavage sites of APP, and the S4 and S3 cleavage sites of Notch.

Gamma secretase is a multi-subunit aspartyl protease that consists of at least four different membrane proteins, presenilin (PS), Nicastrin, Aph-1 and Pen-2 (De Strooper B., Neuron (2003) 38:9-12). PS is thought to be the catalytic subunit of the holoenzyme, containing two conserved intramembrane aspartate residues essential for substrate cleavage (Wolfe, M. S., et al., Nature (1999) 398:513-517; Kimberly, W. T., et al., J. Biol. Chem. (2000) 275:3173-3178). The precise mechanisms by which gamma secretase recognizes and cleaves its substrates remain elusive, partly because these proteolytic events occur within a hydrophobic environment of membrane lipid bilayer.

The same (or highly similar) gamma secretase enzyme activity appears to be involved in processing APP, Notch and other substrates. Gamma secretase cleaves numerous type-I, single membrane spanning protein substrates within their transmembrane domain, a process sometimes referred to as Regulated Intramembrane Proteolysis (RIP). Many gamma-secretase substrates participate in diverse physiologic and disease processes. In many instances, nuclear signaling activity of these substrates depends on gamma secretase processing, followed by nuclear translocation and subsequent gene activation by the liberated intracellular domains (ICDs). Inhibition of Notch processing (at the S3/epsilon site) is a major undesirable effect of non-selective gamma secretase inhibitors. Thus, the identification and development of gamma secretase inhibitors with selectivity for inhibiting gamma secretase activity at any particular gamma secretase substrate, such as APP relative to Notch is an important objective for successful development of effective and well tolerated gamma secretase inhibitors.

One possible way to reduce gamma secretase activity for any given gamma secretase substrate, such as reducing Aβ production without significantly affecting other gamma secretase substrates, is to identify inhibitors of gamma secretase that preferentially inhibit gamma secretase activity at the gamma cleavage site relative to the epsilon cleavage site of the other substrates (e.g., APP and Notch). Another possible way to reduce gamma secretase activity for any given gamma secretase substrate, such as reducing Aβ production without significantly affecting other gamma secretase substrates, is to identify inhibitors of gamma secretase that are specific inhibitors for the substrate (e.g., specific for APP over Notch). The identification of such inhibitors would provide additional therapeutic candidates for use in treating a wide range of conditions that are related to gamma secretase processing of a substrate molecule, such as cancer or AD, and those inhibitors would exhibit fewer deleterious side effects. Thus, there is a need in the art to provide a simple method for screening compounds to identify such inhibitors.

The inventors herein provide compositions and methods for identifying compounds that inhibit gamma secretase in a substrate specific manner, as well as methods for identifying compounds that inhibit cleavage preferentially at the gamma cleavage site of APP compared to cleavage at the epsilon cleavage site of APP and compared to cleavage of other gamma secretase substrates.

### SUMMARY OF THE INVENTION

In one aspect, the invention is directed to a method for determining whether a compound inhibits gamma secretase in a substrate specific matter. The method includes:
(a) contacting a first gamma secretase substrate comprising a gamma cleavage site with the compound and gamma secretase under conditions that allow for gamma secretase activity;
(b) separately contacting a second gamma secretase substrate comprising a gamma cleavage site with the compound and gamma secretase under conditions that allow for gamma secretase activity;
(c) determining the amount of gamma secretase activity at the gamma cleavage site of the first substrate and the second substrate;
(d) comparing the amounts of gamma secretase activity at the gamma cleavage site from step (a) with the amount of gamma secretase activity at the gamma cleavage site from step (b) and determining that the compound inhibits gamma secretase in a substrate specific manner when the amount of gamma secretase activity at the gamma cleavage site from step (a) is different from step (b).

In various aspects of the invention, the first gamma secretase substrate is a naturally occurring substrate such as, for example, amyloid precursor protein (APP), Notch, amyloid precursor-like protein (APLP2), tyrosinase, CD44, erbB4, n-cadherin, p75 NTFR, and SCNB2.

The method of the invention also includes a first gamma secretase substrate that is a first polypeptide having a first juxtamembrane domain sequence [JMD1] and a transmembrane domain sequence [TMD1], and a second gamma secretase substrate that is a second polypeptide having a second juxtamembrane domain sequence [JMD2] and the transmembrane domain sequence [TMD1] of the first gamma secretase substrate. For example, the [TMD1] is the transmembrane domain of APP and [JMD1] and [JMD2] are juxtamembrane domains independently selected from APLP2, Notch, erbB4, tyrosinase, p75 NTFR, SCNB2, n-cadherin, and CD44, wherein [JMD1] and [JMD2] are not the same.

The method of the invention further includes a second gamma secretase substrate that includes the formula:

[JMDΔC4]-X1-X2-X3-X4-[TMD] (Formula II);
wherein,
JMDΔC4 comprises the amino acid sequence of a juxtamembrane domain (JMD) sequence of a gamma secretase substrate, wherein the JMD lacks the four C-terminal peptides;
[TMD] comprises a transmembrane domain sequence of a gamma secretase substrate; and
X1, X2, X3, and X4 are independently selected from any amino acid.

In a particular embodiment X1 is selected from S, T, G, P, Q, R, V, L, N, P, A, K, E, I, F, H, W, and D; X2 is any amino acid; X3 is selected from S, N, D, P, E, R, T, F, I, K, L, V, G, W, H, and A; and X4 is any amino acid.

In a particular embodiment X1 is selected from S, T, G, P, Q, R, V, L, N, P, A, K, E, I, F, H, W, and D; X3 is selected from S, N, D, P, E, R, T, F, I, K, L, V, G, W, H, and A; and X2 and X4 are selected from L, I, H, E, V, A, S, T, D, N, P, K, Q, and R.

In a particular embodiment X1 is selected from S, T, G, P, Q, R, and D; X2 is any amino acid; X3 is selected from S, N, D, P, and A; and X4 is any amino acid.

In a particular embodiment, the (JMD) has the juxtamembrane domain of a gamma secretase substrate of one of APLP2, Notch, erbB4, tyrosinase, p75 NTFR, SCNB2, n-cadherin, and CD44 and the TMD has the transmembrane domain of one of APLP2, Notch, erbB4, tyrosinase, p75 NTFR, SCNB2, n-cadherin, and CD44.

A further aspect of the invention includes a method for determining whether a compound selectively inhibits gamma secretase activity at a first gamma secretase substrate relative to a second gamma secretase substrate. The method comprises
(a) contacting a first transfected cell culture with the compound at various concentrations under conditions that allow for gamma secretase activity;
(b) contacting a second transfected cell culture with the compound at various concentrations under conditions that allow for gamma secretase activity;
(c) measuring ICD produced by each of the first and second transfected cell cultures at each of the various compound concentrations to generate a first dose response curve of the effect of the compound on the first transfected cell culture and a second dose response curve of the effect of the compound on the second transfected cell culture; and
(d) comparing the first and second dose response curves.

In this aspect, the first transfected cell culture is transfected with a first polynucleotide encoding a first polypeptide having a juxtamembrane domain sequence (JMD1) and a transmembrane domain sequence (TMD1) of the formula [JMD1][TMD1], wherein [JMD1] is from a first gamma secretase substrate; and a second transfected cell culture is transfected with a second polynucleotide encoding a second polypeptide having a juxtamembrane domain sequence (JMD2) and a transmembrane domain sequence (TMD1) of the formula [JMD2][TMD1], wherein [JMD2] is from a second gamma secretase substrate and the TMD1 of the first and second polypeptides is the same.

Also, in this aspect, a shift in the second dose response curve toward a higher concentration relative to the first dose response curve indicates that the compound is selective for the first gamma secretase substrate relative to the second gamma secretase substrate.

In various embodiments of this aspect, the first gamma secretase substrate is APP, APLP2, Notch, erbB4, tyrosinase, p75 NTFR, SCNB2, n-cadherin, or CD44. Therefore, [TMD1] of the formulas [JMD1][TMD1] and [JMD2][TMD1] is the transmembrane domain of APP and [JMD1] and [JMD2] are juxtamembrane domains independently selected from APLP2, Notch, erbB4, tyrosinase, p75 NTFR, SCNB2, n-cadherin, and CD44, wherein [JMD1] and [JMD2] are not the same.

In various embodiments of the cell culture assays of the invention, active gamma secretase is endogenously and constitutively produced by the cell cultures. Cell cultures can include, for example, HEK293 cells. ICD can be measured using a monoclonal antibody that specifically binds to VMLKKKC (SEQ ID NO:39).

In yet another aspect, the invention includes a method for determining whether a compound selectively inhibits gamma secretase activity of a first gamma secretase substrate relative to a second gamma secretase substrate. The method includes:
(a) contacting a first transfected cell culture with the compound at various concentrations under conditions that allow for gamma secretase activity;
(b) contacting a second transfected cell culture with the compound at various concentrations under conditions that allow for gamma secretase activity;
(c) measuring ICD produced by each of the first and second transfected cell cultures at each of the various compound concentrations to generate a first dose response curve of the effect of the compound on the first transfected cell culture and a second dose response curve of the effect of the compound on the second transfected cell culture; and
(d) comparing the first and second dose response curves.

In this method, the first transfected cell culture can be transfected with a polynucleotide encoding a first polypeptide comprising Formula II:

[JMDΔC4]-X1-X2-X3-X4-[TMD]
wherein
[JMDΔC4] comprises the amino acid sequence of a juxtamembrane domain (JMD) sequence of a gamma secretase substrate, wherein the JMD lacks the four C-terminal peptides;
[TMD] comprises a transmembrane domain sequence of a gamma secretase substrate; and
X1-X2-X3-X4 are independently selected from any amino acid; and the second transfected cell culture is transfected with a second polynucleotide encoding a second polypeptide comprising Formula II:

[JMDΔC4]-X1-X2-X3-X4-[TMD]
wherein [TMD] and [JMDΔC4] are as defined above, and
   X1-X2-X3-X4 are independently selected from any amino acid.

In a particular embodiment X1 is selected from S, T, G, P, Q, R, V, L, N, P, A, K, E, I, F, H, W, and D; X2 is any amino acid; X3 is selected from S, N, D, P, E, R, T, F, I, K, L, V, G, W, H, and A; and X4 is any amino acid.

In a particular embodiment X1 is selected from S, T, G, P, Q, R, V, L, N, P, A, K, E, I, F, H, W, and D; X3 is selected from S, N, D, P, E, R, T, F, 1, K, L, V, G, W, H, and A; and X2 and X4 are selected from L, I, H, E, V, A, S, T, D, N, P, K, Q, and R.

In a particular embodiment X1 is selected from S, T, G, P, Q, R, and D; X2 is any amino acid; X3 is selected from S, N, D, P, and A; and X4 is any amino acid.

In one embodiment of this aspect, X1-X2-X3-X4 of the first polypeptide is from a first gamma secretase substrate, and X1-X2-X3-X4 of the second polypeptide is from a second gamma secretase substrate.

In one aspect of this method, a shift in the second dose response curve toward a higher concentration relative to the first dose response curve indicates that the compound is selective for the first gamma secretase substrate relative to the second gamma secretase substrate.

In particular embodiments, X1-X2-X3-X4 of the first and second polypeptide are independently selected from GLNK, SLSS, GSNK, GSNS, PPAQ, SSNK, GSSK, QHAR, QASR, TTDN, RDST, DVDR, or QIPE. The [TMD] of the first and second polypeptide can include SEQ ID NO:13. [JMDΔC4] of the first and second polypeptide can be independently selected from SEQ ID NOs: 3-5, and 7-12.

In particular examples, the polypeptide of Formula II includes one of the following sequences:

(e)(C99GVP-APLP2):
(SEQ ID NO: 16)
LEDAEFRHDS GLEEERESVG PLREDFSLSS GAIIGLMVGG
VVIATVIVIT LVML;
(f)(C99GVP-NOTCH1):
(SEQ ID NO: 17)
LEDAEFRHDS GPYKIEAVQS ETVEPPPPAQ GAIIGLMVGG
VVIATVIVIT LVML;
(g)(C99GVP-SREBP1):
(SEQ ID NO: 18)
LEDAEFRHDS GAKPEQRPSL HSRGMLDRSR GAIIGLMVGG
VVIATVIVIT LVML;
(h)(C99APPD4-APLP2):
(SEQ ID NO: 42)
LEDAEFRHDS GYEVHHQKLV FFAEDVSLSS GAIIGLMVGG
VVIATVIVIT LVML;
(i)(C99-APP-(G25S):
(SEQ ID NO: 43)
LEDAEFRHDS GYEVHHQKLV FFAEDVSSNK GAIIGLMVGG
VVIATVIVIT LVML;
(j)C99-APP-(S26L):
(SEQ ID NO: 44)
LEDAEFRHDS GYEVHHQKLV FFAEDVGLNK GAIIGLMVGG
VVIATVIVIT LVML;
(k)C99-APP-(N27S):
(SEQ ID NO: 45)
LEDAEFRHDS GYEVHHQKLV FFAEDVGSSK GAIIGLMVGG
VVIATVIVIT LVML;
(l)C99-APP-(K28S):
(SEQ ID NO: 46)
LEDAEFRHDS GYEVHHQKLV FFAEDVGSNS GAIIGLMVGG
VVIATVIVIT LVML;
(m)(C99APPA4-NOTCH1):
(SEQ ID NO: 100)
LEDAEFRHDS GYEVHHQKLV FFAEDVPPAQ GAIIGLMVGG
VVIATVIVIT LVML;
(n)(C99APPM-SREBP1):
(SEQ ID NO: 101)
LEDAEFRHDS GYEVHHQKLV FFAEDVDRSR GAIIGLMVGG
VVIATVIVIT LVML;
(o)(C99GVP-APLP2-gsnk):
(SEQ ID NO: 19)
LEDAEFRHDS GLEEERESVG PLREDFGSNK GAIIGLMVGG
VVIATVIVIT LVML;
(p)(C99GVP-NOTCHI-gsnk):
(SEQ ID NO: 20)
LEDAEFRHDS GPYKIEAVQS ETVEPPGSNK GAIIGLMVGG
VVIATVIVIT LVML;
and
(q)(C99GVP-SREBP1-gsnk):
(SEQ ID NO: 21)
LEDAEFRHDS GAKPEQRPSL HSRGMLGSNK GAIIGLMVGG
VVIATVIVIT LVML.

In particular aspects X2 is serine and X4 is lysine, or X2 is leucine and X4 is serine.

### BRIEF DESCRIPTION OF THE DRAWINGS

FIG. 1: Schematic diagram of gamma and epsilon cleavage on APP and NotchΔE. (A) Generalized location of the α-secretase, β-secretase, and γ-secretase cleavage sites in APP C99 and the corresponding γ-secretase sites in NotchΔE, S4 and S3. FIG. 1A also illustrates three alternative potential mechanistic models for APP/Aβ>Notch/NICD selectivity of gamma secretase inhibitors; cleavage site; length of N-terminal end of substrate; primary sequence of the substrate. (B) Sequences of major (thicker arrows) and minor (narrower arrows) gamma-secretase γ and ε cleavage sites in APP C99 and the corresponding S4 and S3 γ-secretase sites in NotchΔE.

FIG. 2: Effect of point mutations on Aβ40 generated by gamma secretase. Several point mutations (S26L and K28S) within the APP juxtamembrane domain at the GSNK residues adjacent to the transmembrane domain have an effect on the amount of Abeta40 generated by gamma secretase. The upper panel shows the amount of Aβ40 generated by gamma secretase in: cell only control (HEK); wildtype APP (APP-WT); the S26L mutation (APP-S26L); and the K28S mutation (APP-K28S). The lower panel shows that the expression of each mutant gamma substrate is normalized to the wildtype expression level.

FIG. 3: Schematic diagram for assay incorporating Aβ and AICD ELISAs and AICD luciferase activation. In the upper panel, the schematic shows the general arrangement of the α-, β-, and γ-secretase (γ and ε) cleavage sites in APP and the location of the JMD; the middle schematic shows the general arrangement of the C99-GVP amino acid sequence incorporating the GVP insert and shows the Aβ and AICD (with GVP insert) fragments generated by γ-secretase cleavage and their detection by Aβ and AICD ELISAs. The lower panel shows how the AICD fragment comprising the GVP transactivation domain can bind and activate the luciferase reporter gene system.

FIG. 4: Description of several non-limiting chimera sequences of various JMD swap domains and point mutations. These sequences can further comprise an N-terminal LEDAEFRHDSGk-sequence (SEQ ID NO:37) and a C-terminal—VHHQKLVFFA EDVGSNKGAI IGLMVGGVVI ATVIVITLVM LKKK*QYTSIH HGVVEVDAAV TPEERHLSKM QQNGYENPTY KFFEQMQN sequence (SEQ ID NO:38), with the C-terminal end of the GVP sequence attached to or inserted at the any point in SEQ ID NO:38. Certain non-limiting construct insert the GVP sequence at the “*” noted in SEQ ID NO:38.

FIG. 5: Cleavage profile of chimeric substrate molecules. (A) Schematic view of γ-secretase dependent processing of C99GVP and the luciferase reporter assay measuring AICD production. Two proteolytic sites (γ and ε) within the TMD region are shown (solid arrows) as is the cleavage site of the (LE) signal peptide (open arrow) (B) Western blot using antibodies 2H3, anti-VP16, anti-APP, or anti-AICD neo-epitope antibody such as 22B11 demonstrating that AICD-GVP (solid arrowheads) is generated from C99GVP in a γ-secretase dependent manner. Open arrowheads identify C83GVP, an N-terminal truncation of C99GVP. HEK transfected cells treated without inhibitor (lanes 1, 2), with (24 hr) the γ-secretase inhibitor L-685,458 (lanes 3 (1 μM), 4 (20 μM)), or with DAPT (lanes 5 (1 μM), 6 (20 μM)). (C) Gamma secretase inhibitors block luciferase reporter transactivation by AICD-GVP (generated from C99-GVP) in a dose-dependent matter. L-685,458 and DAPT in HEK293 cells transfected with luciferase reporter. Data are mean (+/−SD) luminescence units of three independent experiments. *, p<0.01 versus reporter only (control); **, p<0.05 versus C99GVP-transfected cells with no inhibitor. (D) Inhibitors block secreted Aβ. Media from cells treated (24 h) with DMSO, L-685,458 (5 μM) or DAPT (5 μM) and analyzed for Aβ40 (hatched) and Aβ42 (open) using sandwich ELISA. The capture/detection antibody pairs were 2G3/2H3 and 21F12/2H3. Data are mean+/−SD of three experiments. Total secreted Aβ (bottom panel) in the same media was immunoprecipitated and analyzed via Western blot using the 2H3 antibody; *, p<0.01 versus Aβ40 from C99GVP transfectants treated with DMSO, **, p<0.01 versus Aβ42 from same group. (E) Inhibitors show equal Aβ potency with C99GVP and native substrate. Cells transfected with either APP or C99GVP treated with serially diluted L-685,458 (left panel) or DAPT (right panel) and analyzed by ELISA (expressed as percentage of DMSO-treated controls). Calculated IC₅₀ values are included in the inset tables in each panel. (F) Subcellular distribution of C99GVP and AICD-GVP in transfected COS-7 cells: (N) nuclear staining present in DMSO-treated cells is abolished after addition of DAPT (5 μM), allowing for clearer representation of C99GVP expression (bottom right panel).

FIG. 6: C99-GVP is a functional gamma secretase substrate undergoing physiological cleavages and effects of JMD chimeras. (A) Schematic view and amino acid sequences C99GVP and several JMD chimeras, indicating the γ and ε cleavage sites in the TMD and the epitopes recognized by antibodies used in Aβ-immunobased detection and analysis (SEQ ID NOs: 16, 17, 18). (B) Immunoblots using 2H3 (top panel) and anti-APP (middle panel) antibodies of cell lysates from transiently transfected HEK cells treated with DMSO or DAPT (5 μM). The bottom panel shows immunoblot of conditioned media was collected from the same samples, prior to cell lysis. (C) JMD chimeras and reporter activity. Luciferase assay of cells treated with DMSO (grey bars) or DAPT (5 μM, black bars) at 48 h post-transfection. Data is presented as a percentage of DMSO-treated C99GVP control. (D) and (E) JMD chimeras and effect on secreted Aβ40 (D) and Aβ42 (E). ELISA analysis of conditioned media from luciferase assays is with data expressed as a percentage of DMSO-treated C99GVP control. (F) JMD chimeras do not inhibit interaction with gamma-secretase. Immunoblots of cell lysates from transfected HEK cells using antibody against an N-terminal fragment of PS-1 or APP demonstrate that C99GVP and JMD chimeras bind similarly to PS-1. Solid arrowhead indicates AICD-GVP fragment. (G) Subcellular distribution of JMD chimeras in COS-7 cells showing nuclear staining (N) which is abolished by DAPT treatment. The DAPT treated cells a homogeneous expression profile of the JMD chimeras.

FIG. 7: C99-GVP juxtamembrane domain swaps differentially affect secreted Aβ and AICD production. (A) The effect of an α-secretase inhibitor on secreted Aβ40. Conditioned media from cells treated with DMSO (grey bar) or 40 μM TAPI-1 (black bars) were collected and analyzed by ELISA specific for Aβ40. Data expressed as percentage of the TAPI-1 treated C99GVP control. *, p<0.01; **, p<0.05. (B) The effect of Aβ-degrading enzyme inhibitors on secreted Aβ40. Conditioned media from cells treated with DMSO (gray) or 40 μM phosphoramidon plus 1 mg/mL Bacitracin (checked bars) analyzed by ELISA specific for Aβ40. Data expressed as percentage of the inhibitor-treated C99GVP control; *, p<0.01. (C) Shows intracellular accumulation of longer Aβ species in HEK cells transfected with C99GVP or C99GVP-APLP2: synthetic Aβ peptide standards (lane 1), Cell lysate (lane 2, 4), conditioned media (lane 3, 5), Aβ′ peptide standard derived from C99GVP-APLP2 (lane 6).

FIG. 8: The GSNK motif in the APP JMD plays a role in gamma secretase cleavage. (A) Expression profile of modified JMD chimeric substrates retaining the GSNK motif from APP. The sequence alignments (SEQ ID NOs: 15, 19, 20, and 21) highlight the differences between the sequences in the JMD region (top panel). Middle panel shows a 2H3 antibody immunoblot of cell lysates from transfected HEK cells treated with DMSO or DAPT (5 μM). Bottom panel shows a APP antibody immunoblot of same cell lysates. Open arrowheads indicate C83GVP-like fragments derived from substrates. (B) JMD chimeras show luciferase reporter transactivation mediated by the AICD-GVP fragment in cells after treated with DMSO (grey bars) or DAPT (5 μM, black bars) at 48 hr. post-transfection. Data is expressed as percentage of activity compared to DMSO treated C99GVP control. (C) The JMD chimeras exhibit normal Aβ secretion. Western blot of conditioned media from DMSO-treated cells using the 2H3 antibody (bottom panel) was quantified by densitometry using a synthetic Aβ40 peptide standard, expressed as percentage of C99GVP control. (D) The JMD chimeras exhibit normal Aβ40 secretion. Aβ40 ELISA analysis of conditioned media collected from DMSO (grey bars) or DAPT (black bars) treated cells. Data is expressed as percentage of DMSO-treated C99GVP control.

FIG. 9: Mapping juxtamembrane residues involved with efficient gamma cleavage. (A). Expression profile of the new mutant substrates that contain point mutations in the GSNK motif with alignment of C99GVP sequences with point mutants, with the substituted residues indicated by underline (SEQ ID NOs: 15, 42, 43, 44, 45, and 46. Immunoblot using 2H3 antibody (middle panel) or anti-APP antibody (bottom panel) of cell lysates from transfected HEK cells treated with DMSO or DAPT (5 μM) (bottom panel). Open arrowhead indicates C83GVP-like fragment derived from substrate(s) (B) Immunoprecipitation and Western blot (2H3 antibody) of conditioned media from cells treated with DMSO (bottom panel). The top panel shows quantification by densitometry, expressed as a percentage of the C99GVP control. (C) Aβ40 ELISA analysis of conditioned media from DMSO-treated (grey bars) or DAPT-treated cells, expressed as percentage of DMSO-treated C99GVP control. (D) Luciferase signal (48 h. post-transfection) of cells treated with DMSO (grey bars) or DAPT (5 μM, black bars) indicate that mutations do not induce change in AICD-GVP mediated reporter transactivation. Data expressed as percentage of DMSO-treated C99GVP control.

FIG. 10: Standard curve from AICD sandwich ELISA is shown using the synthetic AICD standard, which includes AICD₁₋₆ plus Cys (SEQ ID NO:39), a spacer, and AICD₃₆₋₄₈ (SEQ ID NO:40). The AICD₅₀ native standard sequence is also shown (SEQ ID NO:41).

FIG. 11: Concurrent inhibition by non-selective inhibitors of Aβ and AICD. (A) Aβ ELISA; (B) AICD ELISA; (C) Immunoblot using anti-APP C-terminal antibody (Sigma) which reveals inhibition of AICD-DD and stabilization of chimeric CTFs with increasing concentrations of gamma-inhibitors. (D) APP γ versus ε selectivity of non-selective, published compounds (Elan's 44989 and 46719) relative to other gamma secretase inhibitors, DAPT and L-685,458 (Merck).

FIG. 12: Concurrent inhibition by ELAN sulfonamides (APP/Aβ>Notch/NICD selective gamma secretase inhibitors) of Aβ and AICD; (A) A-beta ELISA; (B) AICD ELISA. These compounds form a class of gamma secretase inhibitors that can have selectivity for APP over other gamma substrates, such as Notch. The ELISA results demonstrate that the inhibitors act on the gamma and epsilon sites in APP.

FIG. 13: Inhibition of AICD generation from chimeric C99-GVP with selective and non-selective inhibitors. (A) AICD ELISAs for selective and non-selective inhibitors with wildtype C99 (APP) and the chimeric JMD swaps, C99-APLP2 and C99-Notch; (B) Summary of data in (A) demonstrating the selectivity of inhibitor compounds 475516 and 477899 for the native APP substrate compared to the APP-chimeric JMD substrates (APLP2: 42.2 and 26.2; Notch; 33.6 and 15.9, respectively).

FIG. 14: Relative potencies of selective and non-selective compounds for inhibition of AICD production from chimeric JMC C99GVP substrates. (A) IC₅₀s (average IC₅₀s from two replicate concentration-response experiments) for AICD inhibition with selective compound 475516 for the various C99-GVP constructs (APP (SEQ ID NO:47); APLP2 (SEQ ID NO:48); Notch (SEQ ID NO:49); Notch-GNSK (SEQ ID NO:50); SLSS (SEQ ID NO:51)) were normalized to the IC₅₀ for C99-GVP with WT APP JMD (error bars indicate CVs based on replicate determinations of IC₅₀). (B) IC₅₀ values for AICD inhibition with non-selective compound 44989 (single determination) for the various constructs were normalized to the IC₅₀ for C99-GVP with WT APP JMD. (C) IC₅₀ values (single IC₅₀ from pooled data from the two replicate concentration-response experiments) for AICD inhibition with compound 475516 for the various constructs were normalized to the IC₅₀ for C99-GVP with WT APP JMD. (D) Inhibition of gamma secretase production of AICD using selective sulfonamide inhibitors and C99 chimeric substrate sequences: wild type (C99GVP-APP); C99GVP-Notch; C99GVP-APPΔ4-SLSS; C99GVP-NotchΔ4-GSNK. Retention of the native APP JMD region, or the GSNK sequence located adjacent to the N-terminal end of the transmembrane region reduces the ratio of gamma secretase AICD produced by gamma secretase in the presence of selective inhibitor compounds 480271 and 48970.

FIG. 15. Specificity of MAb 22B11 for the AICD neo-epitope is demonstrated by the ability of excess AICD neo-epitope peptide to compete in a concentration-dependent manner for binding in an ELISA assay experiment, while a peptide and a protein spanning the cleavage site both fail to compete. These data also suggest the K_{d} of 22B11 for binding to AICD neoepitope is ˜5 nM.

FIG. 16. The AICD ELISA detects AICD in cell lysates. A sandwich ELISA using AICD neo-epitope monoclonal 22B11 for capture detects increasing amounts of gamma-secretase-generated AICD-DD in extracts from HEK293 cells expressing increasing amounts of APP substrate (from increasing concentrations transiently transfecting of Fas-APP-DD cDNA).

FIG. 17. The baseline, uninhibited levels of various gamma secretase cleavage products in cell lysates from HEK293 cells transfected with JMD constructs derived from various different substrates and in the absence of gamma secretase inhibitor treatment. The constructs include C99-Notch, C99-ErbB4; C99-APLP2; C99-p75NTFR; C99-N-Cadherin; C99-SCNB2; C99-tyrosinase; and control untransfected cells. The data is presented normalized to the amount of products (ICD, Abeta40, Abeta42, and C99) for the C99-APP-GVP construct cleavage products. Thus, all cleavage products for the C99-APP construct are expressed as 100%, and the products from the other substrates tested are shown relative to the respective products from C99-APP construct.

FIG. 18. Relative potency of selective vs. non-selective inhibitors for inhibition of ICD from various JMD constructs. The constructs include C99-APP; C99-Notch, C99-ErbB4; C99-APLP2; C99-p75NTFR; C99-SCNB2; and C99-tyrosinase. The data is presented normalized to the EC₅₀ value for inhibition of AICD production from C99-APP, and thus represents “x-fold” relative selectivity of the compounds for the various substrates.

FIG. 19. Effect on the potency of non-selective di-benzocaprolactam (ELN-44989) and selective sulfonamide (ELN-475516 and ELN-481090) gamma secretase inhibitors as a function of amino acid mutagenesis at the GSNK amino acid sequence of the APP JMD region. The corresponding amino acids from the JMD of APLP2 were inserted as series of point mutants as well as a full four amino acid substitution in C99-APP-GVP.

### DETAILED DESCRIPTION OF THE INVENTION

The invention provides a convenient and simple system for monitoring cleavage mediated by gamma secretase on known or postulated substrates of gamma secretase using a single modular construct. The various aspects of the invention provide a portable system for monitoring the effects of substrate identity and structural variations on inhibitor potencies for gamma secretase cleavage. Relative potencies between substrates in the system can be used to deduce inhibitor selectivity among the different substrates and substrate variants.

The assays described herein are modular, single-format assay systems which measure substrate selectivity of gamma-secretase inhibitory compound(s). Since the assays enable the determination of gamma inhibitor potencies against, and consequently selectivity between, multiple gamma secretase substrates, they can be used to discover gamma secretase inhibitors with any desired profile of substrate selectivity. For example, this assay can be used to discover compounds useful for treating Alzheimer's disease by identifying APP-selective compounds that inhibit Abeta production while not modulating physiologic processing of Notch.

Similarly, research has shown that diverse human cancers, including T-cell acute lymphoblastic leukemias (T-ALL), carcinomas of the breast, prostate and pancreas and CNS neoplasms may involve aberrantly high isoform specific Notch signaling, suggesting that Notch-isoform specific gamma secretase inhibitors may have therapeutic benefit. Evidence also suggests that Notch is involved in diseases including autoimmune, proliferative and inflammatory diseases of a wide range of end organs (Arumugam Thiruma, et al., Nat. Med., (2006) 12(6): 621-3; Barsky Sanford, H., et al., FASEB J. (2007); Jurynczyk, M., et al. (2005) J. Neuroimmunol., 170(1-2): 3-10; Kogoshi, H., et al., Oncology Reports (2007)18(1): 77-80; Liu, H., et al., Br. Cancer Res. and Treatment (2006); Meng Raymond, D., et al. (2006) Proceedings of the American Association for Cancer Research Annual Meeting; Nefedova, Y., et al., Blood (2008) 111(4): 2220-9; Setoeuchi, T., et al., J. Bone and Min. Res. (2007); Sun, Y, et al., Br. Cancer Res. and Treatment (2006); Teachey David, T., et al., Blood (2008)111(2): 705-14; van Es Johan, H. and H. Clevers, Trends Molec. Med. (2005) 11(11): 496-502; Zhang, P., et al. (2006) Proceedings of the American Association for Cancer Research Annual Meeting). The emerging understanding of Notch dependent cancers and autoimmune indications suggest specific isoforms of Notch are critical to the respective disease in question, e.g., T-cell leukemias (Vacca, et al., EMBO J. (2006) 25(5): 1000-8; Bellavia, D., et al., EMBO J, (2007) 26(6): 1670-80) (Ellisen, L. W., et al., Cell (1991) 66: 649-661; Nickoloff, B. J., et al., Oncogene (2003) 22: 6539-6608) and EAE (Jurynczyk, M. A., et al., J Immunol (2008) 180(4): 2634-40). Thus, for these Notch-dependent cancers (and other conditions, such as autoimmune disorders), the assays and methods described herein can be used to identify gamma-secretase inhibitors that are selective for a particular Notch isoform, and that spare normal processing of the other Notch isoforms which are not associated with disease. Thus, the assays and methods described herein can be used to identify compounds that demonstrate isoform selectivity for a particular gamma secretase substrate that is involved with any disease indication, including but not limited to Alzheimer's disease, cancer and autoimmune indications.

One of skill in the art will recognize that the methods and assays described herein can be used advantageously to identify compounds with a favorable inhibitory profile for any currently known or subsequently identified gamma secretase substrate.

In part, the invention addresses one of the primary challenges in discovering gamma secretase inhibitors for treatment of AD. For instance, in addition to APP processing (resulting in Aβ production), gamma secretase is now recognized to process many other substrates. One notable other substrate is Notch. Clinical development of gamma secretase inhibitors is limited by the fact that these compounds inhibit processing of Notch at potencies equal to the inhibition of Aβ production from APP. Inhibition of Notch processing by these non-selective gamma-secretase inhibitors is known to result in mechanistic toxicity (primarily in the GI tract) in pre-clinical safety models (e.g., rat and dog). In addition, gamma secretase has been demonstrated to process an ever expanding list of known substrates, any one of which could also manifest as mechanistic toxicity if its cleavage by gamma secretase is inhibited at potencies equal to that of APP.

Prior to this invention, assessing the selectivity of any gamma secretase modulator for modulating APP cleavage versus any one of the other known gamma secretase substrates involved a labor intensive series of steps requiring expression of each substrate under study, plus development and use of separate and distinct assays for quantifying cleavage of that substrate by gamma secretase, each conducted under different conditions and requiring detection of a different cleavage product. The invention provides solutions to existing problems, including a) assessing selectivity of gamma secretase modulators using a single assay format with highly similar substrates and a common read-out (instead of running and comparing measurement of cleavage products from two different types of assays), and b) easily identifying other substrate(s) of gamma secretase, in addition to APP, and whose processing may be modulated by apparently APP selective compounds.

The invention provides methods used to identify compounds that preferentially modulate γ-secretase activity on a particular γ-secretase substrate relative to another γ-secretase substrate. Some methods involve screening compounds in an assay that uses gamma secretase substrate having the transmembrane domain (TMD) of, for example, APP along with the juxtamembrane domain (JMD) of a different gamma secretase substrate or a JMD having modifications to its amino acid sequence. The substrate can further include various other polypeptide sequences for stability of the substrate in its intracellular domain and to provide a moiety that can be used to detect the various cleavage products that result from cleavage of the substrate by gamma secretase. Therefore, a universal substrate having a variable JMD is provided wherein the JMD of the substrate is derived from various gamma secretase substrates. Using a single substrate with a variable JMD, the potency of gamma secretase modulators can be determined and related to potency of the modulator on natural substrates from which the JMD is copied or derived. Accordingly, the invention provides a method of investigating the selectivity of a gamma secretase modulator on various gamma secretase substrates without the need of testing the inhibitor on the natural substrate.

The invention also provides methods used to identify compounds that preferentially modulate gamma secretase activity on a particular gamma secretase substrate at either the gamma (γ) or epsilon (ε) cleavage sites of the substrate relative to other gamma secretase substrates. The assays can employ known methods of detecting gamma secretase cleavage products. In addition, the invention provides a monoclonal antibody for the detecting of cleavage products (e.g., ICD). The invention also provides methods for identifying a gamma secretase substrate for which certain classes of gamma secretase inhibitors have an increased or decreased inhibitory potency, relative to another gamma secretase substrate. Some methods can be used for identifying a compound that preferentially modulates γ-secretase cleavage of APP substrate at the γ-cleavage site relative to cleavage of Notch substrate the S3/ε-cleavage site.

Before describing the present invention in further detail, a number of terms will be defined. As used herein, the singular forms “a,” “an,” and “the” include plural referents unless the context clearly dictates otherwise.

The terms “gamma secretase substrate,” “γ-secretase substrate,” and “substrate for gamma secretase” are all used interchangeably herein, and refer to a protein or polypeptide that is processed (i.e., cleaved/proteolyzed) by the multi-subunit protease, gamma secretase, under conditions that allow for gamma secretase activity. Some non-limiting examples of a gamma secretase substrate include those described herein, such as amyloid precursor protein (APP), Notch, amyloid precursor-like protein (APLP2), tyrosinase, CD44, erbB4, n-cadherin and SCNB2, and the like. Gamma secretase substrates also include any isotypes (isoforms) of known gamma secretase substrates such as, for example, Notch1, Notch2, Notch3, and Notch4. Further, gamma secretase substrates are not limited to human sequences, but also include substrates from other mammals (orthologs), including mouse, rat, guinea pig, primates and the like. The term “substrate molecule” refers to a synthetic, chimeric and/or recombinant polypeptide that can be processed (i.e., cleaved/proteolyzed) by the multi-subunit protease, gamma secretase, under conditions that allow for gamma secretase activity. The term “naturally occurring gamma secretase substrate” or “native substrate” refers to a non-chimeric polypeptide derived from amyloid precursor protein (APP), Notch, amyloid precursor-like protein (APLP2), tyrosinase, CD44, erbB4, n-cadherin or SCNB2, or other non-chimeric, naturally occurring polypeptides that serve as a gamma secretase substrate, including isoforms thereof. One example of a naturally occurring gamma secretase substrate is a polypeptide comprising the JMD and TMD from APP. Some gamma secretase substrates and substrate molecules, including naturally occurring gamma secretase substrates, can be expressed in a cell endogenously or recombinantly as transmembrane proteins or polypeptides.

As used herein, “conditions that allow for gamma secretase activity” refers to conditions, either in vitro or in vivo (e.g., cell-based assays) that comprise gamma-secretase enzyme under conditions that allow the expression of cDNAs encoding the substrate molecules of the invention, and allowing normal expression, maturation and trafficking of the exogenously expressed substrate molecules, and for normal gamma secretase activity. Such conditions include those that allow for proliferation of cells in culture, including typical cell culture conditions, as gamma secretase activity is usually present in cells in which it is expressed. Certain non-limiting examples of such conditions are provided herein in the Examples section, and include cell culture in high glucose DMEM supplemented with 10% fetal bovine serum and 50 units/ml penicillin and streptomycin, at 37° C. and 5% CO₂. Other specific conditions that allow for cell growth as well as in vitro buffer systems are known to those of skill in the art. Those of skill in the art also understand that gamma secretase is robust and active under a number of conditions and in a variety of cell types, and can be expressed using a number of expression systems/vectors. Thus, a variety of expression vector/host systems may be utilized to contain and express the polynucleotide molecules encoding the chimeric gamma secretase substrates of the invention. These systems include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Mammalian cells that are useful in recombinant protein productions include but are not limited to VERO cells, HeLa cells, Chinese hamster ovary (CHO) cell lines, COS cells (such as COS-7), W138, BHK, HepG2, 3T3, RIN, MDCK, A549, PC12, K562 and 293 cells.

Accordingly, the methods and amino acid sequences of the invention can be performed and produced using any expression system and in any cell type that allows for gamma secretase activity or that allows for expression of the amino acid sequences. Those of skill will be able to identify such types of cells, such as the non-limiting examples disclosed herein, including HEK-293 cells and COS cells.

As used herein the term “beta peptide” or “β-peptide” means the N-terminal product from cleavage of a gamma secretase substrate at the gamma cleavage site. For example, Aβ and Notch1-β are beta peptides that result from gamma secretase cleavage of the substrates APP and Notch1, respectively.

By “intracellular domain,” “intracellular domain peptide,” “intracellular domain fragment,” or “ICD” is meant the C-terminal product derived from cleavage of a gamma secretase substrate at the gamma (γ) or epsilon (ε) site. Typically, ICD results from cleavage at the most cytoplasmically-proximal site (such as the ε site), but may be at γ site for some substrates that lack two gamma secretase cleavage sites within their transmembrane domain (TMD). For example, AICD and NICD are intracellular domain peptides that result from gamma secretase cleavage of APP and Notch1 at their ε/S3 cleavage site, respectively.

The terms “gamma” and “epsilon” are generally used herein with respect to a particular cleavage site on a gamma secretase substrate. These terms are taken to mean the two distinct cleavage sites within the TMD at which gamma secretase acts on a substrate, where cleavage at the gamma site generates the C-terminus of β peptide (e.g., Aβ₄₀ or Aβ₄₂); and cleavage at the epsilon site generates the N-terminus of intracellular domain peptides, (e.g., AICD, NICD, etc.). Cleavage at the gamma site in the absence of epsilon cleavage will also generate ICD and Aβ-like peptides.

By “C99GVP” is meant the polypeptide sequence of the 99 amino acid C-terminal fragment of APP resulting from cleavage of APP by β-secretase, into which a Gal4-VP16 DNA-binding/transactivation domain is inserted in-frame, three amino acid residues C-terminal to the end of the transmembrane domain. Examples of this type of polypeptide include such non-limiting sequences as those described in Karlstrom, H., et al., J. Biol. Chem., (Mar. 1, 2002); 277(9):6763-6766.

By “transmembrane domain,” “transmembrane region,” or “TMD” is meant the region of a gamma secretase substrate that is located within the lipid bilayer of the cellular membrane. In general, the TMD is hydrophobic and is bounded at the N and C termini by charged residues. As used herein, the transmembrane domain of the several gamma secretase substrates (e.g., APP and Notch) contains both sites at which gamma secretase cleaves the substrate, i.e., the gamma and S3/epsilon cleavage sites. The N-terminus of the TMD abuts the C-terminus of the juxtamembrane domain of the substrate. For example, the C-terminus of the JMD of APP is located at about residue 28 of SEQ ID NO:1, and the N-terminus of the TMD of APP is located at about residue 29 of SEQ ID NO:1. The region within a type I integral membrane protein (where “type I” is characterized by the C-terminus being located in the cytosolic/lumenal side of the membrane) containing the transmembrane domain (TMD) is a section of polypeptide, typically hydrophobic and not containing any charged residues, often alpha helical, which passes through or “spans” a membrane. TMDs average about 20 amino acid residues in length and can be predicted computationally using methods known to those of skill in the art, including hydropathy analysis algorithms and a variety of other experimental techniques including but not limited to x-ray diffraction. TMDs are often bounded, or “bookended,” on either or both faces by hydrophilic and charged residues. In certain aspects of the invention, the JMD of the substrate extends N-terminally to the extracellular side of the TMD (N-terminal side of the TMD) for a length of 15-20 residues, commonly about 19 residues. The TMD can comprise amino acid sequence that binds specifically to a specific binding agent, such as a polyclonal or monoclonal antibody.

“Juxtamembrane domain” or “JMD” as used herein refers to the region of a gamma secretase substrate that is located immediately to the N-terminal side of the transmembrane region. The juxtamembrane domain is typically about 15 to about 30 amino acids in length, and usually about 19 to about 25 amino acids in length. As used herein, JMDΔC4 refers to a JMD lacking the four C-terminal peptides located immediately adjacent to the N-terminal end of the transmembrane domain (TMD).

The terms “AGBP¹” and “AGBP²” as used herein are meant include an amino acid sequence having an epitope or covalently attached moiety that is part of a specific binding pair. Examples of such sequences include either internal or neo-epitopes with the native Abeta sequence recognized by antibodies, epitopes within the last 10-15 residues of APP C-terminus recognized by antibodies, AICD neo-epitope (generated by gamma-secretase cleavage at the epsilon site) and epitope tags on either the N- or C-terminal ends of the substrate including but not limited to HA-tag, myc-tag, and the like, that are recognized by antibodies.

The term “Sig” is used herein to designate a general amino acid signal sequence that functions to direct transport and/or translocation of a polypeptide to which it is attached to a particular cellular or extracellular location. Such signal sequences are well known in the art (see, e.g., Devillers-Thiery A, et al., “Homology in amino-terminal sequence of precursors to pancreatic secretory proteins” Proc Natl Acad Sci USA. 1975 December; 72(12):5016-5020).

In one aspect, the invention provides methods and assays for determining whether a compound inhibits gamma secretase in a substrate specific matter. The method includes contacting a two or more gamma secretase substrates that have gamma cleavage site with gamma secretase and one or more compounds that modulate and gamma secretase activity under conditions that allow for gamma secretase activity. The contacting step can include in vivo conditions such as cell-based assays, or can be conducted in vitro. After an appropriate amount of time, the amount of gamma secretase activity at the gamma cleavage site for each substrate is determined. The activities can be compared to determine whether the compound(s) inhibit activity in a substrate specific manner. For example, when the amount of activity with one substrate is different than the activity with a second substrate, it can be determined that the compounds inhibit gamma secretase in a substrate specific manner.

In an embodiment of this aspect of the invention, one or more gamma secretase substrates can be a naturally-occurring substrates such as, for example, a gamma secretase substrate selected from amyloid precursor protein (APP), Notch, amyloid precursor-like protein (APLP2), tyrosinase, CD44, erbB4, n-cadherin, p75 NTFR, and SCNB2. For example, a first gamma secretase substrate is APP and a second gamma secretase substrate is APLP2, Notch, erbB4, tyrosinase, p75 NTFR, SCNB2, n-cadherin, or CD44.

In another embodiment, the one or more gamma secretase substrates have the same transmembrane domains [TMD], but different juxtaposition membranes domains [JMD]. For example, a first gamma secretase substrate is a first polypeptide including the formula [JMD1][TMD1], wherein [JMD1] comprises a first juxtamembrane domain sequence and [TMD1] includes a transmembrane domain sequence, and the second gamma secretase substrate is a second polypeptide including the formula [JMD2][TMD1], wherein [JMD2] includes a second juxtamembrane domain sequence and [TMD1] is as defined above, wherein the juxtamembrane domain sequences and transmembrane domain sequence are as described herein, including the juxtamembrane domain and transmembrane regions of any of the other currently known gamma secretase substrates (see, e.g., Beel and Sanders Cell. Mol. Life. Sci. (2008) 65:1311-1334). In one embodiment [TMD1] is the transmembrane domain of APP and [JMD1] and [JMD2] are juxtamembrane domains independently selected from APLP2, Notch, erbB4, tyrosinase, p75 NTFR, SCNB2, n-cadherin, and CD44, as well as potential/putative gamma secretase substrates, wherein [JMD1] and [JMD2] are not the same sequence. In other aspects of the invention additional substrates having constant TMDs but differing JMDs can be used to compare the substrate selectivity of gamma secretase modulating compounds.

In a further embodiment of this aspect of the invention, the second gamma secretase substrate includes a peptide of Formula II:

[JMDΔC4]-X1-X2-X3-X4-[TMD]
wherein,
JMDΔC4 comprises the amino acid sequence of a juxtamembrane domain (JMD) sequence of a gamma secretase substrate, wherein the JMD lacks the four C-terminal peptides;
[TMD] comprises a transmembrane domain sequence of a gamma secretase substrate; and
X1, X2, X3, and X4 are independently selected from any amino acid.

In a particular embodiment X1 is selected from S, T, G, P, Q, R, V, L, N, P, A, K, E, I, F, H, W, and D; X2 is any amino acid; X3 is selected from S, N, D, P, E, R, T, F, I, K, L, V, G, W, H, and A; and X4 is any amino acid.

In a particular embodiment X1 is selected from S, T, G, P, Q, R, V, L, N, P, A, K, E, I, F, H, W, and D; X3 is selected from S, N, D, P, E, R, T, F, I, K, L, V, G, W, H, and A; and X2 and X4 are selected from L, I, H, E, V, A, S, T, D, N, P, K, Q, and R.

In a particular embodiment X1 is selected from T, G, P, Q, R, and D; X2 is any amino acid; X3 is selected from S, N, D, P, and A; and X4 is any amino acid.

In another embodiment, the methods or assays of the invention include contacting two or more transfected cell cultures with one or more compounds having gamma secretase modulating activity at various concentrations under conditions that allow for gamma secretase activity, and then measuring the amount of ICD produced from gamma secretase cleavage in the transfected cell cultures at each of the various compound concentrations. Each of the cell cultures is transfected with a polynucleotide encoding gamma secretase substrate. Dose response curves of the effect of the compounds on each of the transfected cell cultures are determined and compared. For example, a first transfected cell culture is transfected with a polynucleotide encoding a first polypeptide, and the second transfected cell culture is transfected with a second polynucleotide encoding a second polypeptide, each polypeptide comprising Formula II:

[JMDΔC4]-X1-X2-X3-X4-[TMD]

wherein [JMDΔC4], [TMD], and X1-X2-X3-X4 are as defined herein, and wherein Formula II does not define the same sequence for both the first and second polypeptides. In this method, a shift in the second dose response curve toward a higher concentration relative to the first dose response curve indicates that the compound is selective for the first gamma secretase substrate relative to the second gamma secretase substrate (or vice versa).

The methods and assays of this aspect of the invention have a wide range of utility, which will be appreciated by one of skill in the art. Using any combination or permutation of gamma secretase inhibitor compounds (or candidate inhibitor compounds) and gamma secretase substrates, the selectivity profile of any compound, or selectivities for any series of gamma secretase inhibitor compounds, for any gamma secretase substrate can be determined against one or more gamma secretase substrates. Similarly, the methods and assays can be used to identify an inhibitor compound from a series (a plurality) of inhibitor compounds that has the best selectivity between two (or more) gamma secretase substrates. For example, when the method or an assay includes two different gamma secretase substrates (SBT1 and SBT2), and those substrates are contacted with a series of inhibitor compounds (e.g., ten inhibitor compounds, CMP1, CMP2, CMP3, etc.) at several concentrations, a series of dose response curves for each of the inhibitor compounds can be generated and analyzed for each of the two (or more) substrates. The IC₅₀ value for each compound against each substrate can be determined and expressed as a ratio of IC₅₀ values (i.e., [IC₅₀ of CMP1 for SBT1]: [IC₅₀ of CMP1 for SBT2]). This “selectivity ratio” can be used to determine which of the inhibitor compounds has the best (or worst) selectivity and to rank order the compounds with respect to selectivity for the substrates used in the assay.

In one aspect the invention provides a substrate molecule for gamma secretase. The substrate molecule can comprise a chimeric polypeptide sequence including the TMD from one species of gamma secretase substrates, e.g., APP, and the JMD from a second substrate, e.g. Notch. In some substrate molecules, the C-terminus of the JMD is attached to the N-terminus of the TMD. The gamma secretase activity on the cleavage of the gamma and/or epsilon cleavage sites within the TMD of the substrate can be modulated by exchanging the JMD of the substrate. One such substrate molecule can be represented by formula I:

JMD(1)-TMD(2) (Formula I)

wherein JMD(1) is the JMD of a first gamma secretase substrate, and TMD(2) is the TMD of a second gamma secretase substrate.

Some chimeric polypeptides include the TMD from a gamma secretase substrate, e.g., APP, and the JMD from the same substrate or a second substrate, e.g. Notch, where one or more of the four C-terminal amino acids of the native JMD sequence have been modified. It has been found that the modification of the four C-terminal amino acids can modulate the activity of gamma secretase on the cleavage of the gamma or epsilon sites in the TMD of the chimeric substrate.

One such chimeric polypeptide can be represented by Formula II:

[JMDΔC4]-X1-X2-X3-X4-[TMD] (Formula II);

wherein,
JMDΔC4 comprises the amino acid sequence of a juxtamembrane domain (JMD) sequence of a gamma secretase substrate, wherein the JMD lacks the four C-terminal peptides;
[TMD] comprises a transmembrane domain sequence of a gamma secretase substrate; and
X1, X2, X3, and X4 are s independently elected from any amino acid;
   with the provisos that
when JMD of [JMDΔC4] is the JMD of APP, and [TMD] comprises the transmembrane domain sequence of APP, X1-X2-X3-X4 is not G-S-N-K;
when JMD of [JMDΔC4] is the JMD of APLP2; and [TMD] comprises the transmembrane domain sequence of APLP2, X1-X2-X3-X4 is not S-L-S-S;
when JMD of [JMDΔC4] is the JMD of Notch 1, and [TMD] comprises the transmembrane domain sequence of Notch1, X1-X2-X3-X4 is not P-P-A-Q;
when JMD of [JMDΔC4] is the JMD of erbB4, and [TMD] comprises the transmembrane domain sequence of erbB4, X1-X2-X3-X4 is not Q-H-A-R;
when JMD of [JMDΔC4] is the JMD of tyrosinase, and [TMD] comprises the transmembrane domain sequence of tyrosinase, X1-X2-X3-X4 is not Q-A-S-R;
when JMD of [JMDΔC4] is the JMD of p75 NTFR, and [TMD] comprises the transmembrane domain sequence of p75 NTFR, X1-X2-X3-X4 is not T-T-D-N;
when JMD of [JMDΔC4] is the JMD of SCNB2, and [TMD] comprises the transmembrane domain sequence of SCNB2, X1-X2-X3-X4 is not R-D-S-T;
when JMD of [JMDΔC4] is the JMD of n-cadherin, and [TMD] comprises the transmembrane domain sequence of n-cadherin, X1-X2-X3-X4 is not D-V-D-R; and
when JMD of [JMDΔC4] is the JMD of CD44, and [TMD] comprises the transmembrane domain sequence of CD44, X1-X2-X3-X4 is not Q-I-P-E.

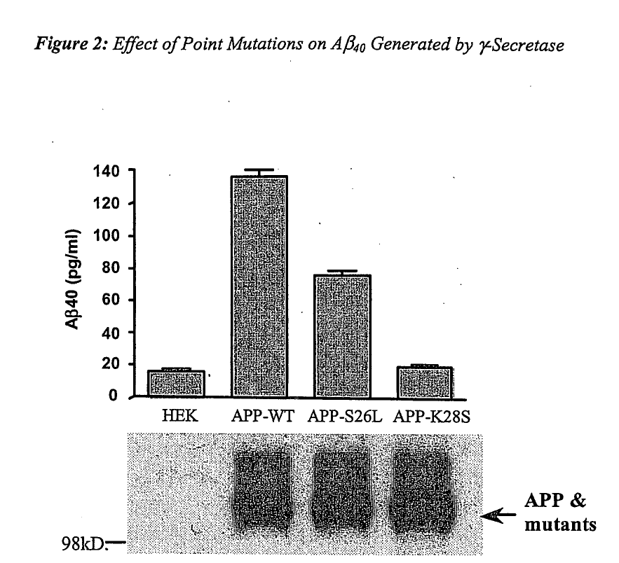

Certain of the four C-terminal amino acids (X1-X4) may play a greater role in determining the specificity or cleavage efficiency that gamma secretase has for a particular cleavage site or for a particular substrate sequence. Thus, using routine techniques known in the art, a series of mutagenesis experiments can be designed that can identify the optimal amino acid(s) for these particular sequences. For example, X2 and X4 may play a role in determining gamma secretase's substrate specificity (see, e.g., FIG. 2). Thus, a particular native JMD can be selected, and a series of amino acid mutants can be made wherein all the residues except those corresponding to X2 and X4 are kept consistent with the native sequence, while residues X2 and X4 are varied using the twenty naturally occurring amino acids. An assay measuring gamma secretase activity can be used to screen the resulting mutant sequences for those which exhibit the largest change in gamma secretase activity. In certain embodiments of the invention, X2 and X4 are selected from L, I, H, E, V, A, S, T, D, N, P, K, Q, and R.

Similarly, using mutagenesis experiments, a series of chimeric substrates can be generated that comprise optimized amino acid residues at X2 and X4, which are kept constant, while the remaining other residues are mutagenized using the twenty naturally occurring amino acids. Utilizing the same type of screening assay allows for identification of mutant chimeric substrates that are further optimized for selectivity for any given gamma secretase inhibitor, and/or for gamma secretase selectivity.

The polypeptides of Formulas I and II can comprise additional amino acid sequence(s) covalently linked to either the N-terminal or the C-terminal ends of the polypeptide, or both. One polypeptide comprises additional amino acid sequence attached to the C-terminal end of the TMD portion, wherein the additional amino acid sequence comprises at least a portion of an intracellular domain (ICD) sequence from a gamma secretase substrate. For example, the ICD sequence is selected from the ICD of APP (AICD), Notch1 (NICD), APLP2, tyrosinase, CD44, erbB4, SCNB2, n-cadherin, p75 NTFR, and the like.

In some polypeptides, the sequence at the C-terminus of the TMD includes an additional amino acid sequence that can be used to transactivate certain reporter genes, provide a sequence or moiety that can be recognized by a specific binding agent, and/or provide for increased stabilization of the ICD sequence. In one example, this additional amino acid sequence includes a GVP sequence (e.g. SEQ ID NO:2). The additional sequence can be inserted into, behind or in front of the ICD sequence, as long as the GVP sequence does not affect the immunogenicity, of the ICD when such property is required for the detection of the ICD (for example, binding of an antibody that recognizes ICD). As an alternative, the GVP sequence provides a means for detecting the ICD. For example, the GVP is a member of a reporter system that can be detected in a luciferase assay by measuring expression changes from Gal4-luciferase regulated expression plasmids.

The polypeptides of Formula I and II can include an additional amino acid sequence covalently attached to the N-terminal end of the JMD portion wherein the additional amino acid sequence is a sequence or moiety that can be recognized by a specific binding agent. The sequence N-terminal of the JMD(1) or JMDΔC4 can include a signal peptide sequence that can direct transport of the polypeptide to an intracellular or extracellular location and can direct the insertion of the gamma secretase substrate into and across a cellular membrane (where it can contact the gamma secretase). For example, the additional amino acid sequence covalently attached to the N-terminal end of JMD(1) or JMDΔC4 can include the N-terminal sequence of a gamma secretase substrate, selected from APP, Notch1, APLP2, tyrosinase, CD44, erbB4, p75 NTFR, n-cadherin, SCNB2, and the like. The signal sequence can be attached to the N-terminal sequence of the gamma secretase substrate through a linker, such as an amino acid sequence that directs site specific cleavage by a peptidase, proteinase, or other enzyme that cleaves peptide bonds (e.g., L (leu)-E (glu)-sequence).

Another polypeptide can be represented as Formula III and Formula IV:

[Sig]-LE-[AGBP¹]-JMD(1)-TMD(2)-[AGBP²] (Formula III)

[Sig]-LE-[AGBP¹]-[JMDΔC4]-X1-X2-X3-X4-[TMD][AGBP²] (Formula Iv)

In Formulas III and IV, JMD(1), TMD(2), JMDΔC4 and TMD are as described above for Formula I and II. In addition:
[Sig] is optional and includes a signal peptide that directs transport of the polypeptide for insertion of the substrate into and across the appropriate cellular membrane;
LE is the dipeptide Leu-Glu, and is optional;
[AGBP¹] includes antigenic amino acid sequence, preferably from a sequence of a beta-like peptide derived from APP, Notch1, APLP2, tyrosinase, CD44, erbB4, p75 NTFR, n-cadherin, and SCNB2;
[AGBP²] includes the intracellular domain (ICD) sequence of a gamma secretase substrate, wherein the ICD sequence comprises a second antigenic amino acid sequence having at least one specific binding determinant for a specific binding agent, and can optionally include a stabilizing sequence or reporter sequence such as GVP;
X1 is selected from S, G, P, Q, R, and D;
X2 is selected from L, S, P, T, V, D, A, I, and R;
X3 is selected from S, N, D, P, and A; and
X4 is selected from K, S, Q, N, T, E, and R.

In Formula IV, [JMDΔC4] is selected from YEVHHQKLVFFAEDV (APP, SEQ ID NO.3); LEEERESVGPLREDF (APLP2, SEQ ID NO.4); PYKIEAVQSETVEPP (NOTCH1, SEQ ID NO.5); HDCIYYPWTGHSTLP (erbB4, SEQ ID NO: 7; (NM_{—}001042599)); SDPDSFQDYIKSYLE (tyrosinase, SEQ ID NO: 8; (NM_{—}000372)); VTTVMGSSPVVTRG (p75 NTFR, SEQ ID NO: 9; (NM_{—}002507.1)); HGKIHLQVLMEEPPE (SCNB2, SEQ ID NO: 10; (NM_{—}004588)); LRVKVCQCDSNGDCT (n-cadherin, SEQ ID NO: 11 (NM_{—}001792)); and QEGGANTTSGPIRTP (CD44, SEQ ID NO: 12; (NM_{—}000610)).

Also, TMD(2) or [TMD] can comprise the transmembrane domain sequence of any gamma secretase substrate, such as the non-limiting example of the TMD of APP: GAIIGLMVGG VVIATVIVIT LVML (SEQ ID NO.13). In both Formula III and I, the JMD portion of the sequence is not identical to JMD of the natural substrate containing the TMD. Therefore, the provisos associated with Formula I apply to Formula III.

In some polypeptides, [AGBP¹] of Formulas III and IV includes an N-terminal sequence of a gamma secretase substrate, selected from APP, Notch1, APLP2, tyrosinase, CD44, erbB4, SCNB2, p75 NTFR, n-cadherin and the like. While the sequence including [AGBP¹] will often conveniently be a portion of, or derived from an N-terminal sequence of a gamma secretase substrate, [AGBP¹] can further provide a sequence that allows for detection and quantification by any known method, such as by specific binding assays (e.g., ELISA). Accordingly, in some polypeptides, [AGBP¹] comprises the sequence DAEFRHDSG (Abeta N-terminal epitope) (SEQ ID NO:14).

In some polypeptides, [AGBP²] of Formulas III and IV includes at least a portion of an intracellular domain (ICD) sequence from a gamma secretase substrate, selected from APP (AICD), Notch1 (NICD), APLP2, tyrosinase, CD44, erbB4, SCNB2, p75 NTFR, n-cadherin and the like, such as, for example, [AGBP²] comprises the amino acid sequence of SEQ ID NO:38 (AICD).

Several non-limiting examples of sequences of LE-[AGBP¹]-[JMDΔC4]-X1-X2-X3-X4-[TMD] of Formula IV include the following:

(a) (C99GVP-APLP2):
(SEQ ID NO: 16)
LEDAEFRHDS GLEEERESVG PLREDFSLSS GAIIGLMVGG
VVIATVIVIT LVML;
(b) (C99GVP-NOTCH1):
(SEQ ID NO: 17)
LEDAEFRHDS GPYKIEAVQS ETVEPPPPAQ GAIIGLMVGG
VVIATVIVIT LVML;
(c) (C99GVP-SREBP1):
(SEQ ID NO: 18)
LEDAEFRHDS GAKPEQRPSL HSRGMLDRSR GAIIGLMVGG
VVIATVIVIT LVML;
(d) (C99APPA4-APLP2):
(SEQ ID NO: 42)
LEDAEFRHDS GYEVHHQKLV FFAEDVSLSS GAIIGLMVGG
VVIATVIVIT LVML;
(e) (C99-APP-(G255):
(SEQ ID NO: 43)
LEDAEFRHDS GYEVHHQKLV FFAEDVSSNK GAIIGLMVGG
VVIATVIVIT LVML
(f) C99-APP-(S26L):
(SEQ ID NO: 44)
LEDAEFRHDS GYEVHHQKLV FFAEDVGLNK GAIIGLMVGG
VVIATVIVIT LVML
(g) C99-APP-(N27S):
(SEQ ID NO: 45)
LEDAEFRHDS GYEVHHQKLV FFAEDVGSSK GAIIGLMVGG
VVIATVIVIT LVML
(h) C99-APP-(K28S):
(SEQ ID NO: 46)
LEDAEFRHDS GYEVHHQKLV FFAEDVGSNS GAIIGLMVGG
VVIATVIVIT LVML
(i) (C99APPM-NOTCH1):
(SEQ ID NO: 100)
LEDAEFRHDS GYEVHHQKLV FFAEDVPPAQ GAIIGLMVGG
VVIATVIVIT LVML;
(j) (C99APPA4-SREBP1):
(SEQ ID NO: 101)
LEDAEFRHDS GYEVHHQKLV FFAEDVDRSR GAIIGLMVGG
VVIATVIVIT LVML;
(k) (C99GVP-APLP2-gsnk):
(SEQ ID NO: 19)
LEDAEFRHDS GLEEERESVG PLREDFGSNK GAIIGLMVGG
VVIATVIVIT LVML;
(l) (C99GVP-NOTCH1-gsnk):
(SEQ ID NO: 20)
LEDAEFRHDS GPYKIEAVQS ETVEPPGSNK GAIIGLMVGG
VVIATVIVIT LVML;
and
(m) (C99GVP-SREBP1-gsnk):
(SEQ ID NO: 21)
LEDAEFRHDS GAKPEQRPSL HSRGMLGSNK GAIIGLMVGG
VVIATVIVIT LVML.

In some polypeptides, the GVP includes the sequence KLLSSIEQAC DICRLKKLKC SKEKPKCAKC LKNNWECRYS PKTKRSPLTR AHLTEVESRL ERLEQLFLLI FPREDLDMIL KMDSLQDIKA LLTGLFVQDN VNKDAVTDRL ASVETDMPLT LRQHRISATS SSEESSNKGQ RQLTVSGIPG DLAPPTDVSL GDELHLDGED VAMAHADALD, DFDLDMLGDG DSPGPGFTPH DSAPYGALDM ADFEFEQMFT DALGIDEYGG (SEQ ID NO:2). In some polypeptides, the GVP sequence can be modified by any routine molecular biological technique, such as conservative amino acid substitutions, amino acid insertions and deletions, C- and/or N-terminal truncations, and the like, so long as it retains the desired function of the sequence, for example, transactivation of a signal sequence, providing a recognition or binding moiety, and/or increasing stability to the polypeptide fragment resulting from gamma secretase cleavage. Accordingly, the invention encompasses functional equivalents to the above GVP sequence, including sequences that are about 80% to about 100% identical to SEQ ID NO:2 (i.e., sequences having about 80, 85, 90, 95, 96, 97, 98, or 99% identity to SEQ ID NO:2).

The gamma secretase substrates of formulas I-IV can be used in assays that measure the activity of gamma secretase on the substrates. Some assays include the steps of (a) contacting a polypeptide sequence of Formulas I-IV with gamma secretase under conditions that allow for gamma secretase activity, for example, by contacting a cell with a test compound, wherein the cell expresses such polypeptide sequence and recombinantly or endogenously expresses gamma secretase. Alternatively, exogenous gamma secretase, for example, soluble gamma secretase, may be added to the cell-based assay. In a series of assays, the JMD portion of Formulas I-IV can be exchanged as provided herein. For instance, using Formulas I and III, the JMD from Notch can be used in a chimeric substrate containing the TMD from APP, or vice versa. Then, using formulas II and IV, the last four residues of the JMD of this chimeric substrate can be modified to provide a different substrate. Using a single assay format, the amount of gamma secretase activity on the various substrates can be determined.

Some methods include cell-based assays wherein the chimeric JMD substrate sequences are expressed in cells that are transfected with cDNA encoding the substrate amino acid sequence. For example, some methods comprise determining whether a compound selectively inhibits gamma secretase activity at a first gamma secretase substrate relative to a second gamma secretase substrate, comprising: (a) contacting a first transfected cell culture with the compound at various concentrations under conditions that allow for gamma secretase activity; (b) contacting a second transfected cell culture with the compound at various concentrations under conditions that allow for gamma secretase activity; (c) measuring AICD produced by each of the first and second transfected cell cultures at each of the various compound concentrations to generate a first dose response curve of the effect of the compound on the first transfected cell culture and a second dose response curve of the effect of the compound on the second transfected cell culture; and (d) comparing the first and second dose response curves, wherein the first transfected cell culture is transfected with a first polynucleotide encoding a first polypeptide comprising a juxtamembrane domain (JMD1) sequence and a transmembrane domain sequence (TMD1) of any of the formulas I-IV, wherein JMD1 is from a first gamma secretase substrate; and the second transfected cell culture is transfected with a second polynucleotide encoding a second polypeptide comprising a second juxtamembrane domain (JMD2) sequence and a transmembrane domain sequence (TMD1), wherein JMD2 is from a second gamma secretase substrate and the TMD1 of the first and second polypeptides is the same. When there is a shift in the second dose response curve toward a higher concentration relative to the first dose response curve (see, e.g., the Examples included herein), it indicates that the compound is selective for the first gamma secretase substrate relative to the second gamma secretase substrate. As generally used herein, a “dose response curve shift” for any given inhibitor compound means that the IC₅₀ value of the compound has increased or decreased as a function to the gamma secretase substrate that is being tested. The IC₅₀ value of the compound for cell 1 expressing substrate 1 and cell 2 expressing substrate 2 (etc.) can be calculated from the inhibitor dose-response curves by anyone skilled in the art with or without use of various readily available computer software programs (e.g., GraphPad PRISM, MS Excel, SigmaPlot, etc.).

Some methods comprise a first gamma secretase substrate comprising a sequence from APP, and a second gamma secretase substrate comprising a sequence from APLP2, Notch, erbB4, tyrosinase, p75 NTFR, SCNB2, n-cadherin, or CD44. Other methods comprise a first gamma secretase substrate comprising a [TMD1] comprising the transmembrane domain sequence from APP; and the [JMD1] and [JMD2] sequences comprise juxtamembrane domain sequences selected from APLP2, Notch, erbB4, tyrosinase, p75 NTFR, SCNB2, n-cadherin, or CD44, and wherein [JMD1] and [JMD2] are not the same.

The gamma secretase can be added to the cell cultures by any standard technique known in the art such as, for example, transfection, electroporation, or viral vector delivery of the cells with a polynucleotide encoding gamma secretase. In other embodiments, the method comprises an active gamma secretase which is endogenously and constitutively produced by the first and second cell cultures.

Another method for determining whether a compound selectively inhibits gamma secretase activity at a first gamma secretase substrate relative to a second gamma secretase substrate, comprises:
(a) contacting a first transfected cell culture with the compound at various concentrations under conditions that allow for gamma secretase activity;
(b) contacting a second transfected cell culture with the compound at various concentrations under conditions that allow for gamma secretase activity;
(c) measuring AICD produced by each of the first and second transfected cell cultures at each of the various compound concentrations to generate a first dose response curve of the effect of the compound on the first transfected cell culture and a second dose response curve of the effect of the compound on the second transfected cell culture; and
(d) comparing the first and second dose response curves,
wherein:
   the first transfected cell culture is transfected with a first polynucleotide encoding a first polypeptide comprising the formula [JMD][TMD], wherein JMD and TMD are from a first gamma secretase substrate and
   the second transfected cell culture is transfected with a second polynucleotide encoding a polypeptide comprising Formula II:

[JMDΔC4]-X1-X2-X3-X4-[TMD]
wherein
   [JMDΔC4] and [TMD] are defined as described herein; and
   X1-X2-X3-X4 is from a second gamma secretase substrate; and
a shift in the second dose response curve toward a higher concentration relative to the first dose response curve indicates that the compound is selective for the first gamma secretase substrate relative to the second gamma secretase substrate.

In some methods, the first gamma secretase substrate is from APP, and the second gamma secretase substrate is from APLP2, Notch, erbB4, tyrosinase, p75 NTFR, SCNB2, n-cadherin, or CD44.

Some methods for determining whether a compound selectively inhibits gamma secretase activity at a first gamma secretase substrate relative to a second gamma secretase substrate, comprise:
(a) contacting a first transfected cell culture with the compound at various concentrations under conditions that allow for gamma secretase activity;
(b) contacting a second transfected cell culture with the compound at various concentrations under conditions that allow for gamma secretase activity;
(c) measuring AICD produced by each of the first and second transfected cell cultures at each of the various compound concentrations to generate a first dose response curve of the effect of the compound on the first transfected cell culture and a second dose response curve of the effect of the compound on the second transfected cell culture; and
(d) comparing the first and second dose response curves,
wherein:
   the first transfected cell culture is transfected with a polynucleotide encoding a first polypeptide comprising Formula II:

[JMDΔC4]-X1-X2-X3-X4-[TMD]
wherein
   [JMDΔC4] and [TMD] are defined as above for Formula II; and
X1-X2-X3-X4 are independently selected from any amino acid; and the second transfected cell culture is transfected with a second polynucleotide encoding a second polypeptide comprising Formula II:

[JMDΔC4]-X1-X2-X3-X4-[TMD]
wherein [TMD] and [JMDΔC4] are as defined above, and
   X1-X2-X3-X4 are independently selected from any amino acid; and
a shift in the second dose response curve toward a higher concentration relative to the first dose response curve indicates that the compound is selective for the first gamma secretase substrate relative to the second gamma secretase substrate.

In a particular embodiment X1 is selected from S, T, G, P, Q, R, V, L, N, P, A, K, E, I, F, H, W, and D; X2 is any amino acid; X3 is selected from S, N, D, P, E, R, T, F, I, K, L, V, G, W, H, and A; and X4 is any amino acid.

In a particular embodiment X1 is selected from S, T, G, P, Q, R, V, L, N, P, A, K, E, I, F, H, W, and D; X3 is selected from S, N, D, P, E; R, T, F, I, K, L, V, G, W, H, and A; and X2 and X4 are selected from L, I, H, E, V, A, S, T, D, N, P, K, Q, and R.

In a particular embodiment X1 is selected from S, T, G, P, Q, R, and D; X2 is any amino acid; X3 is selected from S, N, D, P, and A; and X4 is any amino acid.

In one embodiment of this aspect, X1-X2-X3-X4 of the first polypeptide is from a first gamma secretase substrate, and X1-X2-X3-X4 of the second polypeptide is from a second gamma secretase substrate.

In some of such methods, X1-X2-X3-X4 of the first and second polypeptide are independently selected from GLNK, SLSS, GSNK, GSNS, PPAQ, SSNK, GSSK, QHAR, QASR, TTDN, RDST, DVDR, QIPE, or DRSR, and are not the same sequence. In some methods, [TMD] of the first and second polypeptide comprises SEQ ID NO:13. In some methods, [JMDΔC4] of the first and second polypeptide are independently selected from any of SEQ ID NOs: 3-12.

In other such methods [JMDΔC4]-X1-X2-X3-X4-[TMD] comprises a sequence selected from the group consisting of:

(a) (C99GVP-APLP2):
(SEQ ID NO: 16)
LEDAEFRHDS GLEEERESVG PLREDFSLSS GAIIGLMVGG
VVIATVIVIT LVML;
(b) (C99GVP-NOTCH1):
(SEQ ID NO: 17)
LEDAEFRHDS GPYKIEAVQS ETVEPPPPAQ GAIIGLMVGG
VVIATVIVIT LVML;
(c) (C99GVP-SREBP1):
(SEQ ID NO: 18)
LEDAEFRHDS GAKPEQRPSL HSRGMLDRSR GAIIGLMVGG
VVIATVIVIT LVML;
(d) (C99APP04-APLP2):
(SEQ ID NO: 42)
LEDAEFRHDS GYEVHHQKLV FFAEDVSLSS GAIIGLMVGG
VVIATVIVIT LVML;
(e) (C99-APP-(G25S):
(SEQ ID NO: 43)
LEDAEFRHDS GYEVHHQKLV FFAEDVSSNK GAIIGLMVGG
VVIATVIVIT LVML
(f) C99-APP-(S26L):
(SEQ ID NO: 44)
LEDAEFRHDS GYEVHHQKLV FFAEDVGLNK GAIIGLMVGG
VVIATVIVIT LVML
(g) C99-APP-(N27S):
(SEQ ID NO: 45)
LEDAEFRHDS GYEVHHQKLV FFAEDVGSSK GAIIGLMVGG
VVIATVIVIT LVML
(h) C99-APP-(K28S):
(SEQ ID NO: 46)
LEDAEFRHDS GYEVHHQKLV FFAEDVGSNS GAIIGLMVGG
VVIATVIVIT LVML
(i) (C99APPA4-NOTCH1):
(SEQ ID NO: 100)
LEDAEFRHDS GYEVHHQKLV FFAEDVPPAQ GAIIGLMVGG
VVIATVIVIT LVML;
(0 (C99APPM-SREBP1):
(SEQ ID NO: 101)
LEDAEFRHDS GYEVHHQKLV FFAEDVDRSR GAIIGLMVGG
VVIATVIVIT LVML;
(k) (C99GVP-APLP2-gsnk):
(SEQ ID NO: 19)
LEDAEFRHDS GLEEERESVG PLREDFGSNK GAIIGLMVGG
VVIATVIVIT LVML;
(i) (C99GVP-NOTCH1-gsnk):
(SEQ ID NO: 20)
LEDAEFRHDS GPYKIEAVQS ETVEPPGSNK GAIIGLMVGG
VVIATVIVIT LVML;
and
(m) (C99GVP-SREBP1-gsnk):
(SEQ ID NO: 21)
LEDAEFRHDS GAKPEQRPSL HSRGMLGSNK GAIIGLMVGG
VVIATVIVIT LVML.

Using similar assays, the effect of the various candidate gamma secretase inhibitor compounds on the activity of gamma secretase can be determined for the various gamma secretase substrates. These assays include (a) contacting a polypeptide sequence of Formulas III and IV with gamma secretase and a gamma secretase inhibitor under conditions that allow for gamma secretase activity; and (b) determining the potency of the compound for inhibiting gamma secretase cleavage of the polypeptide by measuring the amount of AGBP¹ or AGBP² generated in step (a). Using these assays, compounds can be screened for their ability to inhibit gamma secretase activity at either the gamma or epsilon cleavage sites of Formula III and IV.

In order to determine the potency of the various gamma secretase inhibitors, the ability to inhibit the cleavage of gamma secretase on a natural substrate can be compared to the compound's ability to inhibit cleavage of one or more of Formulas I-IV. This assay includes contacting a naturally occurring gamma secretase substrate, fragment thereof having a naturally occurring JMD and TMD from the same naturally occurring substrate, with gamma secretase and a candidate gamma secretase inhibitor compound under conditions that allow for gamma secretase activity; and subsequently determining the potency of the compound for inhibiting gamma secretase cleavage by measuring the amount of ICD generated by the contacting step. The naturally occurring gamma secretase substrate, or fragment thereof, can comprise both (γ and ε) sites at which gamma secretase cleaves the substrate, and optionally other cleavage sites.

Some methods include identifying and/or determining the selectivity of a candidate gamma secretase inhibitor compound by comparing the potency of the compound for inhibiting gamma and epsilon cleavage of the polypeptide of Formulas I-IV. Other methods include identifying and/or determining the selectivity of a candidate gamma secretase inhibitor compound for a particular gamma secretase substrate by comparing the potency of the compound for inhibiting gamma secretase cleavage that produces ICD in a polypeptide of the invention and a naturally occurring gamma secretase substrate, or fragment thereof. As noted above, in the naturally occurring sequences, or fragments thereof, can comprise both the gamma and epsilon sites at which gamma secretase cleaves of the substrate sequence.

The methods and assays of the invention are useful for identifying gamma secretase inhibitors that are selective for APP relative to other gamma substrates (such as Notch). The methods and assays of the invention can be used to identify gamma inhibitors having IC₅₀ values ranging from about 0.01 pM-100 μM, 0.01 nM-10 μM, 0.01 nM-1 μM, 0.05 nM-100 nM, 0.07 nM-10 nM, 0.09 nM-1 nM or 0.1 nM-0.5 nM. The candidate compound can be said to be selective when the potency of inhibition by the candidate compound of ICD generation from a first gamma secretase substrate is at least about 10-fold different than ICD generation from a second gamma secretase substrate. Preferred inhibitors of gamma secretase include compounds that inhibit a gamma secretase substrate from APP with an IC₅₀ of at least about 0.05 nM or lower and inhibit such substrate from APP with an IC₅₀ at least 10-fold less than that for inhibition of a gamma secretase substrate from Notch. Thus, preferred inhibitors include compounds with an inhibitory activity to APP with an IC₅₀ of at least about 0.05 nM or lower, and a Notch IC₅₀ of at least about 0.5 nM or greater

Some methods comprise: (a) a polypeptide of Formulas I-IV and separately, a naturally occurring gamma secretase substrate sequence, for example a polypeptide that includes the JMD and TMD from the same gamma secretase substrate; (b) contacting the polypeptides of (a) with a candidate compound selective for gamma secretase inhibition under conditions that allow for gamma secretase activity; (c) measuring the amount of ICD generated from the contacting in step (b); and (d) determining the selectivity of the candidate compound; wherein the candidate Compound is determined to be selective when the potency of inhibition of ICD generation in step (c) for the polypeptide of SEQ ID NO:1 is increased or decreased from the level of ICD measured from the naturally occurring gamma secretase substrate sequence. In some methods, the naturally occurring gamma secretase substrate sequence comprises the JMD and TMD from APP.

In general, the measuring step (c) can employ any method that is effective for detecting the amount of ICD generated by gamma secretase. For example, reporter genes can be activated by the GVP sequence and used monitor the amount of ICD generated by gamma secretase cleavage. Specific binding agents can be employed in this assay generally, for detection of both ICD and beta peptides In an embodiment of this aspect the measuring step (c) comprises contacting the ICD with a specific binding agent for ICD. In some methods, the measuring step (c) comprises contacting the ICD with two specific binding agents for two different epitopes of ICD, such as two antibodies as used in a sandwich ELISA assay. The measuring step (c) can comprise a reporter molecule and/or reporter gene, such as, for example, a luciferase reporter system.

The ICD fragment can be derived from any γ-secretase substrate such as, for example, APP, Notch1, APLP2, erbB4, tyrosinase, p75 NTFR, SCNB2, n-cadherin, CD44, as well as any other transmembrane protein(s) having at least one gamma secretase cleavage site located within its transmembrane region.

In some methods, the specific binding agent comprises an antibody for ICD, such as, for example, a monoclonal antibody that specifically binds APP-ICD (AICD) or Notch-ICD (NICD). Antibodies can be generated to an ICD or fragment thereof and can be used with the assay. Some polyclonal AICD neoepitope antibodies (polyclonal #66104) against an antigenic peptide have been described (Kimberly, W. T., et al., Biochemistry; (2003); 42(1):137-144). One antigenic peptide for generating a monoclonal or polyclonal antibody that specifically binds AICD has the amino acid sequence VMLKKKC (SEQ ID NO:39). This particular sequence can be used to generate both polyclonal and monoclonal antibodies, such as, for example, the monoclonal antibody 22B11 as described herein. Accordingly, the invention provides antibodies, including monoclonal antibodies, raised to or that specifically bind the amino acid sequence VMLKKKC (SEQ ID NO:39), such as, for example, antibody 22B11. In some methods, the specific binding agent comprises an antibody raised to or that specifically binds the amino acid sequence of SEQ ID NO:39, such as, for example, antibody 22B11.

The methods are useful for determining the potency, activity, specificity, and selectivity of identified or unidentified gamma secretase inhibitor compounds. The methods are useful for determining whether structural determinants on the substrate play a role in inhibitor activity and/or selectivity. Similarly the methods are useful for determining whether certain inhibitors act primarily through inhibition of a particular gamma secretase cleavage site (e.g., yore, S2 or S3, etc.). The methods are also useful for determining whether the JMD is involved in conferring potency and selectivity for certain gamma secretase inhibitors.

Thus, the invention also provides a method for determining the potency of a gamma secretase inhibitor for inhibiting cleavage of a gamma secretase substrate by gamma secretase, the method comprising: (a) contacting a polypeptide of Formulas I-IV with gamma secretase and the gamma secretase inhibitor under conditions that allow for gamma-secretase activity, and (b) measuring the amount of gamma secretase activity. For example, the invention provides a method for determining whether a compound inhibits γ-secretase in a site-specific or a substrate specific manner comprising: (a) providing a polypeptide sequence of Formulas I-IV; (b) separately providing a polypeptide sequence from a naturally occurring γ-secretase substrate or fragment thereof containing the naturally occurring TMD and JMD from a single naturally occurring substrate; (c) contacting the polypeptide of (a) and (b) with the compound under conditions that allow for gamma secretase activity; (d) determining the amount of gamma secretase activity from the contacting step of (c) for each polypeptide; and (e) comparing the results from step (d) and determining that the compound inhibits gamma secretase in a site-specific or a substrate-specific manner, when the compound has a reduced or increased inhibition potency against gamma secretase at the ε-cleavage site of the polypeptide of Formulas I-IV, compared to the naturally occurring gamma secretase substrate.

In some methods, the compound is a site specific inhibitor of gamma secretase when the potency for inhibition of cleavage products at either of two sites from the polypeptide of Formulas I-IV in the presence of the compound is decreased or increased by an order of magnitude relative to the other of the two sites in the same substrate. In other methods, the compound is a substrate specific inhibitor of gamma secretase when the potency of inhibition of the same site, e.g. the γ- and/or ε-sites from the polypeptide of Formulas I-IV in the presence of the compound is decreased or increased by an order of magnitude or more when comparing two different substrates such that JMD1 is from substrate 1 and JMD2 is from substrate 2.

The invention provides a method for modulating the activity of gamma secretase on a gamma secretase substrate comprising introducing a modification to the amino acid sequence of the gamma secretase substrate at the four amino acid residues located immediately to the transmembrane region of the gamma secretase substrate. As noted above in the description of the gamma secretase substrate sequences, certain of the four C-terminal amino acids (X1-X4) may play a greater role in determining the specificity that gamma secretase has for a particular substrate sequence. Thus, using routine techniques known in the art, a series of mutagenesis experiments can be designed that can identify the optimal amino acid(s) for these particular sequences (e.g., X2 and X4). Thus, a particular native JMD can be selected, and a series of amino acid mutants can be made wherein all the residues except those corresponding to X2 and X4 are kept native, while residues X2 and X4 are varied using the twenty naturally occurring amino acids. An assay measuring gamma secretase activity can be used to screen the resulting mutant sequences for those which exhibit the largest change in gamma secretase activity. The modification can comprise a substitution of the four amino acid residues with four amino acids selected from the group consisting of G, N, T, S, V, H, K, L, I, P, A, Q, D, E, and R. The modification can comprise a substitution of the four amino acid residues with a sequence selected from sequence GSNK, SLSS, PPAQ, DRSR, QHAR, QASR, TTDN, RDST, DVDR, and QIPE.

Also provided is a method of modulating gamma secretase activity at the gamma and/or epsilon cleavage sites on a gamma secretase substrate comprising introducing modifications to the amino acid sequence of the juxtamembrane region of the gamma secretase substrate, wherein the modification is selected from: (a) insertion of an amino acid sequence comprising GSNK, when the gamma secretase substrate is not APP; SLSS, when the gamma secretase substrate is not APLP2; PPAQ, when the gamma secretase substrate is not Notch1; QHAR, when the gamma secretase substrate is not erbB4; QASR, when the gamma secretase substrate is not tyrosinase; TTDN when the gamma secretase substrate is not p75 NTFR; RDST, when the gamma secretase substrate is not SCNB2; DVDR, when the gamma secretase substrate is not n-cadherin; and QIPE, when the gamma secretase substrate is not CD44; and (b) substitution of the four amino acids immediately to the N-terminal side of the transmembrane region with a sequence selected from the group consisting of GSNK, SLSS, PPAQ, QHAR, QASR, TTDN, RDST, DVDR, QIPE, and DRSR, with the provisos that GNSK is not selected when the gamma secretase substrate is APP; SLSS is not selected when the gamma secretase substrate is APLP2; PPAQ is not selected when the gamma secretase substrate is Notch1; QHAR is not selected when the gamma secretase substrate is erbB4; QASR is not selected when the gamma secretase substrate is tyrosinase; TTDN is not selected when the gamma secretase substrate is p75 NTFR; RDST is not selected when the gamma secretase substrate is SCNB2; DVDR is not selected when the gamma secretase substrate is n-cadherin; and QIPE is not selected when the gamma secretase substrate is CD44.

Also provided is a method of modulating gamma secretase selectivity for a gamma secretase substrate comprising introducing modifications to the amino acid sequence of the juxtamembrane region of the gamma secretase substrate, wherein the modification is selected from: (a) insertion of an amino acid sequence comprising GSNK, when the gamma secretase substrate is not APP; SLSS, when the gamma secretase substrate is not APLP2; and PPAQ, when the gamma secretase substrate is not Notch1; and (b) substitution of the four amino acids immediately to the N-terminal side of the transmembrane region with a sequence selected from the group consisting of GSNK, SLSS, PPAQ, QHAR, QASR, TTDN, RDST, DVDR, QIPE, and DRSR, with the provisos that GNSK is not selected when the gamma secretase substrate is APP; SLSS is not selected when the gamma secretase substrate is APLP2; PPAQ is not selected when the gamma secretase substrate is Notch1; QHAR is not selected when the gamma secretase substrate is erbB4; QASR is not selected when the gamma secretase substrate is tyrosinase; TTDN is not selected when the gamma secretase substrate is p75 NTFR; RDST is not selected when the gamma secretase substrate is SCNB2; DVDR is not selected when the gamma secretase substrate is n-cadherin; and QIPE is not selected when the gamma secretase substrate is CD44.

Where substrates are of either Formulas II or IV, only the residues of X2 and X4 are modified, while residues X1 and X3 are from the naturally occurring JMD sequence, for example, as disclosed in the non-limiting sequences SEQ ID NOs:44 and 46.

Also provided is a method of predicting the selectivity of a gamma secretase inhibitor on a gamma secretase substrate, comprising analyzing the amino acid sequence of the gamma secretase substrate; comparing the amino acid sequence of the gamma secretase substrate in the JMD region with the amino acid sequence of other gamma secretase substrates; and determining how the selectivity of the gamma secretase inhibitor on the gamma secretase substrate is affected by alterations in the degree of sequence homology or identity it shares with others gamma secretase substrates.

Also provided is a polynucleotide sequence encoding the polypeptide sequence of any of Formulas I-IV, for example, a polynucleotide sequence encoding a polypeptide comprising any of SEQ ID NOs: 1-51 and 91-101.

The invention provides vectors, recombinant cells, and transgenic non-human animals comprising polynucleotide sequences encoding the polypeptide sequences of any of Formulas I-IV or of a recombinant naturally occurring gamma secretase substrate or fragment thereof, for example, recombinant cells and transgenic non-human animals comprising the polypeptide sequences of SEQ ID NOs.1, 3-12, 15-36, 42-51, and/or 94-101.

Given the amino acid sequences of the polypeptides, those of ordinary skill in the art will be able to generate polynucleotide sequences, and optimize those sequences for expression in various cell types and expression systems, using the well known genetic codes and optimized codons for various organisms and expression systems.

### Compounds, Compositions, and Methods of Treatment

In other aspects the invention provides compounds that inhibit gamma secretase in a substrate or site specific manner, pharmaceutical compositions comprising such compounds, methods of treating Alzheimer's disease using such compounds, and methods of inhibiting gamma secretase activity using such compounds.

Thus, the invention provides a compound that inhibits gamma secretase in a site specific manner. Some compounds of the invention preferentially inhibit gamma secretase activity at the gamma cleavage site of the gamma secretase substrate. Some compounds of the invention preferentially inhibit gamma secretase activity at the epsilon cleavage site of the gamma secretase substrate.

A compound that inhibits gamma secretase activity at either the gamma or the epsilon cleavage site of the gamma secretase substrate is identified by the assay method of the invention by (a) providing a polypeptide sequence of Formulas I-IV; (b) separately providing a polypeptide sequence from a naturally occurring gamma secretase substrate; (c) contacting the polypeptide of (a) and (b) with the compound under conditions that allow for gamma secretase activity; (d) determining the amount of gamma secretase activity at the gamma and epsilon cleavage sites from the contacting step of (c) for each polypeptide; (e) determining the amount of gamma secretase activity at the gamma and epsilon cleavage sites from the contacting step of (b); and (f) comparing the results from steps (d) and (e) and determining that the compound inhibits gamma secretase in a site-specific or a substrate-specific manner.

A compound selectively inhibits gamma secretase activity at the gamma cleavage site of the gamma secretase substrate when the EC₅₀ value calculated for the compound inhibitory activity at the gamma cleavage site is smaller than the EC₅₀ value calculated for the compound inhibitory activity at the epsilon cleavage site, within the same substrate, or over a number of different gamma secretase substrates. A compound is a substrate specific inhibitor of gamma secretase when the EC₅₀ value calculated for the compound inhibitory activity at a given site, e.g. the epsilon cleavage site of the substrate (or sequence comprising the JMD of that substrate), is smaller than the EC₅₀ value calculated for the compound inhibitory activity at the equivalent site, e.g. the epsilon cleavage site, over a number of different gamma secretase substrates (that do not comprise the same JMD sequence). Some compounds comprise a sulfonamide functional group.

Also provided is a compound that can be identified by the methods provided herein that selectively inhibits cleavage of a first gamma secretase substrate selected from amyloid precursor protein (APP), Notch, amyloid precursor-like protein (APLP2), tyrosinase, CD44, erbB4, p75 NTFR, n-cadherin and SCNB2 relative to at least one different gamma secretase substrate selected from APP, Notch, APLP2, SREBP1, tyrosinase, CD44, erbB4, p-75 NTFR, n-cadherin and SCNB2. Some compounds selectively inhibit cleavage of APP relative to at least one gamma secretase substrate selected from Notch, APLP2, tyrosinase, CD44, erbB4, p-75 NTFR, n-cadherin and SCNB2. Some compounds selectively inhibit cleavage of APP relative to at least one gamma secretase substrate selected from Notch and APLP2.

The invention provides compositions comprising the above-described compounds, in combination with a pharmaceutically acceptable salt, vehicle, carrier, diluent, and/or adjuvant.

The compounds can be administered orally, parenterally, (IV, IM, depo-IM, SQ, and depo SQ), sublingually, intranasally (inhalation), intrathecally, topically, or rectally. Dosage forms known to those of skill in the art are suitable for delivery of the compounds of the invention.

Compositions are provided that contain therapeutically effective amounts of the compounds of the invention. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

About 1 to 500 mg of a compound or mixture of compounds of the invention or a physiologically acceptable salt or ester can be compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 2 to about 100 mg, more preferably about 10 to about 30 mg of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

To prepare compositions, one or more compounds of the invention are mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

Pharmaceutical carriers or vehicles suitable for administration of the compounds provided herein include any such carriers known to those skilled in the art to be suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

Methods for solubilizing can be used when the compounds exhibit insufficient solubility for effective formulation. Such methods are known and include, but are not limited to, using cosolvents such as dimethylsulfoxide (DMSO), using surfactants such as Tween®, and dissolution in aqueous sodium bicarbonate. Derivatives of the compounds, such as salts or prodrugs may also be used in formulating effective pharmaceutical compositions.

The concentration of the compound is effective for delivery of an amount upon administration that lessens or ameliorates at least one symptom of the disorder for which the compound is administered. Typically, the compositions are formulated for single dosage administration.

The compounds of the invention may be prepared with carriers that protect them against rapid elimination from the body, such as time-release formulations or coatings. Such carriers include controlled release formulations, such as, but not limited to, microencapsulated delivery systems. The active compound is included in the pharmaceutically acceptable carrier in an amount sufficient to exert a therapeutically useful effect in the absence of undesirable side effects on the subject treated. The therapeutically effective concentration may be determined empirically by testing the compounds in known in vitro and in vivo model systems for the treated disorder.

The compounds and compositions of the invention can be enclosed in multiple or single dose containers. The enclosed compounds and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the compound of the invention. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The concentration of active compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a glidant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. Syrups can contain, in addition to the active compounds, sucrose as a sweetening agent and certain preservatives, dyes and colorings, and flavors.

The active materials can also be mixed with other active materials that do not impair the desired action, or with materials that supplement the desired action.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known in the art, for example, as described in U.S. Pat. No. 4,522,811.

The active compounds may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

Compounds of the invention may be administered enterally or parenterally. When administered orally, compounds of the invention can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, it is preferred that they be of the sustained release type so that the compounds of the invention need to be administered only once or twice daily.

The oral dosage forms are administered to the subject 1, 2, 3, or 4 times daily. It is preferred that the compounds of the invention be administered either three or fewer times, more preferably once or twice daily. Hence, it is preferred that the compounds of the invention be administered in oral dosage form. It is preferred that whatever oral dosage form is used, that it be designed so as to protect the compounds of the invention from the acidic environment of the stomach. Enteric coated tablets are well known to those skilled in the art. In addition, capsules filled with small spheres each coated to protect from the acidic stomach, are also well known to those skilled in the art.

As noted above, depending on whether asymmetric carbon atoms are present, the compounds of the invention can be present as mixtures of isomers, as racemates, or in the form of pure isomers.

Salts of compounds are preferably the pharmaceutically acceptable or non-toxic salts. For synthetic and purification purposes it is also possible to use pharmaceutically unacceptable salts.

The composition can comprise an additional agent effective for the treatment of Alzheimer's disease, as are known in the art.

Also provided are methods of treating and/or preventing a disease associated with the deposition of amyloid beta peptide, such as, for example, Alzheimer's disease or Mild Cognitive Impairment, in a subject in need of such treatment, comprising administering to the subject an effective amount of a compound, or salt thereof, identified by the assay method of the invention. Some methods can help prevent, delay or slow the development or progression of Alzheimer's disease. In some methods, the subject has been diagnosed with Alzheimer's disease. In preferred such methods the subject is human.

Similarly the invention provides methods of treating and/or preventing a disease associated with activation of Notch signaling such as, for example, cancer and autoimmune diseases, in a subject in need of such treatment, comprising administering to the subject an effective amount of a compound, or salt thereof, identified by the assay method of the invention. Some methods can help prevent, delay or slow the development or progression of cancer or an autoimmune disease. In some methods, the subject has been diagnosed with cancer or an autoimmune disease. In preferred such methods the subject is human.

The methods of treatment employ therapeutically effective amounts: for oral administration from about 0.1 mg/day to about 1,000 mg/day; for parenteral, sublingual, intranasal, intrathecal administration from about 0.5 to about 100 mg/day; for depo administration and implants from about 0.5 mg/day to about 50 mg/day; for topical administration from about 0.5 mg/day to about 200 mg/day; for rectal administration from about 0.5 mg to about 500 mg.

Therapeutically effective amounts for oral administration can be from about 1 mg/day to about 100 mg/day, preferably mg/day to about 50 mg/day; and for parenteral administration from about 5 to about 50 mg daily.

The invention also provides a method of selectively inhibiting gamma secretase activity on a particular substrate, or gamma secretase activity at a particular cleavage site of a substrate in a cell, comprising contacting a cell with a compound identified by the assay of the invention effective to selectively inhibit gamma secretase. Some methods inhibit gamma secretase activity by about three- to five-fold relative to normal activity. Even more preferably, the method inhibits gamma secretase activity by about five-fold to about ten-fold, more preferably by about ten-fold to fifteen-fold, and yet more preferably, by about fifteen-fold to about twenty-fold over normal activity. Yet even more preferably, the method inhibits gamma secretase activity by more than about twenty-fold. The cell can be a mammalian cell, such as, for example, a human cell. In some methods, the cell is an isolated mammalian cell, preferably an isolated human cell.

A method of selectively inhibiting gamma secretase at either the gamma or epsilon cleavage site of a given gamma secretase substrate, can be used to treat a subject that has a disease or a disorder related to activity of gamma secretase at either the gamma or epsilon cleavage site against said substrate. In some methods, the subject demonstrates clinical signs of a disease or a disorder related to gamma secretase activity at one or the other of gamma or epsilon cleavage sites of a given gamma secretase substrate. In some methods, the subject is diagnosed with a disease or a disorder related to disregulated activity of gamma secretase against a given substrate. Some diseases or disorders relate to gamma secretase activity at the gamma cleavage site and not gamma secretase activity at the epsilon cleavage site. As the compounds useful in this method are identified by the assay of the invention as selective inhibitors of gamma secretase substrates or gamma secretase cleavage sites of gamma secretase substrates, methods of treating disorders or diseases related to gamma secretase can be treated without adversely effecting gamma secretase activity on other gamma secretase substrates, or at other cleavage sites (e.g., such as Notch signaling, or cleavage at the epsilon cleavage site of gamma secretase substrates).

The methods and assay of the invention can employ any type of assay known in the art that can determine the amount of beta peptide and ICD in a cell. In one embodiment the assay is any type of binding assay, preferably an immunological binding assay. Such immunological binding assays are well known in the art (see, Asai, ed., Methods in Cell Biology, Vol. 37, Antibodies in Cell Biology, Academic Press, Inc., New York (1993)). Immunological binding assays typically utilize a capture agent to bind specifically to and often immobilize the analyte target antigen. The capture agent can be a moiety that specifically binds to the analyte. The capture agent can be an antibody or fragment thereof that specifically binds Aβ, such as, for example, an antibody or fragment thereof that specifically binds to an epitope located in the forty amino acid residues of Aβ. Some such antibodies or fragments thereof specifically bind to an epitope located in the first 23 amino acid residues of Aβ (i.e., Aβ1-23). Some antibodies or fragments thereof specifically bind to an epitope of a fragment generated from cleavage by gamma secretase at a gamma secretase substrate, such as, for example, an antibody or fragment thereof that specifically binds to an epitope of an ICD peptide generated from a gamma secretase substrate. Some of these agents are commercially available (APP C-terminal antibody for Sigma Aldrich, Cat. #A8717), and some such agents can be generated using standard immunogenic techniques (e.g., hybridoma, anti-sera, polyclonal antibody generation).

Immunological binding assays frequently utilize a labeling agent that will signal the existence of the bound complex formed by the capture agent and antigen. The labeling agent can be one of the molecules comprising the bound complex; i.e. it can be labeled specific binding agent or a labeled anti-specific binding agent antibody. Alternatively, the labeling agent can be a third molecule, commonly another antibody, which binds to the bound complex. The labeling agent can be, for example, an anti-specific binding agent antibody bearing a label. The second antibody, specific for the bound complex, may lack a label, but can be bound by a fourth molecule specific to the species of antibodies which the second antibody is a member of. For example, the second antibody can be modified with a detectable moiety, such as biotin, which can then be bound by a fourth molecule, such as enzyme-labeled streptavidin. Other proteins capable of specifically binding immunoglobulin constant regions, such as protein A or protein G may also be used as the labeling agent. These binding proteins are normal constituents of the cell walls of streptococcal bacteria and exhibit a strong non-immunogenic reactivity with immunoglobulin constant regions from a variety of species (see, generally Akerstrom, J Immunol, 135:2589-2542 (1985); and Chaubert, Mod Pathol, 10:585-591 (1997)). The labeling agent can comprise an antibody or fragment thereof that specifically binds the first twenty-three amino acid residues of Aβ (Aβ1-23). Some such antibodies or fragments thereof specifically bind to an epitope located in the first 7 amino acid residues of Aβ (i.e., Aβ1-7), and some such antibodies or fragments thereof specifically bind to an epitope located in the first 5 amino acid residues of Aβ (i.e., Aβ1-5).

Throughout the assays, incubation and/or washing steps may be required after each combination of reagents. Incubation steps can vary from about 5 seconds to several hours, preferably from about 5 minutes to about 24 hours. However, the incubation time will depend upon the assay format, analyte, volume of solution, concentrations, and the like. Usually, the assays will be carried out at ambient temperature, although they can be conducted over a range of temperatures.

Assays that demonstrate inhibition of either site specific or substrate specific gamma secretase-mediated cleavage can utilize any of the known forms of gamma secretase substrates, including the large number of APP forms, such as the non-limiting examples of the 695 amino acid “normal” isotype described by Kang et al., 1987, Nature 325:733-6, the 770 amino acid isotype described by Kitaguchi et. al., 1981, Nature 331:530-532, and variants such as the Swedish Mutation (KM670-1NL) (APPswe), the London Mutation (V7176F), and others. See, for example, U.S. Pat. No. 5,766,846 and also Hardy, 1992, Nature Genet. 1:233-234, for a review of known variant mutations. Additional useful substrates include the dibasic amino acid modification, APP-KK disclosed, for example, in WO 00/17369, fragments of APP, and synthetic peptides containing the gamma-secretase cleavage site, wild type (WT) or mutated form, e.g., APPswe, as described, for example, in U.S. Pat. Nos. 5,441,870, 5,605,811, 5,721,130, 6,018,024, 5,604,102, 5,612,486, 5,850,003, and 6,245,964.

Immunological binding assays can be of the non-competitive type. These assays have an amount of captured analyte that is directly measured. For example, in one preferred “sandwich” assay, the capture agent (antibody) can be bound directly to a solid substrate where it is immobilized. These immobilized antibodies then capture (bind to) antigen present in the test sample. The protein thus immobilized is then bound to a labeling agent, such as a second antibody having a label. In another contemplated “sandwich” assay, the second antibody lacks a label, but can be bound by a labeled antibody specific for antibodies of the species from which the second antibody is derived. The second antibody also can be modified with a detectable moiety, such as biotin, to which a third labeled molecule can specifically bind, such as streptavidin. (See, Harlow and Lane, Antibodies, A Laboratory Manual, Ch 14, Cold Spring Harbor Laboratory, NY (1988), incorporated herein by reference).

Immunological binding assays can be of the competitive type. The amount of analyte present in the sample is measured indirectly by measuring the amount of an added analyte displaced, or competed away, from a capture agent by the analyte present in the sample. In one preferred competitive binding assay, a known amount of analyte, usually labeled, is added to the sample and the sample is then contacted with an antibody (the capture agent). The amount of labeled analyte bound to the antibody is inversely proportional to the concentration of analyte present in the sample. (See, Harlow and Lane, Antibodies, A Laboratory Manual, Ch 14, pp. 579-583, supra).

In another contemplated competitive binding assay, the antibody is immobilized on a solid substrate. The amount of protein bound to the antibody may be determined either by measuring the amount of protein present in a protein/antibody complex, or alternatively by measuring the amount of remaining uncomplexed protein. The amount of protein may be detected by providing a labeled protein. See, Harlow and Lane, Antibodies, A Laboratory Manual, Ch 14, supra).

In yet another contemplated competitive binding assay, hapten inhibition is utilized. Here, a known analyte is immobilized on a solid substrate. A known amount of antibody is added to the sample, and the sample is contacted with the immobilized analyte. The amount of antibody bound to the immobilized analyte is inversely proportional to the amount of analyte present in the sample. The amount of immobilized antibody may be detected by detecting either the immobilized fraction of antibody or the fraction that remains in solution. Detection may be direct where the antibody is labeled or indirect by the subsequent addition of a labeled moiety that specifically binds to the antibody as described above.

The competitive binding assays can be used for cross-reactivity determinations to permit a skilled artisan to determine if a protein or enzyme complex which is recognized by a specific binding agent of the invention is the desired protein and not a cross-reacting molecule or to determine whether the antibody is specific for the antigen and does not bind unrelated antigens. In assays of this type, antigen can be immobilized to a solid support and an unknown protein mixture is added to the assay, which will compete with the binding of the specific binding agents to the immobilized protein. The competing molecule also binds one or more antigens unrelated to the antigen. The ability of the proteins to compete with the binding of the specific binding agents/antibodies to the immobilized antigen is compared to the binding by the same protein that was immobilized to the solid support to determine the cross-reactivity of the protein mix.

Other non-immunologic techniques for detecting beta and ICD peptides which do not require the use of beta- and ICD-specific antibodies may also be employed. For example, two-dimensional gel electrophoresis may be employed to separate closely related soluble proteins present in a fluid sample. Antibodies which are cross-reactive with many fragments of beta and/or ICD polypeptides, for example, Aβ, may then be used to probe the gels, with the presence of the particular peptide being identified based on its precise position on the gel. In the case of cultured cells, the cellular proteins may be metabolically labeled and separated by SDS-polyacrylamide gel electrophoresis, optionally employing immunoprecipitation as an initial separation step.

The present invention also provides Western blot methods to detect or quantify the presence of Aβ and/or ICDs in a sample. The technique generally comprises separating sample proteins by gel electrophoresis on the basis of molecular weight and transferring the proteins to a suitable solid support, such as nitrocellulose filter, a nylon filter, or derivatized nylon filter. The sample is incubated with antibodies or fragments thereof that specifically bind Aβ and/or ICDs and the resulting complex is detected. These antibodies may be directly labeled or alternatively may be subsequently detected using labeled antibodies that specifically bind to the antibody.

### Binding Reagents

The method of the invention can comprise a specific binding agent to a beta peptide, such as, for example, an antibody to Aβ. When the method comprises at least two antibodies to Aβ, one antibody preferably acts as a “capture” molecule, while the other antibody acts as the detection or “labeled” molecule. In certain embodiments the capture antibody can recognize an epitope of Aβ, for example, the capture antibody preferably recognizes an epitope within amino acids 1-28.

Products characteristic of APP cleavage can be measured by immunoassay using various antibodies such as those as described, for example, in Pirttila et al., 1999, Neuro. Lett. 249:21-4, and in U.S. Pat. No. 5,612,486. Useful antibodies to detect Aβ include, for example, the monoclonal antibody 6E10 (Senetek, St. Louis, Mo.) that specifically recognizes an epitope on amino acids 1-16 of the Aβ peptide; antibodies 162 and 164 (New York State Institute for Basic Research, Staten Island, N.Y.) that are specific for human Aβ 1-40 and 1-42, respectively; and antibodies that recognize the junction region of beta-amyloid peptide, the site between residues 16 and 17, as described in U.S. Pat. No. 5,593,846. Antibodies raised against a synthetic peptide of residues 591 to 596 of APP and SW192 antibody raised against 590-596 of the Swedish mutation are also useful in immunoassay of APP and its cleavage products, as described in U.S. Pat. Nos. 5,604,102 and 5,721,130. Thus, antibodies specific for regions of gamma secretase substrates, such as Aβ, ICD, TMD, and C-terminal regions can be prepared against a suitable antigen or hapten comprising the desired target epitope, such as (for APP) amino acids 4-7 (A-beta), the junction region consisting of amino'acid residues 13-28, amino acids 33-40 (specific for Aβ₄₀), amino acids 30-42 (specific for Aβ₄₂), amino acids 50-55 (AICD N-terminus), and the C-terminal portion of APP. Conveniently, synthetic peptides may be prepared by conventional solid phase techniques, coupled to a suitable immunogen, and used to prepare antisera or monoclonal antibodies by conventional techniques. Suitable peptide haptens will usually comprise at least five contiguous residues within Aβ and may include more than six residues.

Synthetic polypeptide haptens may be produced by the well-known Merrifield solid-phase synthesis technique in which amino acids are sequentially added to a growing chain (Merrifield, J. Am. Chem. Soc., (1963); 85:2149-2156). The amino acid sequences may be based on the sequences of the ICDs or N-terminal fragments of known gamma secretase substrates that are known in the art and/or discussed specifically herein.

Once a sufficient quantity of polypeptide hapten has been obtained, it may be conjugated to a suitable immunogenic carrier, such as serum albumin, keyhole limpet hemocyanin, or other suitable protein carriers, as generally described in Hudson and Hay, Practical Immunology, Blackwell Scientific Publications, Oxford, Chapter 1.3, 1980, the disclosure of which is incorporated herein by reference. An exemplary immunogenic carrier that has been useful is αCD3κ antibody (Boehringer-Mannheim, Clone No. 145-2C11).

Once a sufficient quantity of the immunogen has been obtained, antibodies specific for the desired epitope may be produced by in vitro or in vivo techniques. In vitro techniques involve exposure of lymphocytes to the immunogens, while in vivo techniques require the injection of the immunogens into a suitable vertebrate host. Suitable vertebrate hosts are non-human, including mice, rats, rabbits, sheep, goats, and the like. Immunogens are injected into the animal according to a predetermined schedule, and the animals are periodically bled, with successive bleeds having improved titer and specificity. The injections may be made intramuscularly, intraperitoneally, subcutaneously, or the like, and an adjuvant, such as incomplete Freund's adjuvant, may be employed.

If desired, monoclonal antibodies can be obtained by preparing immortalized cell lines capable of producing antibodies having desired specificity. Such immortalized cell lines may be produced in a variety of ways. Conveniently, a small vertebrate, such as a mouse is hyperimmunized with the desired immunogen by the method just described. The vertebrate is then killed, usually several days after the final immunization, the spleen cells removed, and the spleen cells immortalized. The manner of immortalization is not critical. Monoclonal antibodies useful in the invention may be made by the hybridoma method as described in Kohler et al., Nature 256:495 (1975); the human B-cell hybridoma technique (Kosbor et al., Immunol Today 4:72 (1983); Cote et al., Proc Natl Acad Sci (USA) 80: 2026-2030 (1983); Brodeur et al., Monoclonal Antibody Production Techniques and Applications, pp. 51-63, Marcel Dekker, Inc., New York, (1987)) and the EBV-hybridoma technique (Cole et al., Monoclonal Antibodies and Cancer Therapy, Alan R Liss Inc, New York N.Y., pp 77-96, (1985)).

When the hybridoma technique is employed, myeloma cell lines can be used. Such cell lines suited for use in hybridoma-producing fusion procedures preferably are non-antibody-producing, have high fusion efficiency, and enzyme deficiencies that render them incapable of growing in certain selective media which support the growth of only the desired fused cells (hybridomas). For example, cell lines used in mouse fusions are Sp-20, P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; cell lines used in rat fusions are R210.RCY3, Y3-Ag 1.2.3, IR983F and 4B210. Other cell lines useful for cell fusions are U-266, GM1500-GRG2, LICR-LON-HMy2 and UC729-6. Hybridomas and other cell lines that produce monoclonal antibodies are contemplated to be novel compositions of the present invention.

The phage display technique may also be used to generate monoclonal antibodies from any species. Preferably, this technique is used to produce fully human monoclonal antibodies in which a polynucleotide encoding a single Fab or Fv antibody fragment is expressed on the surface of a phage particle. (Hoogenboom et al., J Mol Biol 227: 381 (1991); Marks et al., J Mol Biol 222: 581 (1991); see also U.S. Pat. No. 5,885,793)). Each phage can be “screened” using binding assays described herein to identify those antibody fragments having affinity for Aβ and/or ICDs. Thus, these processes mimic immune selection through the display of antibody fragment repertoires on the surface of filamentous bacteriophage, and subsequent selection of phage by their binding to Aβ and/or ICDs. One such procedure is described in PCT Application No. PCT/US98/17364, filed in the name of Adams et al., which describes the isolation of high affinity and functional agonistic antibody fragments for MPL- and msk-receptors using such an approach. In this approach, a complete repertoire of human antibody genes can be created by cloning naturally rearranged human V genes from peripheral blood lymphocytes as previously described (Mullinax et al., Proc Natl Acad Sci (USA) 87: 8095-8099 (1990)). Specific techniques for preparing monoclonal antibodies are described in Antibodies: A Laboratory Manual, Harlow and Lane, eds., Cold Spring Harbor Laboratory, 1988, the full disclosure of which is incorporated herein by reference.

In addition to monoclonal antibodies and polyclonal antibodies (antisera), the detection techniques of the present invention will also be able to use antibody fragments, such as F(ab), Fv, V_{L}, V_{H}, and other fragments. In the use of polyclonal antibodies, however, it may be necessary to adsorb the anti-sera against the target epitopes in order to produce a monospecific antibody population. It will also be possible to employ recombinantly produced antibodies (immunoglobulins) and variations thereof as now well described in the patent and scientific literature. See, for example, EPO 8430268.0; EPO'85102665.8; EPO 85305604.2; PCT/GB 85/00392; EPO 85115311.4; PCT/US86/002269; and Japanese application 85239543, the disclosures of which are incorporated herein by reference. It would also be possible to prepare other recombinant proteins which would mimic the binding specificity of antibodies prepared as just described.

The cell types that can be used with the invention include any type of cell, either naturally occurring or artificially constructed, that express a gamma-secretase substrate comprising SEQ ID NO.1, and that allow for gamma secretase activity. Non-limiting examples include the types of cells discussed herein, including those in the Examples. Using known methods, or those disclosed herein, one of skill can transform/transfect such cells with a cDNA encoding for a gamma secretase substrate comprising a polypeptide comprising SEQ ID NO.1, and a wild-type gamma secretase substrate, either sequentially or at the same time. Any known methods of recombinant nucleic acid technology, genetic manipulation (i.e., creating knockout strains), and cell transformation/transfection can be used, as well as those methods as described in detail herein.

Standard techniques may be used for recombinant DNA molecule, protein, and antibody production, as well as for tissue culture and cell transformation. See, e.g., Sambrook, et al. (below) or Current Protocols in Molecular Biology (Ausubel et al., eds., Green Publishers Inc. and Wiley and Sons 1994). Enzymatic reactions and purification techniques are typically performed according to the manufacturer's specifications or as commonly accomplished in the art using conventional procedures such as those set forth in Sambrook et al. (Molecular Cloning: A Laboratory Manual. Cold Spring Harbor Laboratory Press, Cold Spring Harbor, N.Y. (1989)), or as described herein. Unless specific definitions are provided, the nomenclature utilized in connection with, and the laboratory procedures and techniques of analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, and delivery, and treatment of patients.

It should be noted that the section headings are used herein for organizational purposes only, and are not to be construed as in any way limiting the subject matter described. All references cited herein are incorporated by reference in their entirety.

The Examples that follow are merely illustrative of specific embodiments of the invention, and are not to be taken as limiting the invention, which is defined by the appended claims.

### EXAMPLES

### General Techniques

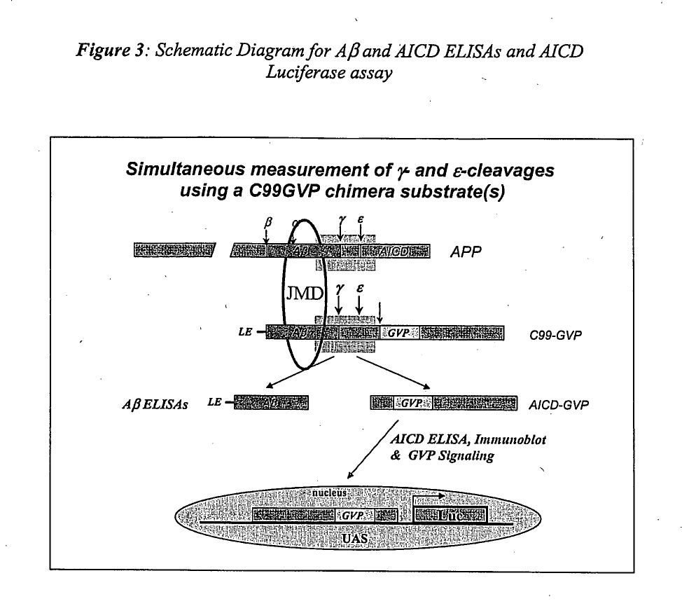

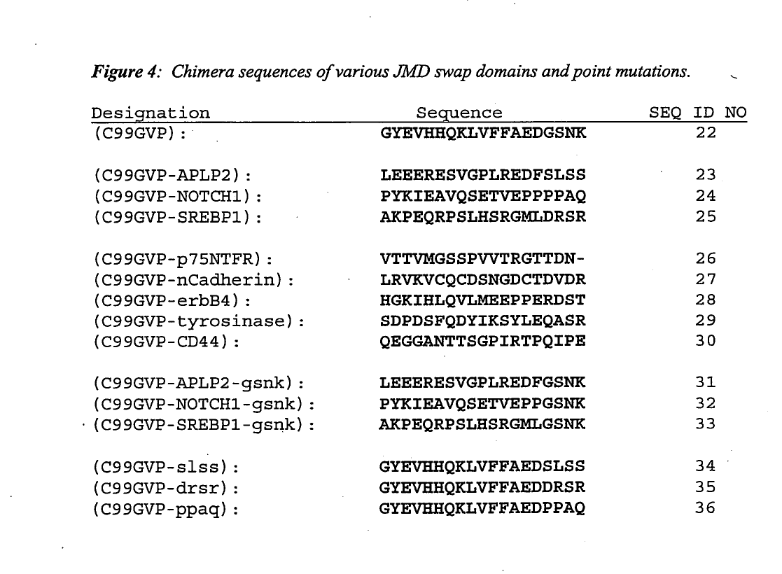

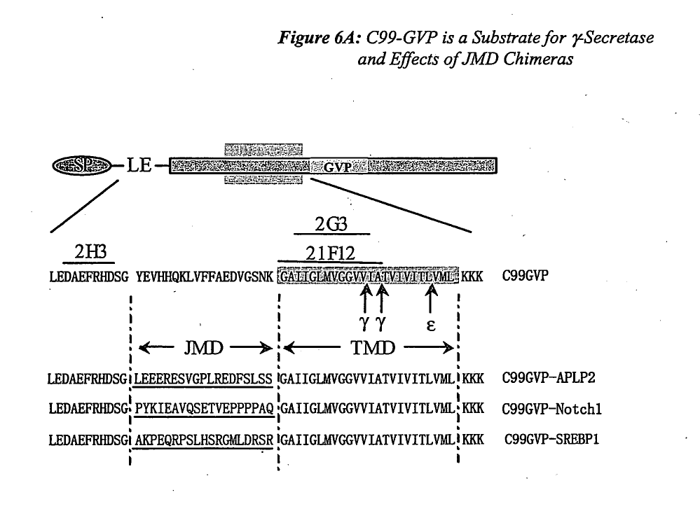

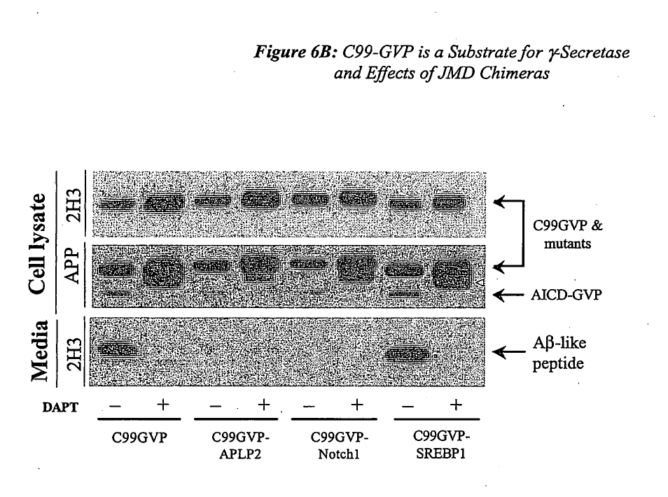

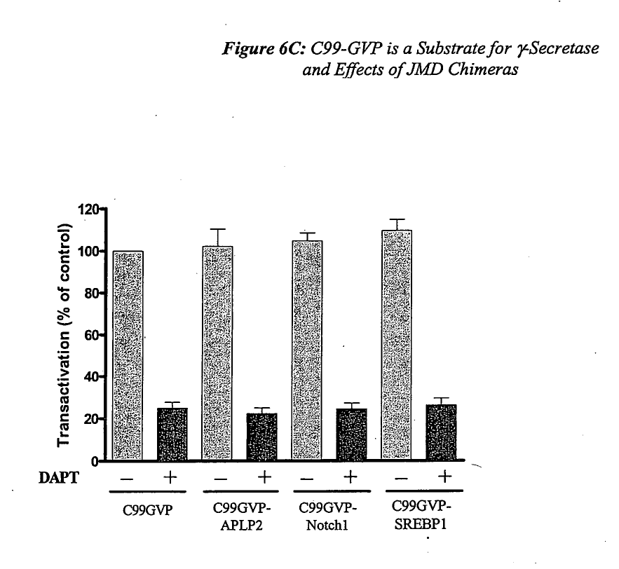

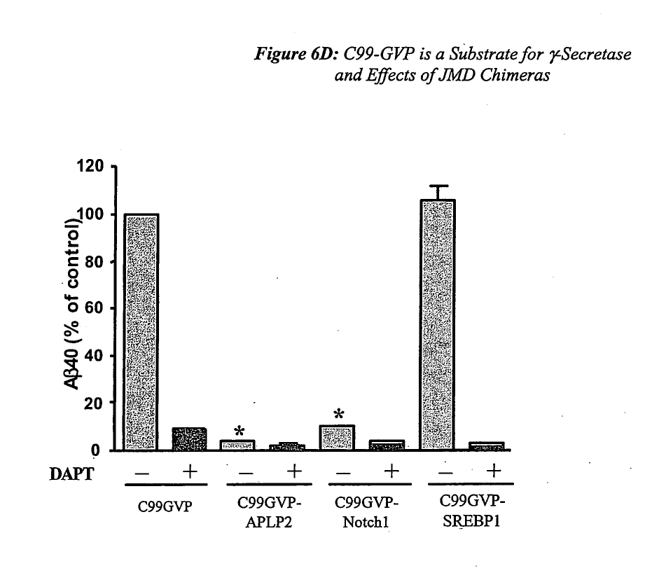

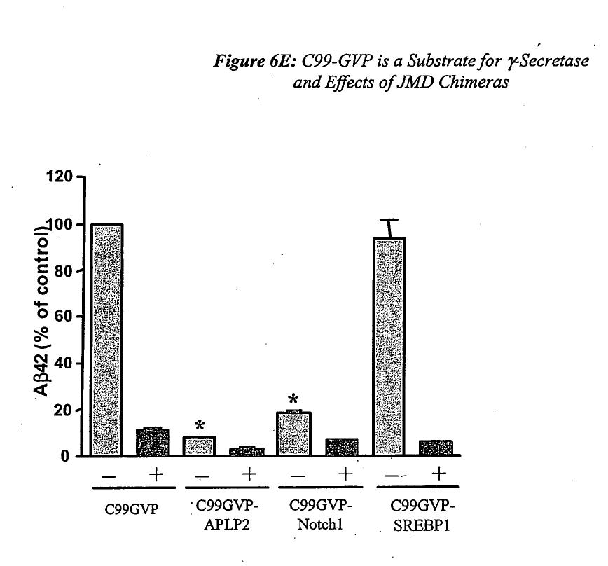

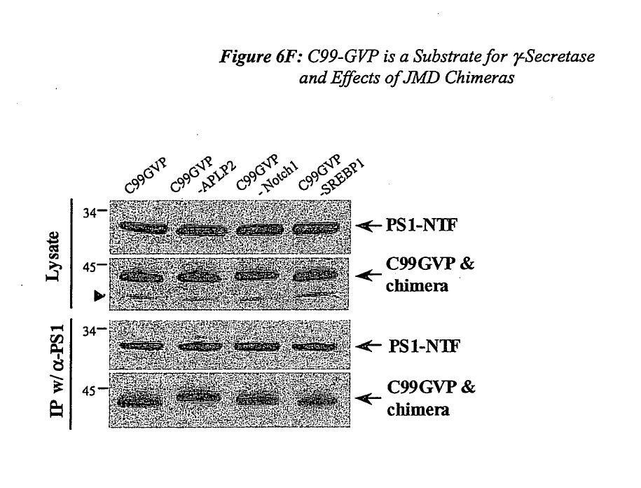

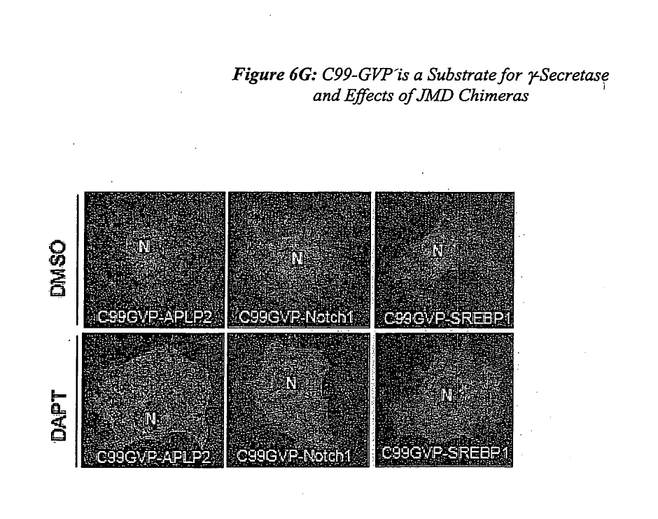

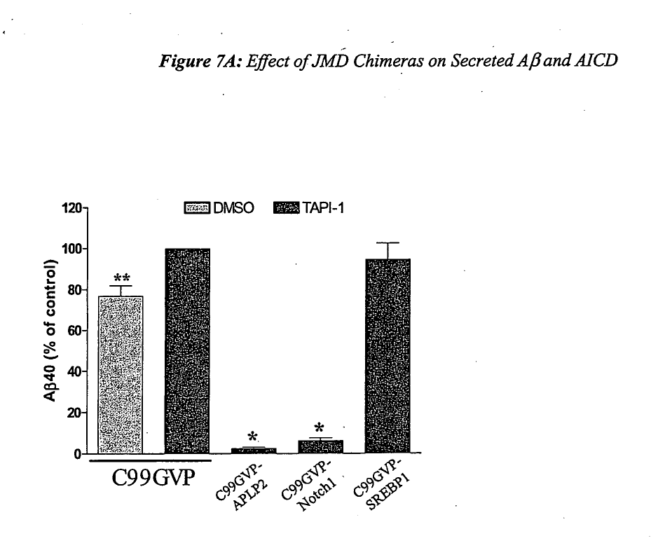

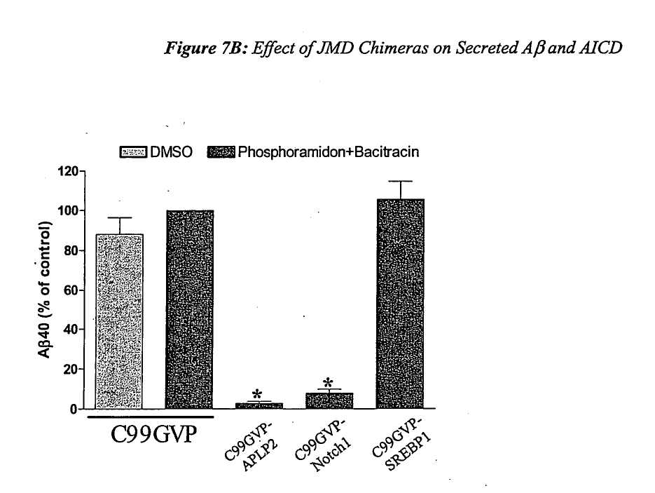

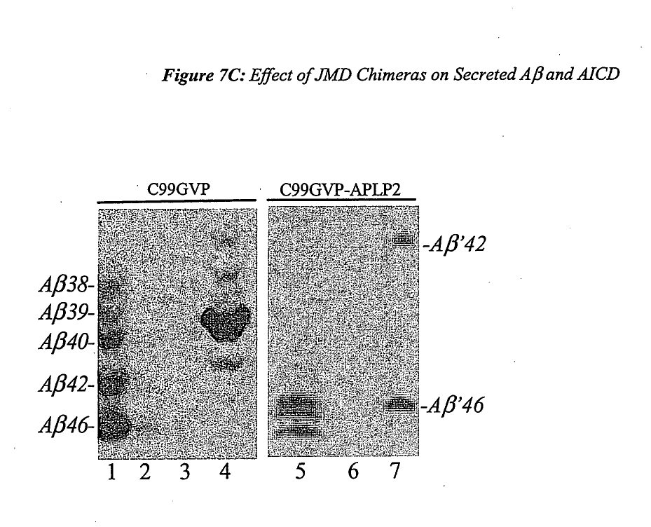

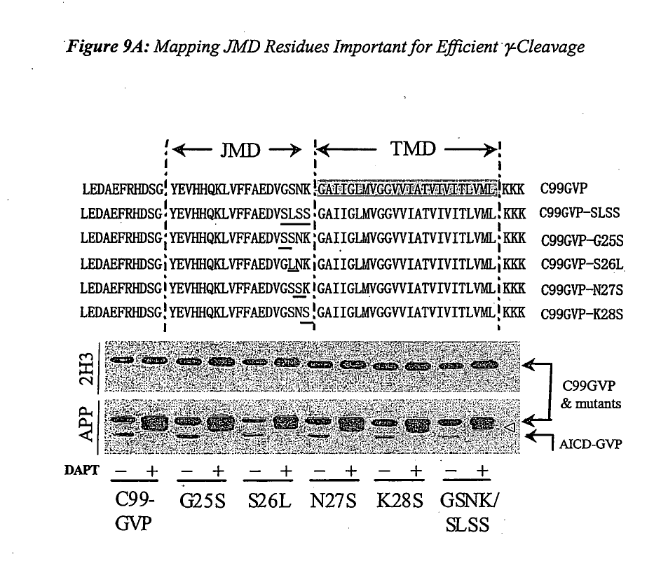

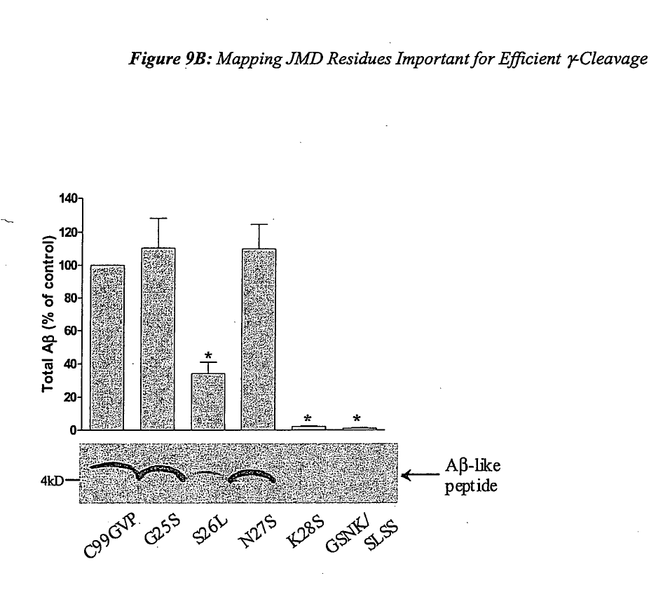

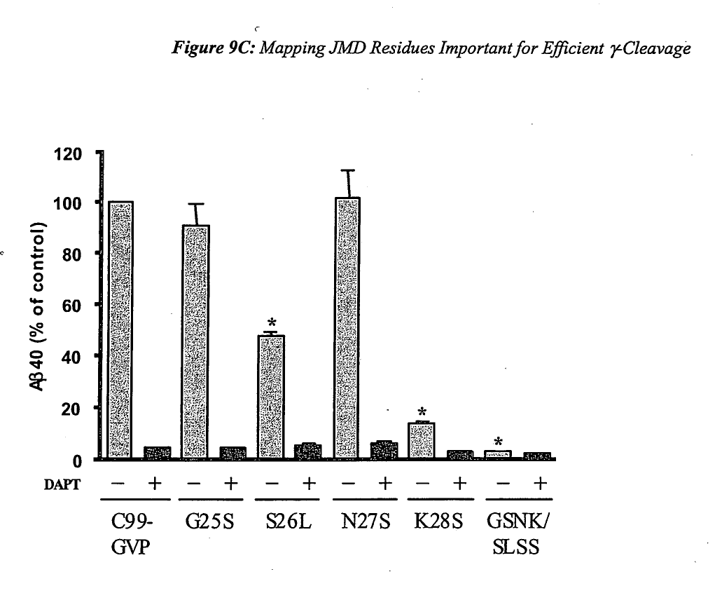

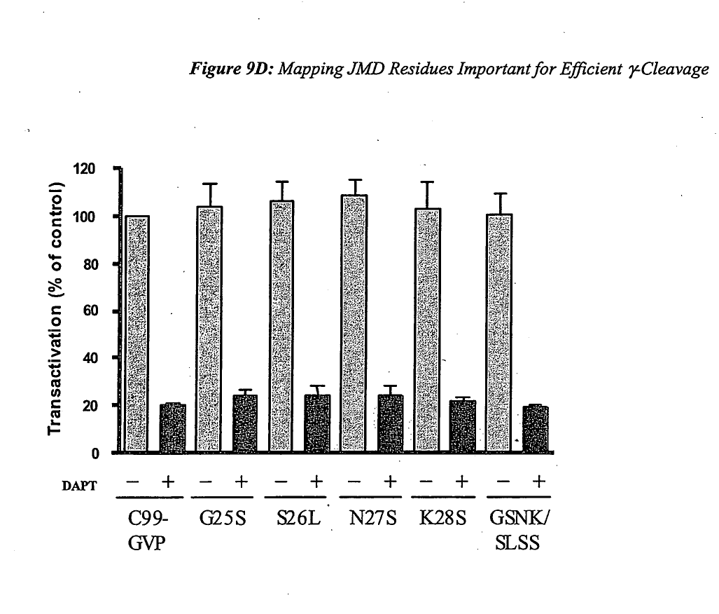

Plasmid Construction of JMD Chimeric Substrates: A pcDNA3.1-C99 plasmid similar to the previously described SPA4CT-LE construct (Dyrks, et al., FEBS Lett., 1992; 309: 20-24) was generated by standard PCR techniques. The APP signal peptide was fused to the N-terminus of the C99 fragment via a dipeptide leucine-glutamic acid (LE) linker. The strategy used to generate the pcDNA3.1-C99GVP construct was similar to a previously described method (Karlstrom, H., et al., J. Biol. Chem., 2002; 277: 6763-6766). (See, generally, FIG. 3). Briefly, an AscI site was introduced immediately 3′ of the nucleotides encoding the triple-lysine (K) membrane anchor of C99, where the GVP coding sequence was subsequently inserted in frame (to the C-terminal side of the lysine membrane anchor sequence of C99). To make a series of juxtamembrane gamma substrate chimeras, nucleotides encoding a 19-residue luminal juxtamembrane domain in C99GVP (corresponding to amino acids 606-625 in APP₆₉₅) was replaced by nucleotide sequences encoding for the corresponding regions from human APLP-2 (amino acids 674-693), Notch1 (amino acids 1716-1734) or SREBP1 (amino acids 6469-6487), generating the constructs C99GVP-APLP2, C99GVP-Notch1 and C99GVP-SREBP1, respectively. All three chimeras were constructed by using a two-stage PCR method with two pairs of overlapping primers (for list of primers, see Table I). These chimeric substrates were characterized to assess activity as gamma secretase substrates and subsequent production and secretion of Aβ and AICD (FIGS. 6-7). Additional domain swap chimeras retaining the pre-TMD GSNK motif of APP, designated as C99GVP-APLP2* or C99GVP-APLP2-GSNK, C99GVP-Notch1* or C99GVP-Notch-GSNK, and C99GVP-SREBP1* or C99GVP-SREBP1-GSNK, were generated in a similar fashion with a different set of primers. The C99GVP-SLSS quadruple JMD chimera was also constructed with the same PCR method. Point mutations within the luminal juxtamembrane domain (i.e., C99GVP-G25S, S26L, N27S and K28S; FIGS. 2 and 9A-D) were generated using QuikChange (Stratagene) site-directed mutagenesis kit according to the manufacturer's instructions. All cDNAs were verified by sequencing. The Aβ and Aβ-like peptides generated from C99GVP and the various chimeric substrates were numbered with reference to the first N-terminal residue (Asp-1) of the Aβ peptide.

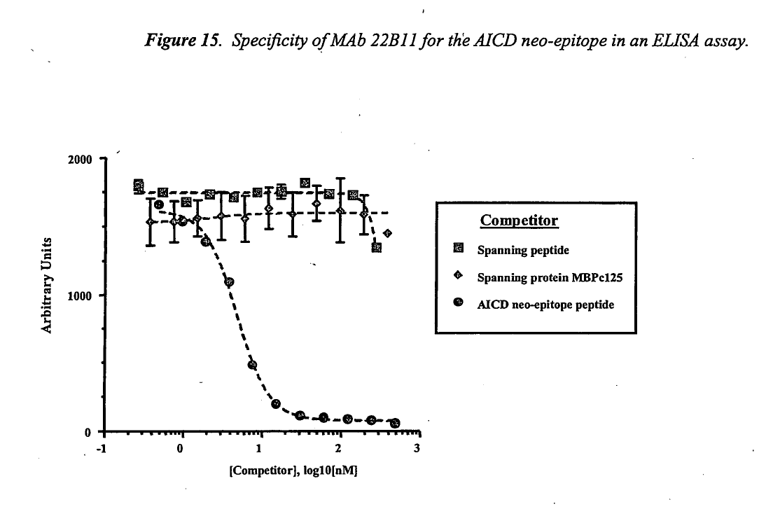

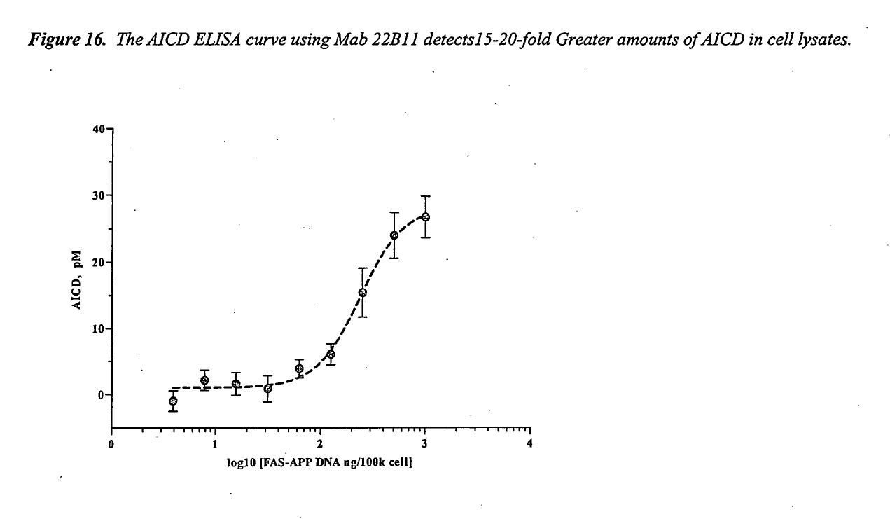

Antibodies: Polyclonal antibody against the last C-terminal 20 amino acids of APP and monoclonal anti-Flag were purchased from a commercial source (Sigma Cat. #A8717 and F1804) and used at 1:20,000 and 1:2,000 dilutions for Western blots, respectively. Monoclonal antibodies to VP16 (Santa Cruz Biotechnology Cat. #sc-I 728, Santa Cruz, Calif.) were used at 1:500 dilution for Western blots. Monoclonal antibodies, 2H3 (specific to Aβ4-7), 2G3 (specific to Aβ33-40) and 21F12 (specific to Aβ 30-42) were produced in house, as described previously (see, e.g., Johnson-Wood, K., et al., Proc. Natl. Acad. Sci. USA, 1997; 94: 1550-1555). Polyclonal and monoclonal [22B11] AICD neo-epitope antibodies were raised against the peptide VMLKKKC (SEQ ID NO:39). Characterization of the monoclonal [22B11] antibody by ELISA demonstrates that the antibody binds to the antigenic peptide in a dose-dependent manner. The [22B11] antibody does not cross-react with a peptide that contains the antigenic peptide along with the intact APP ε-cleavage site (TVIVITLVML KKKQTYTS, SEQ ID NO:91). The intact ε-cleavage site peptide (i.e. spanning the cleavage site and lacking the neo-epitope derived by cleavage) does not interfere with the binding between the [22B11] antibody and the antigenic peptide. (FIGS. 15 and 16).

Cell Culture and Transient Transfection: Human embryonic kidney 293 (HEK 293) cells (ATCC) were grown in Dulbecco's Modified Eagle Medium with High Glucose (DMEM, obtained from Gibco/Invitrogen, Cat #11960) supplemented with 10% fetal bovine serum (Hyclone, SV 30014.03) and 50 units/ml penicillin and streptomycin (37° C., 5% CO₂). Cells were used at less than passage number 30. At the time of seeding, viability of cells was greater than 95% as determined using a Vi-Cell Analyzer (Beckman-Coulter). Confluence of cells on plates was kept at greater than 95% during all phases of the experiment as determined with a standard tissue culture inverted microscope. All transfections were carried out on 5×10⁶ cells in 6-well tissue culture plates (Costar). The following 3 plasmids: pG5E1B-luc, 200 ng (gift from R. Maurer, OHSU); pCMV-β-gal, 100 ng (gift from R. Maurer, OHSU); C99GVP or the various chimeric constructs, 200-400 ng were added together to each well. FuGENE6 reagent (Roche Cat. #11-814443001) was used according to manufacturer's protocol for the transient transfection of adherent cells. Transfected cells were reseeded onto 12 well (2×10⁶ cells) and/or 96-well (5×10⁴ cells) plates (Costar) 16 h post-transfection; fresh media was added either with or without gamma secretase inhibitors. The cells and conditioned media were harvested 48 h post-transfection for analysis.

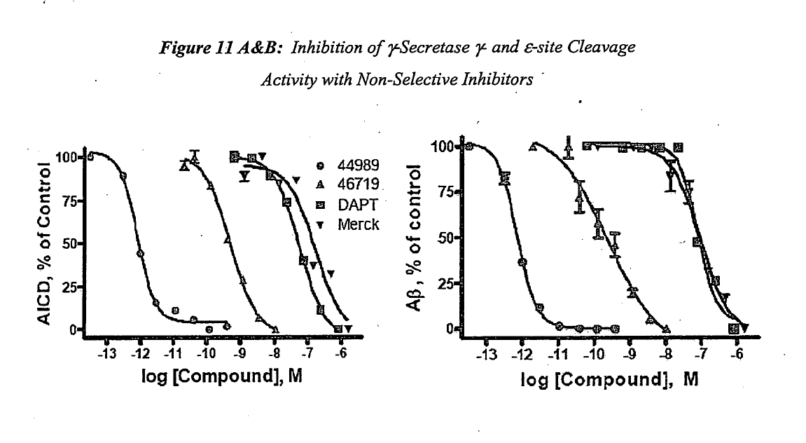

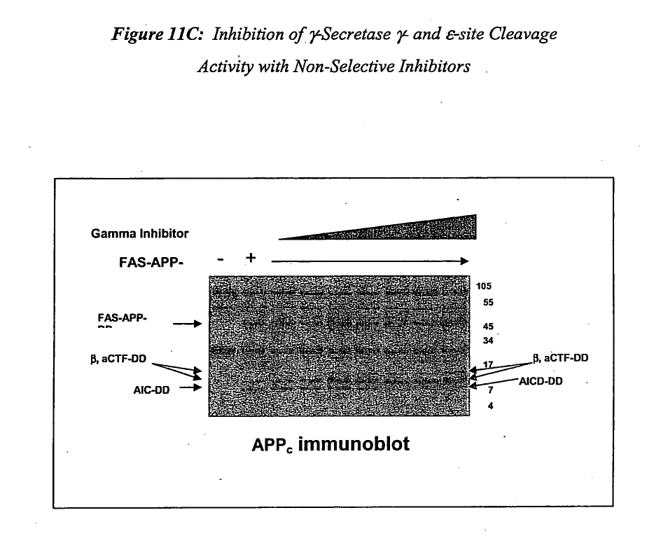

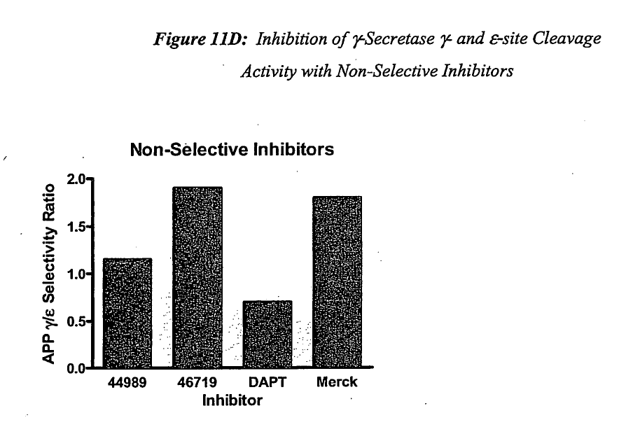

Inhibitor Treatment of Transfected HEK Cell: The transition state analogue gamma secretase inhibitor-L685,458 (Sigma) and the peptidomimetic inhibitor-DAPT (Dovey, H. F., et al., J Neurochem., 2001; 76:173-181) were dissolved in DMSO to make 20 mM stocks. Similarly, a number of Elan's series of sulfonamide inhibitors were prepared and used as described herein (see, also FIGS. 11-14). Inhibitors were added to cell cultures (e.g., HEK) at the indicated final concentration, and the treated cells were harvested 48 h post-transfection. The metallo-proteinase inhibitor TAPI-1 (Calbiochem) was used at a final concentration of 40 μM. The Aβ-degrading enzyme inhibitors, Bacitracin (Calbiochem) and phosphoramidon (Calbiochem) were used at final concentration of 1 mg/ml and 40 μM, respectively. All inhibitor experiments were performed in triplicate and repeated at least three times.

Western Blot Detection of the Substrates and AICD: Forty-eight hours after transient transfection, HEK cells grown in 12 or 6-well tissue culture plates were washed with cold TBS and homogenized in 1 ml of lysis buffer (0.1% SDS, 0.5% Deoxycholate and 1% NP-40 in TBS) with a protease inhibitor cocktail (SigmaAldrich Cat. #P8340). All samples were solubilized at 4° C. for 1 h and cleared by centrifugation at 14,000×g for 30 min. Aliquots of the supernatants were boiled for 5 min in Laemmli sample buffer and resolved on 10-20% Tris-Tricine SDS-PAGE (Invitrogen). The gels were then Western blotted with appropriate antibodies and visualized with Supersignal West Pico chemiluminescent substrate (Pierce Cat. #34080). All experiments were repeated at least three times.

Immunostaining: Wild type or transiently transfected COS-7 cells (ATCC) were fixed at room temperature with 2% paraformaldehyde in PBS for 20 min and subsequently permeabilized with 0.2% Triton X-100 in PBS for 10 min. C99GVP and mutant substrates as well as AICD-GVP were detected by incubating the samples sequentially with polyclonal anti-VP16 for 2 h and Rhodamine-conjugated donkey anti-goat secondary antibody (Jackson Laboratory Cat. #705-165-003) for 1 h. All staining was visualized on a Bio-Rad MRC 1024ES confocal microscope (Bio-Rad) and captured with a coupled CCD camera.

Luciferase Reporter Gene Assay: Luciferase reporter assays were carried out 48 hr post-transfection. Cells seeded on 96-well plates (BD Biosciences) were washed once with PBS and harvested in 20 μl of reporter lysis buffer (Promega) per well. After adding 100 μl of luminescent substrate (Promega), the luciferase activity was measured with a MicroLumatPlus microplate luminometer (Berthold Technologies). The β-galactosidase activity was measured similarly, using a luminescent β-galactosidase substrate (BD Biosciences). As a control for transfection efficiency and general effect on transcription, the luciferase activity was normalized by measuring β-galactosidase activity on a duplicate plate. All measurements were done in triplicate and repeated at least three times.

Immunoprecipitation (IP) and Western Blot Detection of Aβ: Total Aβ peptides in conditioned medium or cell lysate were immuno-precipitated at 4° C. overnight with 4 μg of the 2H3 antibody, followed by incubation with 50 μl of a 50% protein G-Sepharose (GE Healthcare) slurry for 1 hr and three washes in the same lysis buffer as described above in the Western Blot discussion. Proteins were eluted from the solid-phase immunoprecipitates in Laemmli sample buffer by heating at 70° C. for 5 min and resolved on 10-20% Tris-Tricine SDS-PAGE or the modified Tris-Tricine/8M urea gels (Qi-Takahara, Y., et al., J Neurosci. (2005; 25, 436-445). After transferring onto nitrocellulose membranes (Invitrogen), the membranes were heated to 98° C. for 5 min in PBS, immunostained with the 2H3 antibody and visualized with Super-signal West Pico chemiluminescent substrate (Pierce). Each experiment was repeated at least three times.

22B11 Monoclonal Antibody Production Procedure: Conjugation of the Peptide: The immunogen for 22B11 was peptide (NH2)-VMLKKK-C* (obtained by custom peptide synthesis from Anaspec, San Jose, Calif.) coupled to Sheep anti Mouse IgG (Jackson ImmunoResearch), where (NH2)-VMLKKK is the neo-epitope generated by epsilon cleavage of the APP TMD and the Cys (C*) is an artificially added amino acid for facilitating the coupling of the peptide to the carrier. The peptide was coupled by the following method. 10 mgs. of Sheep anti Mouse IgG (Jackson Immunochemicals) were dialyzed overnight against 10 mM Borate buffer pH 8.5. The dialyzed antibody was then concentrated to 2 mL. 10 mgs sulfo-EMCS (Molecular Sciences) was dissolved in one mL deionized water. A 40 molar excess of sulfo-EMCS was added dropwise to the sheep anti mouse IgG and then stirred for ten minutes. The activated sheep anti mouse was then desalted over a Pierce 10 mL presto column equilibrated with 0.1 M PO₄ 5 mM EDTA pH 6.5. Antibody containing fractions were pooled and diluted to approximately 1 mg/mL using the A280 and 1.4 as the extinction co efficient. A 40 molar excess of peptide was dissolved in 20 mL of 10 mM PO4 pH 8.0. Each dissolved peptide was added to 10 mgs. of sheep anti mouse and rocked at room temperature for 4 hours. The conjugates were then concentrated to less than 10 mL and dialyzed against PBS with several changes for both buffer exchange and removal of excess peptide. Samples were then 0.22μ filtered to sterilize and aliquoted into 1 mg. fractions and frozen at −20° C. A BCA protein assay from Pierce was used to determine the concentration of the conjugate using a horse IgG standard curve. Conjugation was determined by a molecular weight shift of the coupled peptides above the activated sheep anti mouse.

### Immunization and Screening Protocol

Antibody 22B11 was produced by immunizing A/J mice (Jackson Laboratories) with (NH₂)-VMLKKKC (SEQ ID NO:39) coupled to Sheep anti-mouse (Jackson ImmunoResearch) via an artificial cysteine (C*) added to the native sequence at the C-terminus and the linking reagent sulfo-EMCS (Molecular Sciences). Animals were injected on day 0, 14, 28 and titered on day 35. The highest titer mouse was fused using a modification of Kohler and Milstein and the resulting positives screened for reactivity on the peptide VMLKKKC (SEQ ID NO:39) and lack of reactivity on peptides that spin the region, in particular TVIVITLVMLKKKQYTS (SEQ ID NO:91) or MBP-C125 (APP C125 fused to maltose-binding protein, where APP C125 is ADRGLTTRPG SGLTNIKTEE ISEVKMDAEF RHDSGYEVHH QKLVFFAEDV GSNKGAIIGL MVGGVVIATV IVITLVMLKK KQYTSIHHGV VEVDAAVTPE ERHLSKMQQN GYENPTYKFF EQMQN (SEQ ID NO:92).

Materials used for hybridoma fusions and propagation were Polyethylene Glycol 4000 (PEG4000) 50% w/v in 75 mM HEPES (obtained from Roche Cat #783 641); Dulbecco's Modified Eagle Medium with High Glucose without Glutamine (DME, obtained from Gibco/Invitrogen, Cat #11960); Fetal Bovine Serum (FBS, obtained from Hyclone, SV 30014.03); 1M HEPES (obtained from Gibco, Cat #15630); 10 mM Hypoxanthine (from Sigma) prepared in the Elan Media Facility; 0.17 M NH₄Cl (from Sigma Tissue Culture Grade Reagents) prepared in the Elan Media Facility; SP2/0 AG14 cells (obtained from American Type Cell Collection) and recloned in the Elan Hybidoma Facility; Azaserine (Sigma Tissue Culture Grade Reagents, Cat #A1164-5MG); 50 mL Medium from confluent SP2/0 (collected in-house); Recombinant IL6 (obtained from Roche, Cat #1 444 581); 96 well tissue culture plates (obtained from Corning).

Fusion Protocol

The mouse is sacrificed by CO₂ narcosis followed by cervical dislocation and immersed in 70% ethanol for several minutes. The spleen is aseptically removed and placed in 5 mL of growth medium (DME high glucose without Glutamine, 20% FBS, 10⁴ M Hypoxanthine, 15 mM HEPES and 2 mM Glutamine).

The spleen is disassociated between the frosted ends of two sterile glass slides until a single cell suspension is obtained. The spleen cell suspension is then transferred to a 15 mL tube and pelleted by spinning at setting 4 (500×g) in an IEC clinical centrifuge for 5-10 minutes.

The cell pellet is resuspended in 7 mL of 0.17 M NH₄Cl at 4° C. and the large aggregates of debris are allowed to settle for 3-5 minutes. This is done to remove debris from the fusion and lyse the red blood cells. The single cell suspension is then pipetted off the debris pellet, transferred to a 50 mL tube, and growth medium is added to bring the volume to 50 mL, cells are counted and then pelleted as above.

SP2/0 Ag14 are in mid to late log phase. The SP2/0 cells are counted in the hemacytometer and enough SP2/0 cells are removed and spun down as above to give 1 SP2/0 to 4 spleen cells. The media from the sp2/0 are saved for selection media. The SP2/0 cells are resuspended in DME and the spleen cells are added. DME is added to a volume of 50 mL and the cell mixture is spun at setting 4 for 10 minutes.

The cell pellet is loosened by vortexing. One milliliter of PEG 4000 is added to the cell pellet while shaking. Cells are vortexed, and the PEG 4000 is allowed to be in contact with the cells for one to two minutes. Twenty-five milliliters of DME are added to the cell/PEG mixture, and incubated for one minute at room temperature. Twenty-five milliliters of growth medium are added, and incubated for one minute at room temperature. Cells are then spun at a setting of 4 for 10 minutes and resuspended in selection medium. (45 mL SP2/0 conditioned medium, 0.45 mL 2 mM Glutamine, 0.45 mL 10⁻² M Hypoxanthine, 200 ug azaserine, 2 mL FBS, 100 U/mL IL6, growth medium to bring the volume to 100 mL). The fusion is plated at 50 uL/well into fifteen 96 well tissue culture treated plates.

At day one post fusion 50 uL of growth medium is added to each well. At three to five days post fusion half of the medium is aspirated off and replaced with 100 uL of fresh growth medium. At day seven post fusion, hybridomas should be observed in >50% of the wells. At day 6-8 post fusion 100 μl medium is added. On day 10-12 post fusion, screening should take place.

### Example 1

### Generation of Antibodies to APP Intracellular Domain (AICD)

AICD Polyclonal Antibodies: Two AICD polyclonal antibodies were obtained through custom synthesis from a commercial source (Anaspec, San Jose, Calif.). The polyclonal antibodies both exhibit positive titers against the immunizing peptide VMLKKKC (SEQ ID NO:39). The antibodies were affinity purified against immobilized immunizing peptide. The specificity of the antibodies was confirmed through western blot and ELISA-based analysis. The affinity-purified AICD antisera recognized AICD, but not the chimeric α- and β-C-terminal fragments or holoprotein, demonstrating that the AICD antisera is specific for the cleaved AICD fragment.

AICD Monoclonal Antibody: A monoclonal antibody was synthesized against an N-terminal portion of the AICD amino acid sequence. The technique was performed as against the immunogenic peptide VMLKKKC (SEQ ID NO:39). See Kimberly, et al., Biochemistry 42(1):137-144 (2003). The resulting monoclonal antibody [22B11] shows specific binding to the N-terminal region of the AICD fragment generated by gamma secretase cleavage (discussed above; FIGS. 15 & 16)

### Example 2

### Aβ ELISA

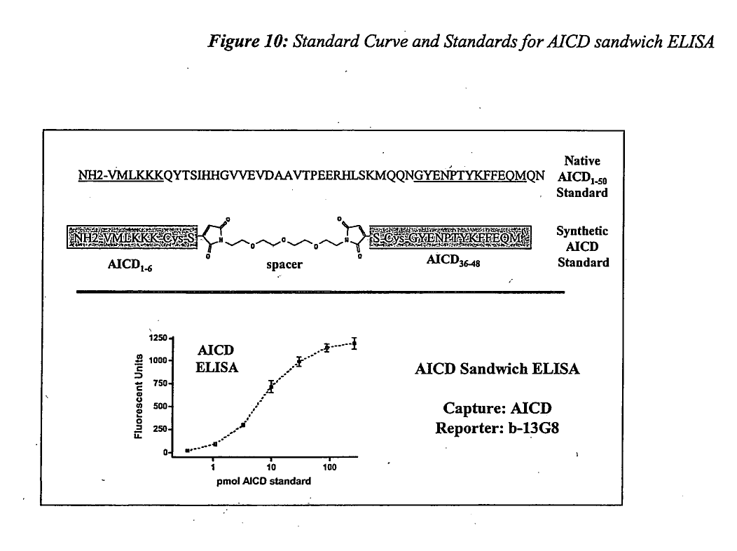

ELISAs used to quantify different Aβ species were performed using standard techniques as described above and in (Johnson-Wood, K., et al., Proc. Natl. Acad. Sci. U.S.A., 1997; 94: 1550-1555, incorporated by reference). The Aβ40 and Aβ42 peptides in the samples were captured onto 2G3 or 21F12 antibody coated plates, respectively, and detected with a biotinylated 2H3 antibody. The fluorescence signal generated from a streptavidin-alkaline phosphatase conjugate (Roche) was measured with a CytoFlour microplate reader (Applied Biosystems). Synthetic Aβ40 or Aβ42 peptides (Anaspec) were used to generate standard curves (FIG. 10). All measurements were done in triplicate.

### Example 3

### Quantitative Detection of ICD of APP (AICD)

An AICD sandwich ELISA was established based on capture of cell lysates with any of the AICD polyclonal or monoclonal antibodies discussed above, and reporting back with antibody directed at the extreme C-terminus of APP (e.g., 13G8, prepared in-house). Alternatively, luciferase-based reporter assays can be used to detect and quantify the presence of AICD and correlate those numbers to inhibitory potency of known or potential gamma secretase inhibitor compounds.

Synthetic AICD ELISA Standard. An AICD standard was synthesized by crosslinking AICD peptide and an APP C-terminal peptide (APP681-693; C-GYENP TYKFF EQM, SEQ ID NO:93) with 1,11-bis-maleimidotetraethylene-glycol (Pierce). The synthetic AICD standard was purified by reverse phase HPLC to >80% as determined by LC-mass spectrometry (data not shown). The total amount and concentration of the standard was determined based on it weight, purity and calculated molecular mass. The standard was validated based on further chemical characterization by mass spectrometry and reverse phase-HPLC, as well as its positive signal over background in the sandwich ELISA. (FIG. 10). Alternatively, full length, native sequence AICD peptide, (SEQ ID NO:41): VMLKKKQYTS IHHGVVEVDA AVTPEERHLS KMQQNGYENP TYKFFEQMQN, (Calbiochem Cat. #171545#) was used as a standard.

AICD ELISA using mAb against AICD: Standard curve. The monoclonal antibody [22B11], generated and purified as described above, was coated on a Thermolon 4HBX 96-well-plate, 100 μL at 10 μg/mL in coating buffer (0.23 g/L sodium monophosphate.2H₂O, 26.2 g/L sodium phosphate dibasic.7H₂O, sodium azide 1 g/L, 1 L q.s. pyrogen-free water), pH 8.0) at 4° C. for 48 h. After the incubation period the buffer solution was removed from the wells and discarded. To each well of was added 2004, of 0.25% blocking buffer (25 g/L crystalline Sucrose, 10.8 g/L Sodium phosphate dibasic-7H2O, 1 g/L Sodium Phosphate monoBasic-1H2O, 8.33 mL/L Human Serum Albumin 30% solution, Sodium Azide 0.5 g/L, 1 L q.s. pyrogen-free water, pH7.4), at 4° C. for overnight. After this incubation period, the blocking buffer was removed from the wells and discarded. The plates were placed in a chamber with a dessicant, under vacuum, overnight in order to allow the wells to dry completely. Anti-APP rabbit-polyclonal antibody, specific for the C-terminal region of APP (“Anti-APPcter”), was purchased from SigmaAldrich (Cat. #A8717) and was subsequently biotinylated using standard techniques. This modified antibody was used as a detecting antibody. Streptavidin-conjugated alkaline phosphatase (GE Healthcare formerly Amersham Cat. #RPN-1234) was used as the reporting system with in-house made Fluorescent Substrate A (31.2 g/L 2-amino-2-methyl-1-propanol, 30-33 mL/L 6N HCl, 0.03 g/L 4-methylumbelliferyl phosphate, q.s. IL High-quality water). Fluorescence Plate Reader (Cytofluor 4000 or Molecular Devices SpectraMax GeminiEM) was used to measure the signals in 96 well plates. APP-derived peptide CTF50 (≧95% purity by HPLC) was purchased from Calbiochem (Cat. #171545); having the sequence VMLKKKQYTS IHHGVVEVDA AVTPEERHLS KMQQNGYENP TYKFFEQMQN, (SEQ ID NO.41). This peptide was immunoprecipitated and captured on ELISA plates by mAb [22B11] and detected by Anti-APPcter rabbit-polyclonal antibody on western blots and in the ELISA assay, respectively. Control “spike and recovery” experiments using HEK293 cell lysates and cell lysates spiked with purified AICD peptides showed no shift in the standard curve, nor gave any appreciable background in the assay. Samples and Standards were diluted and bound to the plate in Casein diluent (8 g/L NaCl, 0.144 g/L Sodium Phosphate dibasic, 0.2 g/L Potassium Phosphate-monobasic, 0.2 g/L KCl, Casein 2.5 g/L, q.s. 1 L high-quality water, NaOH as needed to adjust to pH to 8.6).

Polyclonal antibody AICD ELISA. HEK 293 cells were grown under standard conditions to ˜90% confluence. Cells were harvested, counted, and subsequently plated onto PDL-coated 60 mm dishes at 2×10⁶ cells/dish in 5 mL media. The cells were allowed to settle onto the dishes for ˜4 hours. Transfection of various construct into cells was performed using standard techniques using Lipofectamine 2000™ (LF2K) (Invitrogen). Briefly, 2 μg plasmid DNA and 4 μL LF2K were diluted into separate 150 μL aliquots of Opti-MEM (Gibco), and allowed to stand for 10-15 minutes. The two aliquots were then mixed, and the DNA:Lipid complex allowed to form for about 20 minutes. The 300 μL DNA:Lipid complex was then added to the cells in 3 mL fresh media, and incubated overnight. In order to administer potential or known gamma secretase inhibitor compounds to cells, the transfected cells were harvested, replated into PDL-coated 24-well plates at 200,000 cells/well, and allowed to settle onto the plates for ˜4 hours. Cells were washed, and 500 μL fresh media added. The inhibitor compounds were added to the cells from a 10× concentration stock solution in DMSO, and allowed to incubate with the cells overnight (˜18 hours). After incubation the conditioned media (CM) was recovered from the cells, spun briefly, and saved for analysis using Aβ ELISA. The cells were washed once with PBS, followed by addition of 150 μL lysis buffer (PBS+0.5% NP40+Complete™ inhibitors (Roche)) to each well. Plates were incubated at 4° C. for 15 minutes, and the lysate recovered by centrifugation for 10 min. at 15,000×g. Supernatant was saved for protein determination and AICD ELISA. Typical protein yield is ˜0.45 mg/mL.

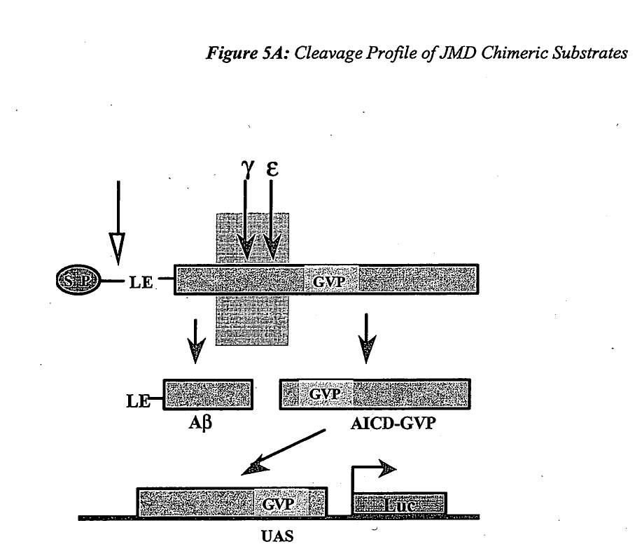

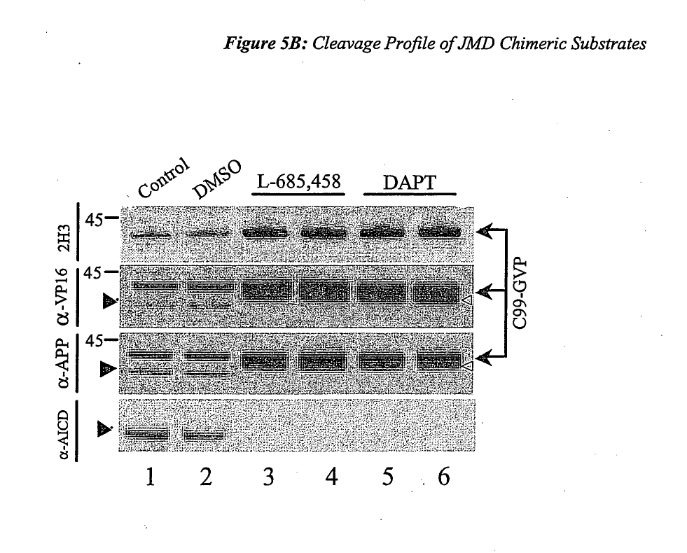

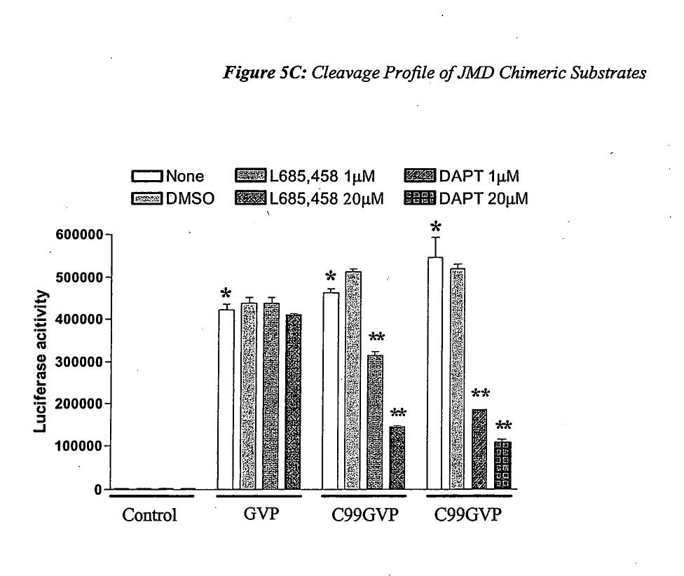

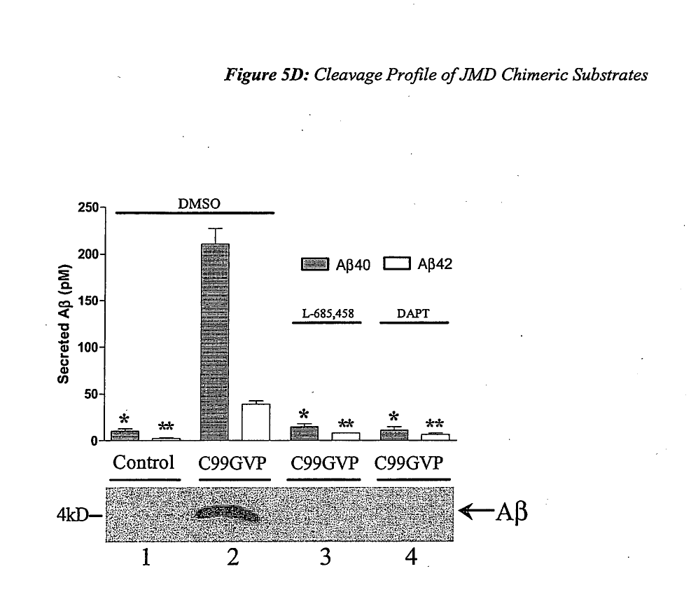

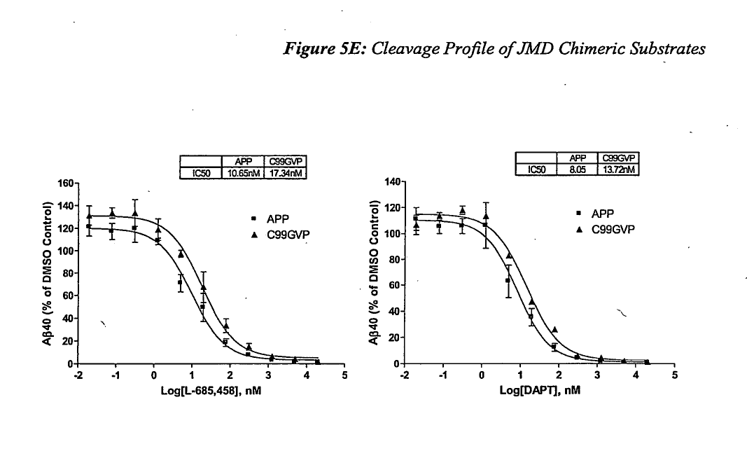

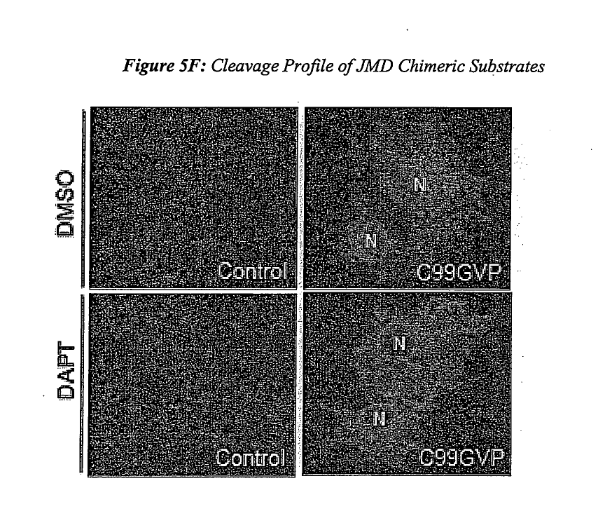

Luciferase Assay. After confirming AICD-GVP generation in HEK cells, its ability to transactivate a luciferase reporter gene that contains Gal4 response elements in the upstream activation sequence (UAS) was tested. No appreciable signal was detected from cells transfected with the reporter gene alone, whereas co-expressing an active form of GVP resulted in strong transactivation, thus confirming the specificity of this reporter assay. Robust signals, comparable to that of the GVP control, were also observed for cells cotransfected with C99GVP (FIG. 5C). Gamma secretase inhibitor treatment led to dose-dependent decrease of luciferase activity only in the C99GVP transfectant (FIG. 5C), indicating that C99GVP-induced reporter transactivation is gamma secretase-dependent. Some residual luciferase activity remained in the presence of excess gamma secretase inhibitors, even though identical treatment completely abolished AICD production as measured by Western blot (FIG. 5B). While this discrepancy may result from the extraordinary sensitivity and non-linear signal output of this assay (Karlstrom, H., et al., J. Biol. Chem., 1997; 277:6763-6766; Cao, X., and Sudhof, T. C., J. Biol. Chem., 2004; 279: 24601-611), it is likely not due to non-specific cleavage of the C99GVP cytoplasmic tail by other proteases.

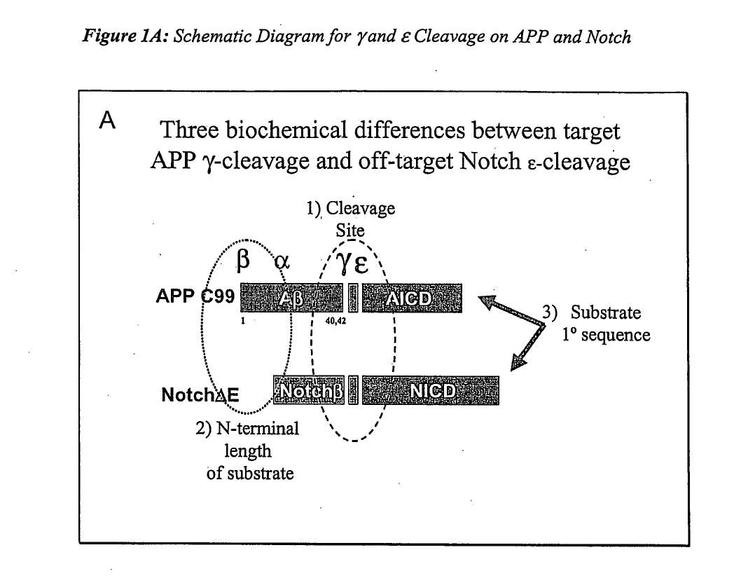

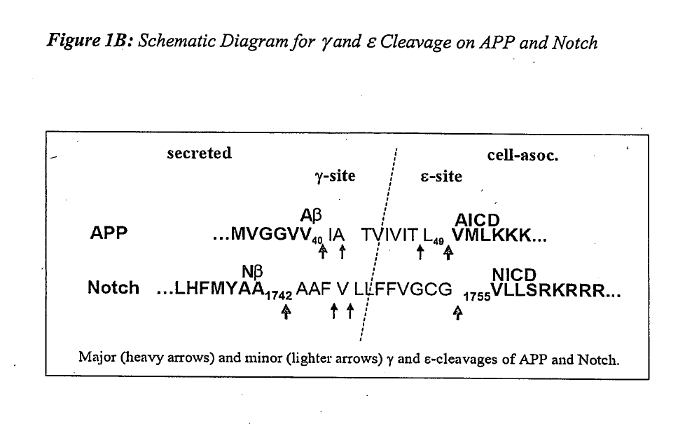

Next, Aβ generation was characterized from C99GVP. Wild type HEK cells and the mock-transfection control secreted little Aβ into the conditioned media (FIG. 5D, lane 1). In contrast, transient expression of C99GVP led to robust Aβ production, as measured by IP/Western blot (FIG. 5D) and ELISAs that detect Aβ 40 and Aβ42 species, respectively (FIG. 1D, top panel). Consistent with previous reports, Aβ 40 (210.8±19.2 μM) is the major secreted species, whereas Aβ42 (39.1±6.4 μM) only accounts for a small fraction (15.7±2.5%) of the total Aβ (FIG. 5D). γ-Secretase inhibitor treatment completely abolished Aβ secretion (FIG. 5D). Finally, we compared the Aβ-lowering potency of two inhibitors, using either C99GVP or the wild type APP as substrate. As determined by ELISA, the respective IC₅₀ values for the two substrates are essentially identical (FIG. 5E).

### Example 4

### Assay for Determining Gamma Secretase Substrate Specificity

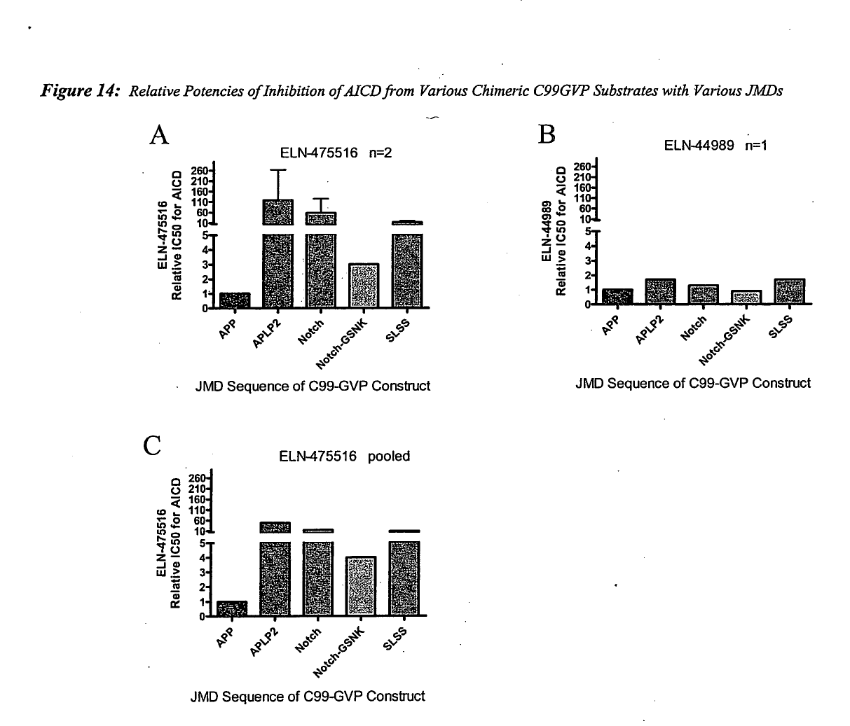

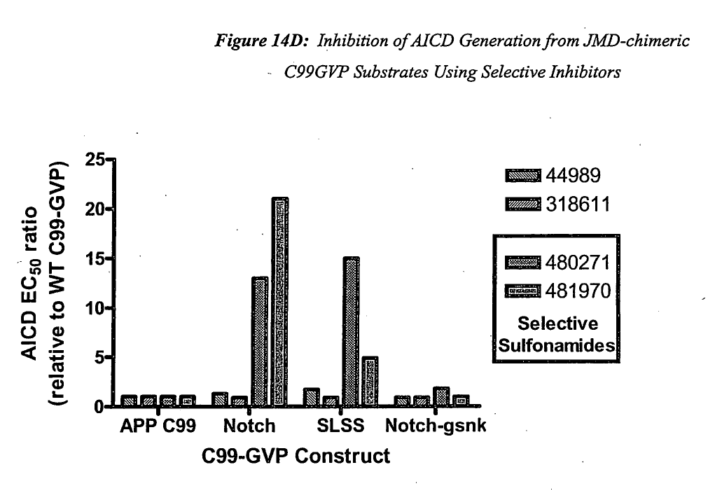

Several experiments were performed to test whether the juxtamembrane domain (JMD) of gamma secretase substrates might be involved in mediating or modulating the selectivity of certain types of gamma secretase inhibitor compounds. One experiment tested whether replacement of the JMD of APP-C99GVP with that from non-APP substrates such as Notch and APLP2 would right-shift the dose response curve for inhibition of AICD generation from these chimeric substrates relative to APP-C99-GVP with native JMD. The chimeric substrates were prepared generally as described above, and the gamma secretase activity assays performed using the above protocols (i.e., cells (HEK) were transfected and grown as described above. Gamma secretase activity in cells expressing C99G VP-Notch, C99GVP-APLP2, and C99GVP-APP in the presence of the inhibitor compounds was determined using ELISA and monoclonal antibody 22B11. The results in FIGS. 14A and 14B reveal that selective sulfonamide gamma inhibitors, 475516 and 477899 exhibited decreased AICD-inhibitory potency in cells transfected with C99GVP-Notch and C99GVP-APLP2 relative to C99GVP with native (APP) JMD. Non-selective compounds 44989 and 318611 failed to show and shift in potency with C99GVP-Notch and C99GVP-APLP2. Thus, the selectivity of compounds 475516 and 477899 for cleavage of the substrate was affected by the presence of a non-APP JMD.

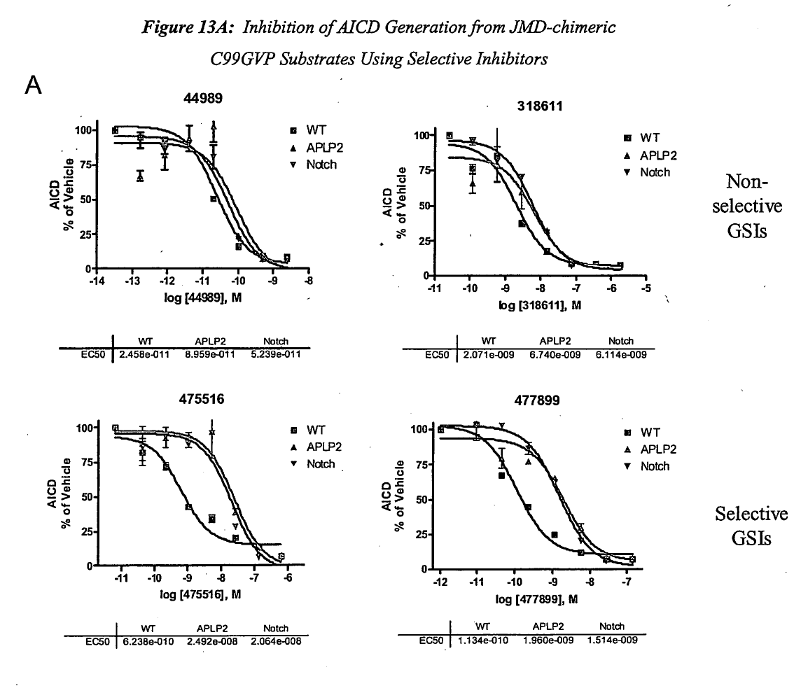

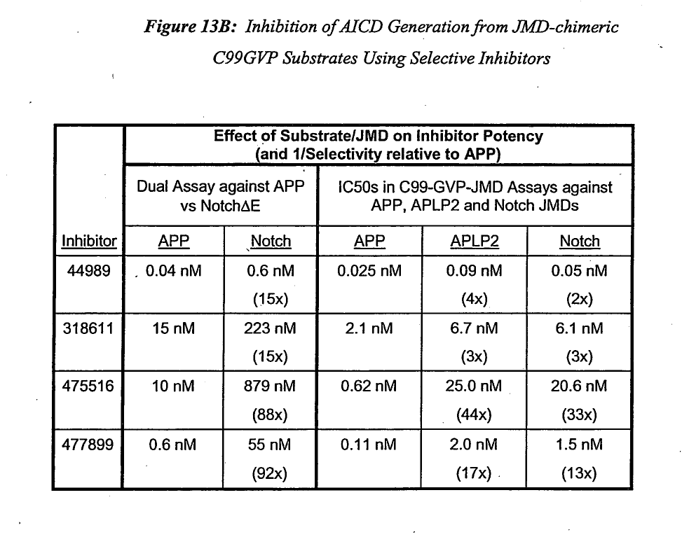

Another set of experiments were performed to repeat and extend the above findings. Briefly, cells (293) were transiently transfected with the indicated C99GVP constructs (native and chimeric) and then the concentration dependence of inhibition of AICD generation was analyzed with ELN-44989 and ELN-475516. The results from this study of the concentration-dependence of inhibition of AICD generation are summarized in FIG. 13. FIG. 13A shows EC₅₀ values (average EC₅₀ values from two replicate concentration-response experiments) for AICD inhibition with compounds 475516, 44989, 477899, and 318611 for the various constructs and were normalized to the IC₅₀ for C99-GVP with WT APP JMD (error bars indicate CVs based on replicate determinations of IC₅₀). The data shows an obvious right-shift in the potency of the selective inhibitors, 475516 and 477899 in cells expressing the chimeric C99-GVP Notch and APLP2 constructs, containing the non-APP JMD region.

These results demonstrate the right-shifted inhibition of AICD generation from C99GVP chimeras with APLP2 and Notch JMDs relative to APP JMD with selective inhibitors (FIG. 13A-B), but not with non-selective inhibitors. In another experiment, the right-shift of AICD inhibition observed with selective inhibitors and with Notch and APLP2 constructs appeared to be partially reversed using a C99GVP construct with Notch JMD construct retaining the APP ‘GSNK’ motif (C99GVP-NotchΔ4-GSNK (FIG. 14D). In a similar experiment substituting just the SLSS residues (from the JMD of APLP2) into the APP JMD of C99GVP (C99GVP-APPΔ4-SLSS) decreases the potency of inhibition of the selective compound 475516 to an EC₅₀ comparable to that of the full APLP2 JMD chimera (FIGS. 14A & 14C “SLSS”).

### Other C99GVP-JMD Chimeric Substrates

Using the general protocols described above, an additional series of C99GVP JMD chimeras were generated that included C99GVP-P75-NTR; C99GVP-N-Cadherin; C99GVP-ErbB4; C99GVP-SCNB2; and C99GVP-Tyrosinase. Constructs encoding these chimeras, as well as the C99GVP-Notch and C99GVP-APLP2 constructs, were transfected in HEK293 cells. Briefly, cells were plated on 10 cm dishes at 3.75×10⁶ cells/dish. After one day, the cells were transected with 12.5 μg per 10 cm dish of C99-GVP plasmid cDNA using the Fugene-6 reagent and 4:1 Fugene to cDNA ration (μL/μg). The following day, cells were plated on poly-D-lysine coated 96-well plates at 31,700 cells per well. On the next day, the cells were treated with compounds in media containing 0.4% DMSO(CO, 100 μL/96 well plate well. The cells were treated overnight and the plates were centrifuged. The cells were washed once with PBS containing Mg2+ and Ca2+ and were lysed in 25 mL of lysis buffer (1% TritonX100, 50 mM Tris, pH 7.5, 150 mM NaCl, 2 mM EDTA, plus complete protease inhibitor cocktail) for 1 hour at 4° C. on a rocker platform. The plates were centrifuged at 2100 rpm in a tabletop centrifuge (1000×g, 10 min,. at room temp). The supernatants (20 μL) were transferred onto a polypropylene storage plate and stored at −80° C. after freezing on dry ice. The supernatants were diluted on the storage plates with casein diluent (1:6 through 1:15) at the time of the ELISA. After mixing, 100 μL from each well was transferred onto a 22B11-coated ELISA plate using a 12-well pipette. A standard curve of 32-2000 pg/mL AICD was included on each plate. The plates were incubated at 4° C. overnight to allow binding of AICD. The following day the plates were washed 4× with TTBS (TBS with 0.05% Tween-20) incubated 1 hr in biotenylated Sigma anti-APP C-terminal antibody at a final concentration of 0.25 μg/mL in casein diluent. The plates were washed (as described above) and incubated for 1 hr at RT in Streptavidin-Alkaline Phosphatase (Roche) diluted 1:1000 in casein diluent. The plates were washed again and incubated for 30 min at room temperature in fluorescent substrate A. The plates were read using the SpectraMax GeminiEM plate reader and the data was analyzed using the SoftMax Pro software. For experiments performed in a 6-well plate, cells were plated at 0.625×10⁶ cells per well and transfected with the same method, using 2.1 μg cDNA per well. The cells in each 6-well plate were lysed in 1.25 mL of lysis buffer.

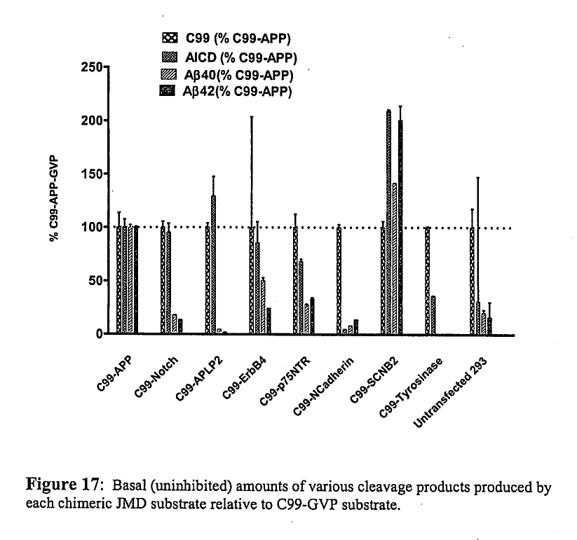

Using the AICD ELISA assays as described herein, the cell lysates were analyzed to measure the basal level effects that these JMD chimeras have on gamma secretase cleavage products in transfected cells. See (FIG. 17). The data presented in FIG. 17 is normalized to the amount of products for the C99-APP-GVP construct, thus all values are expressed as a percentage of C99-APP-GVP cleavage products.

### Effect of C99GVP-JMD Chimeric Substrates on Selective Gamma Secretase Inhibitors

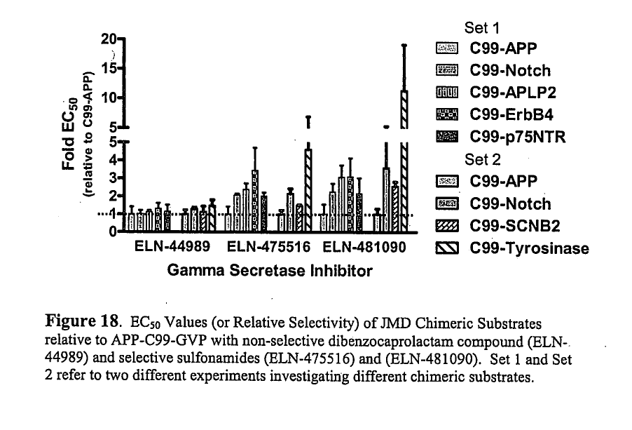

Assays were also conducted using the various chimeric C99GVP-JMD constructs (with JMD domains from different substrates) described above to determine whether the potency of certain sulfonamide-based selective gamma secretase inhibitor compounds would depend on the identity of the substrate JMD. A non-selective dibenzocaprolactam control compound, ELN-44989, and two selective sulfonamide inhibitor compounds, ELN-475516 and ELN-481090, were used to assess the effect that the various JMD constructs have on gamma secretase substrate specificity. The results for each compound are summarized in FIG. 18. The results are presented as “x-fold” EC50 values, relative to the value for the C99-APP-GVP construct. The non-selective compound ELN-44989 demonstrates that the change in substrates has little effect on the inhibitory potency of the compound on gamma secretase. However, the results for the selective sulfonamide compounds, ELN-475516 and ELN-481090, show that the different JMD C99-GVP substrate constructs have a significant effect on the EC₅₀ values of those compounds for gamma secretase, With the tyrosinase JMD construct having the largest effect. Thus, the sulfonamide compounds ELN-475516 and ELN-481090 display substrate selectivity among different substrate JMD constructs, with the greatest increase in ED₅₀ selectivity observed for the tyrosinase JMD construct.

### Example 5

### Role of GSNK Motif in Gamma Cleavage and Aβ Production

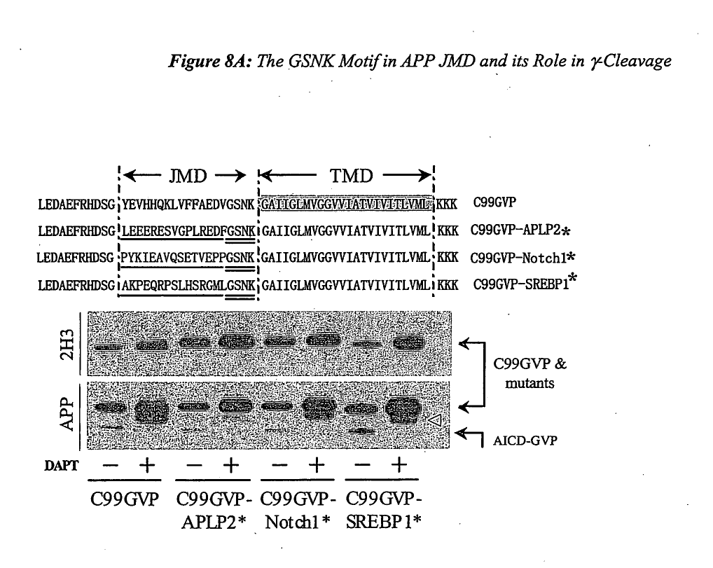

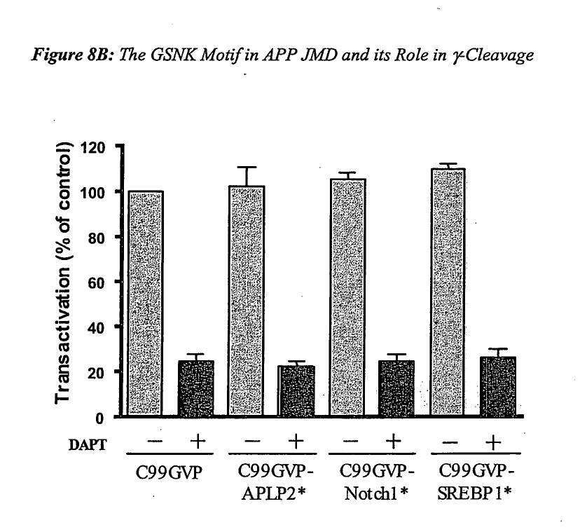

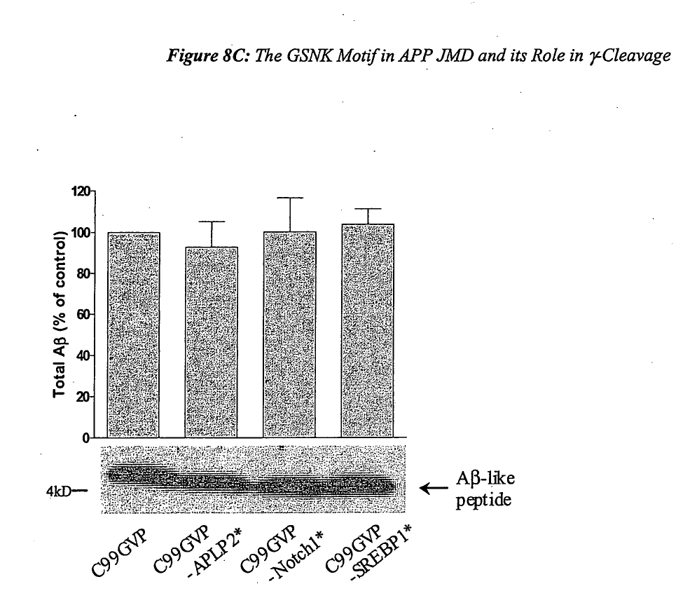

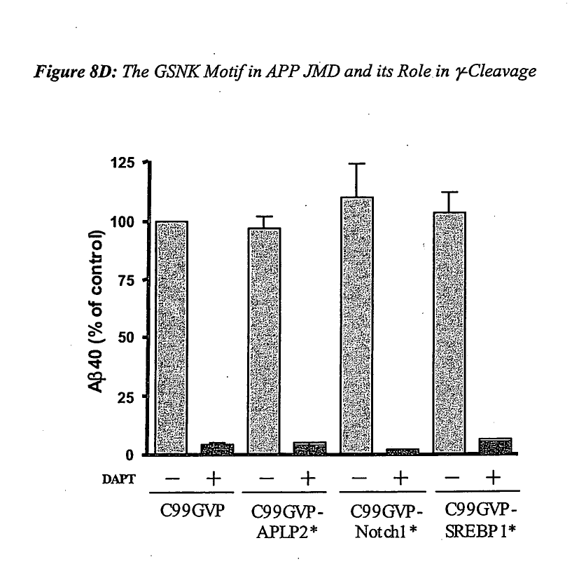

We have evaluated certain residues immediately preceding the TMD, partly because of their physical proximity to the intramembrane cleavage sites. In C99GVP as well as the full-length APP, the four amino acids N-terminal to the TMD are glycine-serine-asparagine-lysine (GSNK). The role of this four amino acid region of the JMD in A11 generation was investigated by retaining this tetrapeptide motif in a new set of chimeras, named C99GVP-APLP2-gsnk, C99GVP-Notch1-gsnk and C99GVP-SREBP1-gsnk, respectively, or alternatively identified by an asterisk (e.g., C99GVP-Notch1*) (See, e.g., FIG. 8A, top panel). The expression profile of these new chimeras was comparable to that of the C99GVP control (FIG. 8A, lower panels). In addition, little change was observed for AICD production (FIG. 8A, bottom panel) as well as AICD-GVP-mediated reporter transactivation (FIG. 8B). However, in marked contrast to their “native JMD swap predecessors,” the GSNK-containing C99GVP-APLP2* and C99GVP-Notch1* chimeras demonstrated robust Aβ production indistinguishable from the C99GVP control (FIGS. 8C and 8D). As expected, the C99GVP-SREBP1* chimera also maintained normal Aβ secretion (FIGS. 8C and 8D). These results clearly revealed a role for the GSNK motif in gamma cleavage and Aβ production. To further confirm this finding, we made another mutant, C99GVP-SLSS, in which the GSNK motif of C99GVP was substituted with a corresponding serine-leucine-serine-serine (SLSS) sequence from APLP2 (FIG. 9A, top panel). This mutation led to a marked reduction (˜97%) in secreted Aβ (FIG. 9B and FIG. 9C), but little change in AICD production (FIG. 9A, bottom panel) and reporter transactivation (FIG. 9D). These findings, along with the data obtained from the original juxtamembrane chimeras, demonstrate that even subtle alteration in the APP luminal juxtamembrane domain could lead to profound changes in gamma cleavage.

### Example 6

### Effects of Mutagenesis of Residues within GSNK Motif on Gamma Cleavage and Aβ Production

We also investigated the contribution of individual amino acids within the GSNK motif by mutating each of the four residues to the corresponding residues in APLP2 (FIG. 9A top panel). The point mutants, namely C99GVP-G25S, S26L, N27S and K28S, express comparably in HEK cells (FIG. 9A, lower panels). There was also little difference in their respective AICD production (FIG. 9A, lower panels) and signaling activity (FIG. 9D), again demonstrating equivalent ε-cleavage. However, substantial decrease in secreted Aβ was observed for both C99GVP-S26L and C99GVP-K28S mutants. The S26L mutation led to a 65.7±8.5% reduction in total AD and a 52.7±2.3% drop of Aβ40 (FIGS. 9B and 9C), whereas the K28S substitution resulted in an even more substantial (˜90%) decrease in both measurements (FIGS. 9B and 9C). In contrast, the other two mutations, G25S and N27S, showed no obvious effect on secreted Aβ (FIGS. 9B and 9C). Together, these data indicate that Lys-28 and Ser-26 are two preferred residues in the APP luminal juxtamembrane domain, and the substitution of which could selectively inhibit γ- but not ε-cleavage.

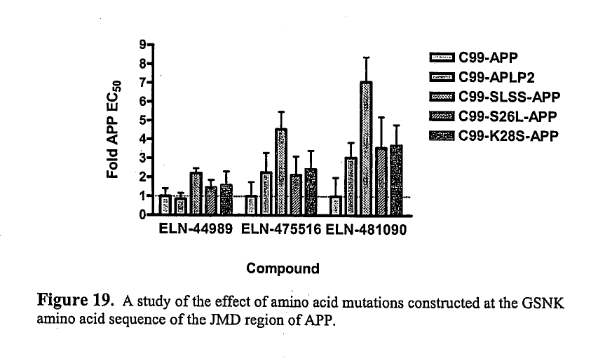

In a separate set of experiments, upon transient transfection into HEK-293 cells (see FIG. 19), the same four “APLP2” mutations introduced to the GSNK motif of APP (above) demonstrated an effect on potency of sulfonamide gamma secretase inhibitor compounds. A non-selective control compound, ELN-44989, and two selective inhibitor compounds, ELN-475516 and ELN-481090, were used to assess the effect of each point mutation on the substrate specificity of the inhibitor compounds. Using typical cell-based gamma secretase assay reaction conditions (e.g., as described herein), substitution of SLSS motif from APLP2 into JMD of APP (in place of naturally occurring GSNK of APP) produced a greater effect on inhibitor potencies of the selective compounds than observed with JMD of APLP2 alone. The potency of the non-selective compound 44989 was not affected (<2×) by substitution of SLSS into JMD of APP. Consistent with earlier observations described above, the two individual point mutation constructs lowered the potency of the selective inhibitors to an equivalent degree as observed with APLP2 JMD, (i.e. similar effect as the entire APP-APLP2 JMD construct). The S26L and K28S mutants increased the EC₅₀ value relative to C99-GVP-APP by about half as much as the construct which substitutes the four amino acid sequence, SLSS from APLP2 for the GSNK sequence of APP (FIG. 19).

### Example 7

### Selectivity of Cleavage at Gamma Compared to Epsilon

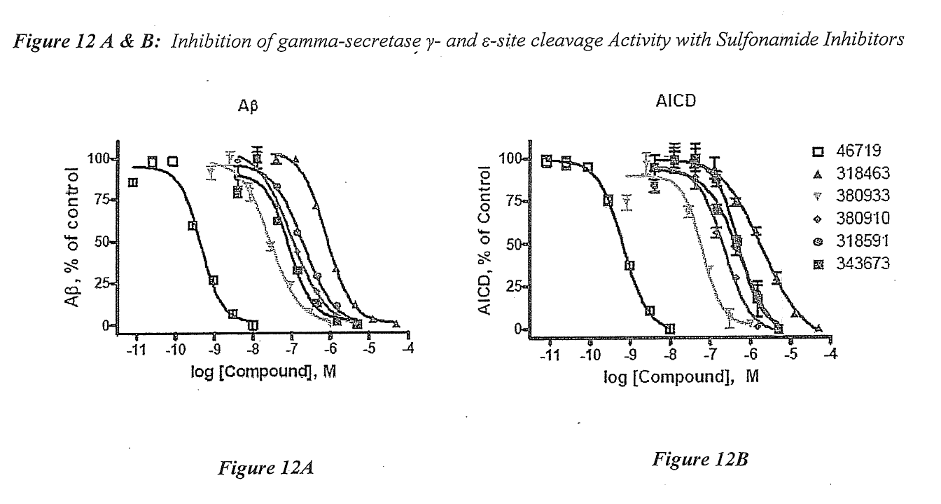

Treatment of Fas-APPsw-DD cells (Fas-APPsw-DD is a chimeric protein expressing Fas ectodomain fused to the C-terminal 125 amino acids of APP from Swedish FAD and that to the death domain residues 202-319 from FAS; Genbank M67454) with ‘non-selective’ gamma secretase inhibitors resulted in concurrent inhibition of both Aβ and AICD production (some data shown in FIGS. 5 & 6, and Table I; some data not shown). The term ‘non-selective’ in this instance refers to lack of selectivity for cell Aβ over Notch signaling (or GammaAPP over GammaNotch). Cellular Aβ and AICD inhibition curves with previously published, non-selective gamma secretase inhibitors and several of Elan's sulfonamide gamma secretase inhibitors are shown in FIGS. 11 and 12, respectively. FIG. 11 shows the Aβ and AICD IC₅₀s for DAPT, 44989, 46719 and Merck inhibitor compound L-685,458 and analysis of the γ/ε selectivity, calculated using the equation: γ/ε selectivity=IC₅₀ AICD/IC₅₀ Aβ. Aβ production was inhibited in the Fas-APPsw-DD transfected 293 cells with potencies generally in good agreement with historical data. In particular, Aβ production IC₅₀s ranged from 0.83-fold to 4.9-fold and averaged 3.0-fold higher in these experiments (from FIGS. 11-12) relative to historical data (excluding 44989 which paradoxically gave IC₅₀s 100-fold lower than historic data). A strength of this experimental system is that since the two ‘endpoints’ of this analysis (IC₅₀ values for γ and ε cleavages) are derived from a single cell (and substrate), the absolute potency and the absolute concentrations of the compounds is not as critical. The calculated γ/ε selectivity of the non-selective compounds (FIG. 11 and Table I) were 0.7, 1.1, 1.8 and 1.9 for DAPT, 44989, 46719 and the Merck compound, respectively. These values may not actually meaningfully differ from normality. For certain sulfonamides, the calculated γ/ε selectivity of (FIG. 14) ranged from 2.2 to 5.8, while for these compounds the cellular selectivity (EC₅₀ NotchSig/EC₅₀ Aβ) ranges from around 15-65 (FIG. 14). While 4 of the 5 sulfonamides exhibited APP γ/ε selectivities of 2.2-2.7, ELN-343673 has a γ/ε selectivity of 5.8. These sulfonamides exhibit 1.5 to 3.8-fold greater selectivity on average than ELN-46719 and other non-selective inhibitors. In other words, the data indicates that these sulfonamides do not seem to exhibit much selectivity for APP γ cleavage (relative to ε cleavages).

### Example 8

### Concurrent Measurement of Inhibitor Effects on App γ and ε Cleavage

The substrates and assays described above can be used to measure concurrently gamma secretase inhibitor effects on different cleavage sites on gamma secretase substrates (e.g., APP γ and ε cleavages). Such an assay is generally comprised of two parts, 1) inhibitor-treatment of cultured cells expressing a substrate of the invention, suitable for measurement of γ and ε cleavage products (e.g., Aβ and AICD) produced concurrently from the same cell culture, and 2) methods for quantitatively measuring the levels of both cleavage products. A gamma secretase substrate of the invention is able to generate two detectable gamma secretase cleavage products derived from different sites of cleavage on the substrate (generating a “A-beta like” peptide, and an ICD peptide). For detection of ICD a sandwich ELISA as described above is used. Routine ELISAs are used to quantify Aβ in conditioned medium. The utility of this technique lies in the fact that a selectivity value is derived from the ratio of two values derived from a single cellular experiment (e.g. simultaneous cells and compound-treatment for both assays). As a result, the selectivity value is expected to be less sensitive to inter-experiment variations and errors in compound dilution.

Assay Method using APP γ/ε. Cells: HEK 293 cells are grown under standard conditions to ˜90% confluence. Cells are harvested and counted, then plated onto PDL-coated 60 mm dishes at 2×10⁶ cells/dish in 5 mL media and allowed to settle onto the dishes for ˜4 hours. Cells are transfected using standard techniques, such as described above with Lipofectamine 2000™ (LF2K) (Invitrogen). The transfected cells are treated, inhibitor compound is added, and the cells are harvested all as described above for the ELISA assays.

It should be understood that the foregoing disclosure emphasizes certain specific embodiments of the invention and that all modifications or alternatives equivalent thereto are within the spirit and scope of the invention as set forth in the appended claims.

TABLE I
Primers Sequences used in JMD Chimera Constructs

| Primer ID | Primer Sequence | SEQ ID: |
|---|---|---|
| C99-GVP-F1 | cggctcgggc gctggaggat gcagaattcc gacatgactc | 52 |
| | aggatatgaa gttcatcatc | |
| C99-GVP-F2 | tggcactgct cctgctggcc gcctggacgg ctcgggcgct | 53 |
| | ggaggatgca g | |
| C99-GVP-F3 | caccaccatg ctgcccggtt tggcactgct cctgctggcc | 54 |
| | gcctggac | |
| APP-GVP-R | gcggccgcct agttctgcat ctgctcaaag aac | 55 |
| APLP-F | ccgtgggccc actgcgggag gacttcagtc tgagtagcgg | 56 |
| | tgcaatcatt ggactcatgg | |
| APLP-R | tcccgcagtg ggcccacgga ttcccgctct tcctcgagtc | 57 |
| | ctgagtcatg tcggaattc | |
| Notch-F | tgcagagtga gaccgtggag ccgcccccgc cggcgcaggg | 58 |
| | tgcaatcatt ggactcatgg | |
| Notch-R | tccacggtct cactctgcac ggcctcgatc ttgtagggtc | 59 |
| | ctgagtcatg tcggaattc | |
| SREBP-F | cgtctctgca cagccggggc atgctggacc gctcccgcgg | 60 |
| | tgcaatcatt ggactcatgg | |
| SREBP-R | ccccggctgt gcagagacgg ccgctgctct ggctttgctc | 61 |
| | ctgagtcatg tcggaattc | |
| p75NTR_R | cgggtcacca cgggctggga gctgcccatc actgtggtca | 62 |
| | ctcctgagtc atgtcggaat tct | |
| P75NTR_F | gcagctccca gcccgtggtg acccgaggca ccaccgacaa | 63 |
| | cggtgcaatc attggactca tggt | |
| nCad_R | cagtccccgt tggagtcaca ctggcaaacc ttcacgcgca | 64 |
| | gtcctgagtc atgtcggaat tctgc | |
| nCad_F | ttgccagtgt gactccaacg gggactgcac agatgtggac | 65 |
| | aggggtgcaa tcattggact catggt | |
| erbB4_R | taaagtggaa tggcccgtcc atgggtagta aatgcagtca | 66 |
| | tgtcctgagt catgtcggaa ttctgc | |
| erbB4_F | tacccatgga cgggccattc cactttacca caacatgcta | 67 |
| | gaggtgcaat cattggactc atggt | |
| Tyr_R | gttccaaata ggacttaatg tagtcttgaa aagagtctgg | 68 |
| | gtctgatcct gagtcatgtc ggaattctgc | |
| Tyr_F | cttttcaaga ctacattaag tcctatttgg aacaagcgag | 69 |
| | tcggggtgca atcattggac tcatggt | |
| SCN2B_R | agggggctct tccatgagga cctgcagatg gatcttgcca | 70 |
| | tgtcctgagt catgtcggaa ttctgc | |
| SCN2B_F | ctgcaggtcc tcatggaaga gccccctgag cgggactcca | 71 |
| | cgggtgcaat cattggactc atggt | |
| RHD/AAA-F | cgggcgctgg aggatgcaga attcgcagct gcctcaggat | 72 |
| | atgaagttca tcatc | |
| RHD/AAA-R | gatgatgaac ttcatatcct gaggcagctg cgaattctgc | 73 |
| | atcctccagc gcccg | |
| HHQK/AAQA-F | gttgctgctc aagcattggt gttctttgca gaagatgtgg | 74 |
| | gttc | |
| HHQK/AAQA-R | gaacaccaat gcttgagcag caacttcata tcctgagtca | 75 |
| | tgtcggaatt ctgcatcc | |
| ED/AA-F | ggtgttcttt gcagcagctg tgggttcaaa caaaggtgc | 76 |
| ED/AA-R | gcacctttgt ttgaacccac agctgctgca aagaacacc | 77 |
| APLP (GSNK)-F | ccgtgggccc actgcgggag gacttcggtt caaacaaagg | 78 |
| | tgcaatcatt ggactcatg | |
| Notch (GSNK)-F | tgcagagtga gaccgtggag ccgcccggtt caaacaaagg | 79 |
| | tgcaatcatt ggactcatgg | |
| SREBP (GSNK)-F | cgtctctgca cagccggggc atgctgggtt caaacaaagg | 80 |
| | tgcaatcatt ggactcatgg | |
| C99-SLSS-F | agaagatgtg agtctgagta gcggtgcaat cattggactc | 81 |
| | atggtgggc | |
| C99-SLSS-R | tgcaccgcta ctcagactca catcttctgc aaagaacacc | 82 |
| | aatttttgat gatgaac | |
| C99-G/S-R | ggtgttcttt gcagaagatg tgagttcaaa caaaggtgca | 83 |
| | atcattgg | |
| C99-G/S-R | ccaatgattg cacctttgtt tgaactcaca tcttctgcaa | 84 |
| | agaacacc | |
| C99-S/L-F | ggtgttcttt gcagaagatg tgggtttaaa caaaggtgca | 85 |
| | atcattggac | |
| C99-S/L-R | gtccaatgat tgcacctttg tttaaaccca catcttctgc | 86 |
| | aaagaacacc | |
| C99-N/S-F | ggtgttcttt gcagaagatg tgggttcaag caaaggtgca | 87 |
| | atcattggac tc | |
| C99-N/S-R | gagtccaatg attgcacctt tgcttgaacc cacatcttct | 88 |
| | gcaaagaaca cc | |
| C99-K/S-F | ggtgttcttt gcagaagatg tgggttcaaa ctcaggtgca | 89 |
| | atcattggac tcatgg | |
| C99-K/S-R | ccatgagtcc aatgattgca cctgagtttg aacccacatc | 90 |
| | ttctgcaaag aacacc | |

TABLE II
Amino Acid Sequences

| SEQ ID | Sequence | Description |
|---|---|---|
| 1 | DAEFRHDSGY EVHHQKLVFF AEDVGSNKGA | APP-C99 |
| | IIGLMVGGVV IATVIVITLV MLKKKQYTSI | |
| | HHGVVEVDAA VTPEERHLSK MQQNGYENPT | |
| | YKFFEQMQN | |
| 2 | KLLSSIEQAC DICRLKKLKC SKEKPKCAKC | GVP |
| | LKNNWECRYS PKTKRSPLTR AHLTEVESRL | |
| | ERLEQLFLLI FPREDLDMIL KMDSLQDIKA | |
| | LLTGLFVQDN VNKDAVTDRL ASVETDMPLT | |
| | LRQHRISATS SSEESSNKGQ RQLTVSGIPG | |
| | DLAPPTDVSL GDELHLDGED VAMAHADALD | |
| | DFDLDMLGDG DSPGPGFTPH DSAPYGALDM | |
| | ADFEFEQMFT DALGIDEYGG | |
| 3 | YEVHHQKLVF FAEDV | JMDΔA4-APP |
| 4 | LEEERESVGP LREDF | JMDΔ4-APLP2 |
| 5 | PYKIEAVQSE TVEPP | JMDΔ4-Notch |
| 6 | AKPEQRPSLH SRGML | JMDΔ4-SREBP |
| 7 | HDCIYYPWTG HSTLP | JMDΔ4-erbB4 |
| 8 | SDPDSFQDYI KSYLE | JMDΔ4-tyrosinase |
| 9 | VTTVMGSSPV VTRG | JMDΔ4-p75 NTFR |
| 10 | HGKIHLQVLM EEPPE | JMDΔ4-SCNB2 |
| 11 | LRVKVCQCDS NGDCT | JMDΔ4-n-Cadherin |
| 12 | QEGGANTTSG PIRTP | JMDΔ4-CD44 |
| 13 | GAIIGLMVGG VVIATVIVIT LVML | TMD: APP |
| 14 | DAEFRHDSG | A-beta |
| 15 | LEDAEFRHDS GYEVHHQKLV FFAEDVGSNK | JMD + TMD: C99-APP |
| | GAIIGLMVGG VVIATVIVIT LVML | |
| 16 | LE DAEFRHDSG LEEERESVGPLREDFSLSS | JMD + TMD: C99-APLP2 |
| | GAIIGLMVGG VVIATVIVIT LVML | |
| 17 | LE DAEFRHDSG PYKIEAVQSETVEPPPPAQ | JMD + TMD: C99-Notch |
| | GAIIGLMVGG VVIATVIVIT LVML | |
| 18 | LE DAEFRHDSG AKPEQRPSLHSRGMLDRSR | JMD + TMD: C99-SREBP |
| | GAIIGLMVGG VVIATVIVIT LVML | |
| 19 | LE DAEFRHDSG LEEERESVGPLREDFGSNK | JMD + TMD: C99-APLP2- |
| | GAIIGLMVGG VVIATVIVIT LVML | GSNK |
| 20 | LE DAEFRHDSG PYKIEAVQSETVEPPGSNK | JMD + TMD: C99-Notch- |
| | GAIIGLMVGG VVIATVIVIT LVML | GSNK |
| 21 | LE DAEFRHDSG AKPEQRPSLHSRGMLGSNK | JMD + TMD: C99-SREBP- |
| | GAIIGLMVGG VVIATVIVIT LVML | GSNK |
| 22 | GYEVHHQKLV FFAEDGSNK | JMD: APP |
| 23 | LEEERESVGP LREDFSLSS | JMD: APLP2 |
| 24 | PYKIEAVQSE TVEPPPPAQ | JMD: Notch |
| 25 | AKPEQRPSLH SRGMLDRSR | JMD: SREBP |
| 26 | VTTVMGSSPV VTRGTTDN | JMD: p75 NTFR |
| 27 | LRVKVCQCDS NGDCTDVDR | JMD: n-Cadherin |
| 28 | HGKIHLQVLM EEPPERDST | JMD: SCNB2 |
| 29 | SDPDSFQDYI KSYLEQASR | JMD: tyrosinase |
| 30 | QEGGANTTSG PIRTPQIPE | JMD: CD44 |
| 31 | LEEERESVGP LREDFGSNK | JMD: C99-APLP2-GSNK |
| 32 | PYKIEAVQSE TVEPPGSNK | JMD: C99-Notch-GSNK |
| 33 | AKPEQRPSLH SRGMLGSNK | JMD: C99-SREBP-GSNK |
| 34 | GYEVHHQKLV FFAEDSLSS | JMD: C99-APP-SLSS |
| | | (APLP2) |
| 35 | GYEVHHQKLV FFAEDDRSR | JMD: C99-APP-DRSR |
| | | (SREBP) |
| 36 | GYEVHHQKLV FFAEDPPAQ | JMD: C99-APP-PPAQ |
| | | (Notch) |
| 37 | LEDAEFRHDS G | A-beta + LE |
| 38 | VHHQKLVFFA EDVGSNKGAI IGLMVGGVVI | APP-C-terminal |
| | ATVIVITLVM LKKKQYTSIH HGVVEVDAAV | portion |
| | TPEERHLSKM QQNGYENPTY KFFEQMQN | |
| 39 | VMLKKKC | Immunogenic Peptide |
| | | for Ab production |
| | | (polyclonals as |
| | | well as mAb 22B11) |
| 40 | GYENPTYKFF EQM | |
| 41 | VMLKKKQYTS IHHGVVEVDA AVTPEERHLS | AICD(1-50) |
| | KMQQNGYENP TYKFFEQMQN | |
| 42 | LEDAEFRHDS GYEVHHQKLV FFAEDVSLSS | LE + JMD + TMD: C99- |
| | GAIIGLMVGG VVIATVIVIT LVML | APPA4-APLP2 |
| 43 | LEDAEFRHDS GYEVHHQKLV FFAEDVSSNK | LE + JMD + TMD: C99- |
| | GAIIGLMVGG VVIATVIVIT LVML | APP(G25S) |
| 44 | LEDAEFRHDS GYEVHHQKLV FFAEDVGLNK | LE + JMD + TMD: C99- |
| | GAIIGLMVGG VVIATVIVIT LVML | APP(S26L) |
| 45 | LEDAEFRHDS GYEVHHQKLV FFAEDVGSSK | LE + JMD + TMD: C99- |
| | GAIIGLMVGG VVIATVIVIT LVML | APP(N27S) |
| 46 | LEDAEFRHDS GYEVHHQKLV FFAEDVGSNS | LE + JMD + TMD: C99- |
| | GAIIGLMVGG VVIATVIVIT LVML | APP(K28S) |
| 47 | DAEFRHDSGY EVHHQKLVFF AEDVGSNKGA | C99-GVP (APP) |
| | IIGLMVGGVV IATVIVITLV MLKKKKLLSS | |
| | IEQACDICRL KKLKCSKEKP KCAKCLKNNW | |
| | ECRYSPKTKR SPLTRAHLTE VESRLERLEQ | |
| | LFLLIFPRED LDMILKMDSL QDIKALLTGL | |
| | FVQDNVNKDA VTDRLASVET DMPLTLRQHR | |
| | ISATSSSEES SNKGQRQLTV SGIPGDLAPP | |
| | TDVSLGDELH LDGEDVAMAH ADALDDFDLD | |
| | MLGDGDSPGP GFTPHDSAPY GALDMADFEF | |
| | EQMFTDALGI DEYGGQYTSI HHGVVEVDAA | |
| | VTPEERHLSK MQQNGYENPT YKFFEQMQN | |
| 48 | DAEFRHDSGL EEERESVGPL REDFSLSSGA | C99-GVP (APLP2) |
| | IIGLMVGGVV IATVIVITLV MLKKKKLLSS | |
| | IEQACDICRL KKLKCSKEKP KCAKCLKNNW | |
| | ECRYSPKTKR SPLTRAHLTE VESRLERLEQ | |
| | LFLLIFPRED LDMILKMDSL QDIKALLTGL | |
| | FVQDNVNKDA VTDRLASVET DMPLTLRQHR | |
| | ISATSSSEES SNKGQRQLTV SGIPGDLAPP | |
| | TDVSLGDELH LDGEDVAMAH ADALDDFDLD | |
| | MLGDGDSPGP GFTPHDSAPY GALDMADFEF | |
| | EQMFTDALGI DEYGGQYTSI HHGVVEVDAA | |
| | VTPEERHLSK MQQNGYENPT YKFFEQMQN | |
| 49 | DAEFRHDSGP YKIEAVQSET VEPPPPAQGA | C99-GVP (Notch) |
| | IIGLMVGGVV IATVIVITLV MLKKKKLLSS | |
| | IEQACDICRL KKLKCSKEKP KCAKCLKNNW | |
| | ECRYSPKTKR SPLTRAHLTE VESRLERLEQ | |
| | LFLLIFPRED LDMILKMDSL QDIKALLTGL | |
| | FVQDNVNKDA VTDRLASVET DMPLTLRQHR | |
| | ISATSSSEES SNKGQRQLTV SGIPGDLAPP | |
| | TDVSLGDELH LDGEDVAMAH ADALDDFDLD | |
| | MLGDGDSPGP GFTPHDSAPY GALDMADFEF | |
| | EQMFTDALG IDEYGGQYTSI HHGVVEVDAA | |
| | VTPEERHLSK MQQNGYENPT YKFFEQMQN | |
| 50 | DAEFRHDSGP YKIEAVQSET VEPPGSNKGA | C99-GVP |
| | IIGLMVGGVV IATVIVITLV MLKKKKLLSS | (Notch-GSNK) |
| | IEQACDICRL KKLKCSKEKP KCAKCLKNNW | |
| | ECRYSPKTKR SPLTRAHLTE VESRLERLEQ | |
| | LFLLIFPRED LDMILKMDSL QDIKALLTGL | |
| | FVQDNVNKDA VTDRLASVET DMPLTLRQHR | |
| | ISATSSSEES SNKGQRQLTV SGIPGDLAPP | |
| | TDVSLGDELH LDGEDVAMAH ADALDDFDLD | |
| | MLGDGDSPGP GFTPHDSAPY GALDMADFEF | |
| | EQMFTDALGI DEYGGQYTSI HHGVVEVDAA | |
| | VTPEERHLSK MQQNGYENPT YKFFEQMQN | |
| 51 | DAEFRHDSGY EVHHQKLVFF AEDVSLSSGA | C99-GVP |
| | IIGLMVGGVV IATVIVITLV MLKKKKLLSS | (APPA4-SLSS) |
| | IEQACDICRL KKLKCSKEKP KCAKCLKNNW | |
| | ECRYSPKTKR SPLTRAHLTE VESRLERLEQ | |
| | LFLLIFPRED LDMILKMDSL QDIKALLTGL | |
| | FVQDNVNKDA VTDRLASVET DMPLTLRQHR | |
| | ISATSSSEES SNKGQRQLTV SGIPGDLAPP | |
| | TDVSLGDELH LDGEDVAMAH ADALDDFDLD | |
| | MLGDGDSPGP GFTPHDSAPY GALDMADFEF | |
| | EQMFTDALGI DEYGGQYTSI HHGVVEVDAA | |
| | VTPEERHLSK MQQNGYENPT YKFFEQMQN | |
| 52-90 | Table I Primers | Nucleotide primers |
| 91 | TVIVITLVML KKKQTYTS (spanning peptide) | Spanning peptide |
| 92 | ADRGLTTRPG SGLTNIKTEE ISEVKMDAEF | APP-C-terminal 125 |
| | RHDSGYEVHH QKLVFFAEDV GSNKGAIIGL | fragment |
| | MVGGVVIATV IVITLVMLKK KQYTSIHHGV | |
| | VEVDAAVTPE ERHLSKMQQN GYENPTYKFF | |
| | EQMQN | |
| 93 | CGYENP TYKFF EQM | |
| 94 | QHAR | X1-X4 (erbB4) |
| 95 | QASR | X1-X4 (tyrosinase) |
| 96 | TTDN | X1-X4 (p75 NTFR) |
| 97 | RDST | X1-X4 (SCNB2) |
| 98 | DVDR | X1-X4 (n-Cadherin) |
| 99 | QIPE | X1-X4 (CD44) |
| 100 | PPAQ | X1-X4 (Notch) |
| 101 | DRSR | X1-X4 (SREBP) |
| 102 | SLSS | X1-X4 (APLP2) |
| 103 | GSNK | X1-X4 (APP) |

1-3. (canceled)

4. A method for determining whether a compound inhibits gamma secretase in a substrate specific manner, comprising:
(a) contacting a first gamma secretase substrate comprising a gamma secretase cleavage site with the compound and gamma secretase under conditions that allow for gamma secretase activity;
(b) separately contacting a second gamma secretase substrate comprising a gamma cleavage site with the compound and gamma secretase under conditions that allow for gamma secretase activity;
(c) determining the amount of gamma secretase activity at the gamma cleavage site of the first substrate and the second substrate;
(d) comparing the amounts of gamma secretase activity at the gamma cleavage site from step (a) with the amount of gamma secretase activity at the gamma cleavage site from step (b) and determining that the compound inhibits gamma secretase in a substrate specific manner when the amount of gamma secretase activity at the gamma cleavage site from step (a) is different from step (b),
wherein the second gamma secretase substrate comprises the formula:
[JMDΔC4]-X1-X2-X3-X4-[TMD] (Formula II);
wherein
[JMDΔC4] comprises the amino acid sequence of a juxtamembrane domain (JMD) sequence of a gamma secretase substrate, wherein the JMD lacks the four C-terminal peptides;
[TMD] comprises a transmembrane domain sequence of a gamma secretase substrate; and
X1, X2, X3, and X4 are independently selected from any amino acid.

5. The method of claim 4, wherein
X1 is selected from S, T, G, P, Q, R, V, L, N, P, A, K, E, I, F, H, W, and D;
X2 is any amino acid;
X3 is selected from S, N, D, P, E, R, T, F, I, K, L, V, G, W, H, and A; and
X4 is any amino acid.

6. The method of claim 4, wherein
X1 is selected from S, T, G, P, Q, R, V, L, N, P, A, K, E, I, F, H, W, and D;
X3 is selected from S, N, D, P, E, R, T, F, I, K, L, V, G, W, H, and A; and
X2 and X4 are selected from L, I, H, E, V, A, S, T, D, N, P, K, Q, and R.

7. The method of claim 4, wherein
X1 is selected from S, T, G, P, Q, R, and D;
X2 is any amino acid;
X3 is selected from S, N, D, P, and A; and
X4 is any amino acid.

8. The method of claim 4, wherein X1-X2-X3-X4 of the second gamma secretase substrate comprises GLNK, SLSS, GSNK, GSNS, PPAQ, SSNK, GSSK, QHAR, QASR, TTDN, RDST, DVDR, or QIPE.

9. The method of claim 4, wherein [TMD] of the second gamma secretase substrate comprises SEQ ID NO:13.

10. The method of claim 4, wherein [JMDΔC4] of the second gamma secretase substrate is selected from SEQ ID NOs: 3-5, or 7-12.

11. The method of claim 4, wherein the second gamma secretase substrate of Formula II comprises a sequence selected from the group consisting of:
(a) (C99GVP-APLP2):
(SEQ ID NO: 16)
LEDAEFRHDS GLEEERESVG PLREDFSLSS GAIIGLMVGG
VVIATVIVIT LVML;
(b) (C99GVP-NOTCH1):
(SEQ ID NO: 17)
LEDAEFRHDS GPYKIEAVQS ETVEPPPPAQ GAIIGLMVGG
VVIATVIVIT LVML;
(c) (C99GVP-SREBP1):
(SEQ ID NO: 18)
LEDAEFRHDS GAKPEQRPSL HSRGMLDRSR GAIIGLMVGG
VVIATVIVIT LVML;
(d) (C99APPA4-APLP2):
(SEQ ID NO: 42)
LEDAEFRHDS GYEVHHQKLV FFAEDVSLSS GAIIGLMVGG
VVIATVIVIT LVML;
(e) (C99-APP-(G25S):
(SEQ ID NO: 43)
LEDAEFRHDS GYEVHHQKLV FFAEDVS SNK GAIIGLMVGG
VVIATVIVIT LVML
(f) (C99-APP-(S26L):
(SEQ ID NO: 44)
LEDAEFRHDS GYEVHHQKLV FFAEDVGLNK GAIIGLMVGG
VVIATVIVIT LVML
(g) (C99-APP-(N27S):
(SEQ ID NO: 45)
LEDAEFRHDS GYEVHHQKLV FFAEDVGSSK GAIIGLMVGG
VVIATVIVIT LVML
(h) (C99-APP-(K28S):
(SEQ ID NO: 46)
LEDAEFRHDS GYEVHHQKLV FFAEDVGSNS GAIIGLMVGG
VVIATVIVIT LVML
(i) (C99APPA4-NOTCH1):
(SEQ ID NO: 100)
LEDAEFRHDS GYEVHHQKLV FFAEDVPPAQ GAIIGLMVGG
VVIATVIVIT LVML;
(j) (C99APPA4-SREBP1):
(SEQ ID NO: 101)
LEDAEFRHDS GYEVHHQKLV FFAEDVDRSR GAIIGLMVGG
VVIATVIVIT LVML;
(k) (C99GVP-APLP2-gsnk):
(SEQ ID NO: 19)
LEDAEFRHDS GLEEERESVG PLREDFGSNK GAIIGLMVGG
VVIATVIVIT LVML;
(l) (C99GVP-NOTCH1-gsnk):
(SEQ ID NO: 20)
LEDAEFRHDS GPYKIEAVQS ETVEPPGSNK GAIIGLMVGG
VVIATVIVIT LVML;
and
(m) (C99GVP-SREBP1-gsnk):
(SEQ ID NO: 21)
LEDAEFRHDS GAKPEQRPSL HSRGMLGSNK GAIIGLMVGG
VVIATVIVIT LVML.

12. The method of claim 4, wherein X2 is serine and X4 is lysine.

13. The method of claim 4, wherein X2 is leucine and X4 is serine.

14. The method of claim 4, wherein the first gamma secretase substrate is APP.

15. The method of claim 4 wherein JMD comprises the juxtamembrane domain of a gamma secretase substrate selected from APP, APLP2, Notch, erbB4, tyrosinase, p75 NTFR, SCNB2, n-cadherin, and CD44.

16. The method of claim 4 wherein [TMD] comprises the transmembrane domain of a gamma secretase substrate selected from APP, APLP2, Notch, erbB4, tyrosinase, p75 NTFR, SCNB2, n-cadherin, and CD44.

17. A method for determining whether a compound selectively inhibits gamma secretase activity of a first gamma secretase substrate relative to a second gamma secretase substrate, comprising:
(a) contacting a first gamma secretase substrate comprising a gamma secretase cleavage site with the compound at various concentrations and gamma secretase under conditions that allow for gamma secretase activity;
(b) separately contacting a second gamma secretase substrate comprising a gamma cleavage site with the compound at various concentrations and gamma secretase under conditions that allow for gamma secretase activity;
(c) measuring the intracellular domains (ICD) produced from each of the first and second gamma secretase substrates at each of the various compound concentrations to generate a first dose response curve of the effect of the compound on the first gamma secretase substrate and a second dose response curve of the effect of the compound on the second gamma secretase substrate; and
(d) comparing the first and second dose response curves,
wherein:
the first gamma secretase substrate comprises Formula II:
[JMDΔC4]-X1-X2-X3-X4-[TMD]
wherein
[JMDΔC4] comprises the amino acid sequence of a juxtamembrane domain (JMD) sequence of a gamma secretase substrate, wherein the JMD lacks the four C-terminal peptides;
[TMD] comprises a transmembrane domain sequence of a gamma secretase substrate; and
X1-X2-X3-X4 are independently selected from any amino acid; and
the second gamma secretase substrate comprises Formula II:
[JMDΔC4]-X1-X2-X3-X4-[TMD]
wherein X1-X2-X3-X4 are independently selected from any amino acid.

18. The method of claim 17, wherein
X1 is selected from S, T, G, P, Q, R, V, L, N, P, A, K, E, I, F, H, W, and D;
X2 is any amino acid;
X3 is selected from S, N, D, P, E, R, T, F, I, K, L, V, G, W, H, and A; and
X4 is any amino acid.

19. The method of claim 17, wherein
X1 is selected from S, T, G, P, Q, R, V, L, N, P, A, K, E, I, F, H, W, and D;
X3 is selected from S, N, D, P, E, R, T, F, I, K, L, V, G, W, H, and A; and
X2 and X4 are selected from L, I, H, E, V, A, S, T, D, N, P, K, Q, and R.

20. The method of claim 17, wherein
X1 is selected from S, T, G, P, Q, R, and D;
X2 is any amino acid;
X3 is selected from S, N, D, P, and A; and
X4 is any amino acid.

21. The method of claim 17, wherein X1-X2-X3-X4 of the first gamma secretase substrate is different from the X1-X2-X3-X4 of the second gamma secretase substrate.

22. The method of claim 17, wherein a shift in the second dose response curve toward a higher concentration relative to the first dose response curve indicates that the compound is selective for the first gamma secretase substrate relative to the second gamma secretase substrate.

23. The method of claim 17, wherein X1-X2-X3-X4 of the first and second gamma secretase substrate are independently selected from GLNK, SLSS, GSNK, GSNS, PPAQ, SSNK, GSSK, QHAR, QASR, TTDN, RDST, DVDR, or QIPE.

24. The method of claim 17, wherein [TMD] of the first and second gamma secretase substrate comprises SEQ ID NO:13.

25. The method of claim 17, wherein [JMDΔC4] of the first and second gamma secretase substrate are independently selected from SEQ ID NOs: 3-5, and 7-12.

26. The method of claim 17, wherein the gamma secretase substrate of Formula II comprises a sequence selected from the group consisting of:
(a) (C99GVP-APLP2):
(SEQ ID NO: 16)
LEDAEFRHDS GLEEERESVG PLREDFSLSS GAIIGLMVGG
VVIATVIVIT LVML;
(b) (C99GVP-NOTCH1):
(SEQ ID NO: 17)
LEDAEFRHDS GPYKIEAVQS ETVEPPPPAQ GAIIGLMVGG
VVIATVIVIT LVML;
(c) (C99GVP-SREBP1):
(SEQ ID NO: 18)
LEDAEFRHDS GAKPEQRPSL HSRGMLDRSR GAIIGLMVGG
VVIATVIVIT LVML;
(d) (C99APPA4-APLP2):
(SEQ ID NO: 42)
LEDAEFRHDS GYEVHHQKLV FFAEDVSLSS GAIIGLMVGG
VVIATVIVIT LVML;
(e) (C99-APP-(G25S):
(SEQ ID NO: 43)
LEDAEFRHDS GYEVHHQKLV FFAEDVSSNK GAIIGLMVGG
VVIATVIVIT LVML
(f) (C99-APP-(S26L):
(SEQ ID NO: 44)
LEDAEFRHDS GYEVHHQKLV FFAEDVGLNK GAIIGLMVGG
VVIATVIVIT LVML
(g) (C99-APP-(N27S):
(SEQ ID NO: 45)
LEDAEFRHDS GYEVHHQKLV FFAEDVGSSK GAIIGLMVGG
VVIATVIVIT LVML
(h) (C99-APP-(K285):
(SEQ ID NO: 46)
LEDAEFRHDS GYEVHHQKLV FFAEDVGSNS GAIIGLMVGG
VVIATVIVIT LVML
(i) (C99APPA4-NOTCH1):
(SEQ ID NO: 100)
LEDAEFRHDS GYEVHHQKLV FFAEDVPPAQ GAIIGLMVGG
VVIATVIVIT LVML;
(j) (C99APPA4-SREBP1):
(SEQ ID NO: 101)
LEDAEFRHDS GYEVHHQKLV FFAEDVDRSR GAIIGLMVGG
VVIATVIVIT LVML;
(k) (C99GVP-APLP2-gsnk):
(SEQ ID NO: 19)
LEDAEFRHDS GLEEERESVG PLREDFGSNK GAIIGLMVGG
VVIATVIVIT LVML;
(l) (C99GVP-NOTCH1-gsnk):
(SEQ ID NO: 20)
LEDAEFRHDS GPYKIEAVQS ETVEPPGSNK GAIIGLMVGG
VVIATVIVIT LVML;
and
(m) (C99GVP-SREBP1-gsnk):
(SEQ ID NO: 21)
LEDAEFRHDS GAKPEQRPSL HSRGMLGSNK GAIIGLMVGG
VVIATVIVIT LVML.

27. The method of claim 17, wherein X2 is serine and X4 is lysine.

28. The method of claim 17, wherein X2 is leucine and X4 is serine.

29. The method of claim 17 wherein JMD comprises the juxtamembrane domain of a gamma secretase substrate selected from APP, APLP2, Notch, erbB4, tyrosinase, p75 NTFR, SCNB2, n-cadherin, and CD44.

30. The method of claim 17 wherein [TMD] comprises the transmembrane domain of a gamma secretase substrate selected from APP, APLP2, Notch, erbB4, tyrosinase, p75 NTFR, SCNB2, n-cadherin, and CD44.


SEQUENCE LISTING (103 sequences)

SEQ ID NO: 1 - protein, 99 aa, Artificial
  Synthetic
DAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 2 - protein, 230 aa, Artificial
  Synthetic
KLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGIPGDLAPPTDVSLGDELHLDGEDVAMAHADALDDFDLDMLGDGDSPGPGFTPHDSAPYGALDMADFEFEQMFTDALGIDEYGG

SEQ ID NO: 3 - protein, 15 aa, Artificial
  Synthetic
YEVHHQKLVFFAEDV

SEQ ID NO: 4 - protein, 15 aa, Artificial
  Synthetic
LEEERESVGPLREDF

SEQ ID NO: 5 - protein, 15 aa, Artificial
  Synthetic
PYKIEAVQSETVEPP

SEQ ID NO: 6 - protein, 15 aa, Artificial
  Synthetic
AKPEQRPSLHSRGML

SEQ ID NO: 7 - protein, 15 aa, Artificial
  Synthetic
HDCIYYPWTGHSTLP

SEQ ID NO: 8 - protein, 15 aa, Artificial
  Synthetic
SDPDSFQDYIKSYLE

SEQ ID NO: 9 - protein, 14 aa, Artificial
  Synthetic
VTTVMGSSPVVTRG

SEQ ID NO: 10 - protein, 15 aa, Artificial
  Synthetic
HGKIHLQVLMEEPPE

SEQ ID NO: 11 - protein, 15 aa, Artificial
  Synthetic
LRVKVCQCDSNGDCT

SEQ ID NO: 12 - protein, 15 aa, Artificial
  Synthetic
QEGGANTTSGPIRTP

SEQ ID NO: 13 - protein, 24 aa, Artificial
  Synthetic
GAIIGLMVGGVVIATVIVITLVML

SEQ ID NO: 14 - protein, 9 aa, Artificial
  Synthetic
DAEFRHDSG

SEQ ID NO: 15 - protein, 54 aa, Artificial
  Synthetic
LEDAEFRHDSGYEVHHQKLVFFAEDVGSNKGAIIGLMVGGVVIATVIVITLVML

SEQ ID NO: 16 - protein, 54 aa, Artificial
  Synthetic
LEDAEFRHDSGLEEERESVGPLREDFSLSSGAIIGLMVGGVVIATVIVITLVML

SEQ ID NO: 17 - protein, 54 aa, Artificial
  Synthetic
LEDAEFRHDSGPYKIEAVQSETVEPPPPAQGAIIGLMVGGVVIATVIVITLVML

SEQ ID NO: 18 - protein, 54 aa, Artificial
  Synthetic
LEDAEFRHDSGAKPEQRPSLHSRGMLDRSRGAIIGLMVGGVVIATVIVITLVML

SEQ ID NO: 19 - protein, 54 aa, Artificial
  Synthetic
LEDAEFRHDSGLEEERESVGPLREDFGSNKGAIIGLMVGGVVIATVIVITLVML

SEQ ID NO: 20 - protein, 54 aa, Artificial
  Synthetic
LEDAEFRHDSGPYKIEAVQSETVEPPGSNKGAIIGLMVGGVVIATVIVITLVML

SEQ ID NO: 21 - protein, 54 aa, Artificial
  Synthetic
LEDAEFRHDSGAKPEQRPSLHSRGMLGSNKGAIIGLMVGGVVIATVIVITLVML

SEQ ID NO: 22 - protein, 19 aa, Artificial
  Synthetic
GYEVHHQKLVFFAEDGSNK

SEQ ID NO: 23 - protein, 19 aa, Artificial
  Synthetic
LEEERESVGPLREDFSLSS

SEQ ID NO: 24 - protein, 19 aa, Artificial
  Synthetic
PYKIEAVQSETVEPPPPAQ

SEQ ID NO: 25 - protein, 19 aa, Artificial
  Synthetic
AKPEQRPSLHSRGMLDRSR

SEQ ID NO: 26 - protein, 18 aa, Artificial
  Synthetic
VTTVMGSSPVVTRGTTDN

SEQ ID NO: 27 - protein, 19 aa, Artificial
  Synthetic
LRVKVCQCDSNGDCTDVDR

SEQ ID NO: 28 - protein, 19 aa, Artificial
  Synthetic
HGKIHLQVLMEEPPERDST

SEQ ID NO: 29 - protein, 19 aa, Artificial
  Synthetic
SDPDSFQDYIKSYLEQASR

SEQ ID NO: 30 - protein, 19 aa, Artificial
  Synthetic
QEGGANTTSGPIRTPQIPE

SEQ ID NO: 31 - protein, 19 aa, Artificial
  Synthetic
LEEERESVGPLREDFGSNK

SEQ ID NO: 32 - protein, 19 aa, Artificial
  Synthetic
PYKIEAVQSETVEPPGSNK

SEQ ID NO: 33 - protein, 19 aa, Artificial
  Synthetic
AKPEQRPSLHSRGMLGSNK

SEQ ID NO: 34 - protein, 19 aa, Artificial
  Synthetic
GYEVHHQKLVFFAEDSLSS

SEQ ID NO: 35 - protein, 19 aa, Artificial
  Synthetic
GYEVHHQKLVFFAEDDRSR

SEQ ID NO: 36 - protein, 19 aa, Artificial
  Synthetic
GYEVHHQKLVFFAEDPPAQ

SEQ ID NO: 37 - protein, 11 aa, Artificial
  Synthetic
LEDAEFRHDSG

SEQ ID NO: 38 - protein, 88 aa, Artificial
  Synthetic
VHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 39 - protein, 7 aa, Artificial
  Synthetic
VMLKKKC

SEQ ID NO: 40 - protein, 13 aa, Artificial
  Synthetic
GYENPTYKFFEQM

SEQ ID NO: 41 - protein, 50 aa, Artificial
  Synthetic
VMLKKKQYTSIHHGVVEVDAAVTPEERHLSKMQQNGYENPTYKFFEQMQN

SEQ ID NO: 42 - protein, 54 aa, Artificial
  Synthetic
LEDAEFRHDSGYEVHHQKLVFFAEDVSLSSGAIIGLMVGGVVIATVIVITLVML

SEQ ID NO: 43 - protein, 54 aa, Artificial
  Synthetic
LEDAEFRHDSGYEVHHQKLVFFAEDVSSNKGAIIGLMVGGVVIATVIVITLVML

SEQ ID NO: 44 - protein, 54 aa, Artificial
  Synthetic
LEDAEFRHDSGYEVHHQKLVFFAEDVGLNKGAIIGLMVGGVVIATVIVITLVML

SEQ ID NO: 45 - protein, 54 aa, Artificial
  Synthetic
LEDAEFRHDSGYEVHHQKLVFFAEDVGSSKGAIIGLMVGGVVIATVIVITLVML

SEQ ID NO: 46 - protein, 54 aa, Artificial
  Synthetic
LEDAEFRHDSGYEVHHQKLVFFAEDVGSNSGAIIGLMVGGVVIATVIVITLVML

SEQ ID NO: 47 - protein, 329 aa, Artificial
  Synthetic
DAEFRHDSGYEVHHQKLVFFAEDVGSNKGAIIGLMVGGVVIATVIVITLVMLKKKKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGIPGDLAPPTDVSLGDELHLDGEDVAMAHADALDDFDLDMLGDGDSPGPGFTPHDSAPYGALDMADFEFEQMFTDALGIDEYGGQYTSIHHGVVEVDAAVTPEERHLSKMQQNGYENPTYKFFEQMQN

SEQ ID NO: 48 - protein, 329 aa, Artificial
  Synthetic
DAEFRHDSGLEEERESVGPLREDFSLSSGAIIGLMVGGVVIATVIVITLVMLKKKKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGIPGDLAPPTDVSLGDELHLDGEDVAMAHADALDDFDLDMLGDGDSPGPGFTPHDSAPYGALDMADFEFEQMFTDALGIDEYGGQYTSIHHGVVEVDAAVTPEERHLSKMQQNGYENPTYKFFEQMQN

SEQ ID NO: 49 - protein, 329 aa, Artificial
  Synthetic
DAEFRHDSGPYKIEAVQSETVEPPPPAQGAIIGLMVGGVVIATVIVITLVMLKKKKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGIPGDLAPPTDVSLGDELHLDGEDVAMAHADALDDFDLDMLGDGDSPGPGFTPHDSAPYGALDMADFEFEQMFTDALGIDEYGGQYTSIHHGVVEVDAAVTPEERHLSKMQQNGYENPTYKFFEQMQN

SEQ ID NO: 50 - protein, 329 aa, Artificial
  Synthetic
DAEFRHDSGPYKIEAVQSETVEPPGSNKGAIIGLMVGGVVIATVIVITLVMLKKKKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGIPGDLAPPTDVSLGDELHLDGEDVAMAHADALDDFDLDMLGDGDSPGPGFTPHDSAPYGALDMADFEFEQMFTDALGIDEYGGQYTSIHHGVVEVDAAVTPEERHLSKMQQNGYENPTYKFFEQMQN

SEQ ID NO: 51 - protein, 329 aa, Artificial
  Synthetic
DAEFRHDSGYEVHHQKLVFFAEDVSLSSGAIIGLMVGGVVIATVIVITLVMLKKKKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGIPGDLAPPTDVSLGDELHLDGEDVAMAHADALDDFDLDMLGDGDSPGPGFTPHDSAPYGALDMADFEFEQMFTDALGIDEYGGQYTSIHHGVVEVDAAVTPEERHLSKMQQNGYENPTYKFFEQMQN

SEQ ID NO: 52 - DNA, 60 bp, Artificial
  Synthetic
cggctcgggcgctggaggatgcagaattccgacatgactcaggatatgaagttcatcatc

SEQ ID NO: 53 - DNA, 51 bp, Artificial
  Synthetic
tggcactgctcctgctggccgcctggacggctcgggcgctggaggatgcag

SEQ ID NO: 54 - DNA, 48 bp, Artificial
  Synthetic
caccaccatgctgcccggtttggcactgctcctgctggccgcctggac

SEQ ID NO: 55 - DNA, 33 bp, Artificial
  Synthetic
gcggccgcctagttctgcatctgctcaaagaac

SEQ ID NO: 56 - DNA, 60 bp, Artificial
  Synthetic
ccgtgggcccactgcgggaggacttcagtctgagtagcggtgcaatcattggactcatgg

SEQ ID NO: 57 - DNA, 59 bp, Artificial
  Synthetic
tcccgcagtgggcccacggattcccgctcttcctcgagtcctgagtcatgtcggaattc

SEQ ID NO: 58 - DNA, 60 bp, Artificial
  Synthetic
tgcagagtgagaccgtggagccgcccccgccggcgcagggtgcaatcattggactcatgg

SEQ ID NO: 59 - DNA, 59 bp, Artificial
  Synthetic
tccacggtctcactctgcacggcctcgatcttgtagggtcctgagtcatgtcggaattc

SEQ ID NO: 60 - DNA, 60 bp, Artificial
  Synthetic
cgtctctgcacagccggggcatgctggaccgctcccgcggtgcaatcattggactcatgg

SEQ ID NO: 61 - DNA, 59 bp, Artificial
  Synthetic
ccccggctgtgcagagacggccgctgctctggctttgctcctgagtcatgtcggaattc

SEQ ID NO: 62 - DNA, 63 bp, Artificial
  Synthetic
cgggtcaccacgggctgggagctgcccatcactgtggtcactcctgagtcatgtcggaattct

SEQ ID NO: 63 - DNA, 64 bp, Artificial
  Synthetic
gcagctcccagcccgtggtgacccgaggcaccaccgacaacggtgcaatcattggactcatggt

SEQ ID NO: 64 - DNA, 65 bp, Artificial
  Synthetic
cagtccccgttggagtcacactggcaaaccttcacgcgcagtcctgagtcatgtcggaattctgc

SEQ ID NO: 65 - DNA, 66 bp, Artificial
  Synthetic
ttgccagtgtgactccaacggggactgcacagatgtggacaggggtgcaatcattggactcatggt

SEQ ID NO: 66 - DNA, 66 bp, Artificial
  Synthetic
taaagtggaatggcccgtccatgggtagtaaatgcagtcatgtcctgagtcatgtcggaattctgc

SEQ ID NO: 67 - DNA, 65 bp, Artificial
  Synthetic
tacccatggacgggccattccactttaccacaacatgctagaggtgcaatcattggactcatggt

SEQ ID NO: 68 - DNA, 70 bp, Artificial
  Synthetic
gttccaaataggacttaatgtagtcttgaaaagagtctgggtctgatcctgagtcatgtcggaattctgc

SEQ ID NO: 69 - DNA, 67 bp, Artificial
  Synthetic
cttttcaagactacattaagtcctatttggaacaagcgagtcggggtgcaatcattggactcatggt

SEQ ID NO: 70 - DNA, 66 bp, Artificial
  Synthetic
agggggctcttccatgaggacctgcagatggatcttgccatgtcctgagtcatgtcggaattctgc

SEQ ID NO: 71 - DNA, 65 bp, Artificial
  Synthetic
ctgcaggtcctcatggaagagccccctgagcgggactccacgggtgcaatcattggactcatggt

SEQ ID NO: 72 - DNA, 55 bp, Artificial
  Synthetic
cgggcgctggaggatgcagaattcgcagctgcctcaggatatgaagttcatcatc

SEQ ID NO: 73 - DNA, 55 bp, Artificial
  Synthetic
gatgatgaacttcatatcctgaggcagctgcgaattctgcatcctccagcgcccg

SEQ ID NO: 74 - DNA, 44 bp, Artificial
  Synthetic
gttgctgctcaagcattggtgttctttgcagaagatgtgggttc

SEQ ID NO: 75 - DNA, 58 bp, Artificial
  Synthetic
gaacaccaatgcttgagcagcaacttcatatcctgagtcatgtcggaattctgcatcc

SEQ ID NO: 76 - DNA, 39 bp, Artificial
  Synthetic
ggtgttctttgcagcagctgtgggttcaaacaaaggtgc

SEQ ID NO: 77 - DNA, 39 bp, Artificial
  Synthetic
gcacctttgtttgaacccacagctgctgcaaagaacacc

SEQ ID NO: 78 - DNA, 59 bp, Artificial
  Synthetic
ccgtgggcccactgcgggaggacttcggttcaaacaaaggtgcaatcattggactcatg

SEQ ID NO: 79 - DNA, 60 bp, Artificial
  Synthetic
tgcagagtgagaccgtggagccgcccggttcaaacaaaggtgcaatcattggactcatgg

SEQ ID NO: 80 - DNA, 60 bp, Artificial
  Synthetic
cgtctctgcacagccggggcatgctgggttcaaacaaaggtgcaatcattggactcatgg

SEQ ID NO: 81 - DNA, 49 bp, Artificial
  Synthetic
agaagatgtgagtctgagtagcggtgcaatcattggactcatggtgggc

SEQ ID NO: 82 - DNA, 57 bp, Artificial
  Synthetic
tgcaccgctactcagactcacatcttctgcaaagaacaccaatttttgatgatgaac

SEQ ID NO: 83 - DNA, 48 bp, Artificial
  Synthetic
ggtgttctttgcagaagatgtgagttcaaacaaaggtgcaatcattgg

SEQ ID NO: 84 - DNA, 48 bp, Artificial
  Synthetic
ccaatgattgcacctttgtttgaactcacatcttctgcaaagaacacc

SEQ ID NO: 85 - DNA, 50 bp, Artificial
  Synthetic
ggtgttctttgcagaagatgtgggtttaaacaaaggtgcaatcattggac

SEQ ID NO: 86 - DNA, 50 bp, Artificial
  Synthetic
gtccaatgattgcacctttgtttaaacccacatcttctgcaaagaacacc

SEQ ID NO: 87 - DNA, 52 bp, Artificial
  Synthetic
ggtgttctttgcagaagatgtgggttcaagcaaaggtgcaatcattggactc

SEQ ID NO: 88 - DNA, 52 bp, Artificial
  Synthetic
gagtccaatgattgcacctttgcttgaacccacatcttctgcaaagaacacc

SEQ ID NO: 89 - DNA, 56 bp, Artificial
  Synthetic
ggtgttctttgcagaagatgtgggttcaaactcaggtgcaatcattggactcatgg

SEQ ID NO: 90 - DNA, 56 bp, Artificial
  Synthetic
ccatgagtccaatgattgcacctgagtttgaacccacatcttctgcaaagaacacc

SEQ ID NO: 91 - protein, 18 aa, Artificial
  Synthetic
TVIVITLVMLKKKQTYTS

SEQ ID NO: 92 - protein, 125 aa, Artificial
  Synthetic
ADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 93 - protein, 14 aa, Artificial
  Synthetic
CGYENPTYKFFEQM

SEQ ID NO: 94 - protein, 4 aa, Artificial
  Synthetic
QHAR

SEQ ID NO: 95 - protein, 4 aa, Artificial
  Synthetic
QASR

SEQ ID NO: 96 - protein, 4 aa, Artificial
  Synthetic
TTDN

SEQ ID NO: 97 - protein, 4 aa, Artificial
  Synthetic
RDST

SEQ ID NO: 98 - protein, 4 aa, Artificial
  Synthetic
DVDR

SEQ ID NO: 99 - protein, 4 aa, Artificial
  Synthetic
QIPE

SEQ ID NO: 100 - protein, 4 aa, Artificial
  Synthetic
PPAQ

SEQ ID NO: 101 - protein, 4 aa, Artificial
  Synthetic
DRSR

SEQ ID NO: 102 - protein, 4 aa, Artificial
  Synthetic
SLSS

SEQ ID NO: 103 - protein, 4 aa, Artificial
  Synthetic
GSNK